SEQUENCE LISTING (36 sequences)

SEQ ID NO: 1 - DNA, 3450 bp, Crotalaria juncea
  source 1..3450: mol_type = genomic DNA
atggcatcttcatcaactcaaataaaatatgatgtgttcattagctttaggggcacagacataaggtatggttttcttagccatttgaaaaaggaactaaggcagaaacaggttgatgcctttgtggatgataggcttgagagtggggatgaaatatcactagcacttgtcaaagccattgaagaatcacttatttctttggtcatattctcaaaagactatgcttcttcaaagtggtgcttggaagagctagtgaaaataattgaatgcatggctagaaataaacaacttgttattcctattttctacaatgttgatccatctgatgtacgccaccaaaaagggacctatggagatgcgtttgccaaacatgataaaaataaaaggaatctggcaaaggtgccaaattggagatatgctttgaatatagcggctaatctttctggatttcattcatcaaattatggggatgaagttgagcttatagaagatattgtcaaacgtttgtcaacaaggttgaatcttatgtaccaaagtgagttaacataccttgttggaattgaagaacggattgcagatattgagtcactattgttcccggatcatgcaataatttctcccacgacagatgtacgtgttattgggatttggggcatggggggtattggcaagacaactattgcagctgcagtgttcaaccgtttgtgttttgaatatgaagggtattgctttatggcaaacataagagaagaatcagagaaacatggaattattcatttgaagaataaaattctatctatactattaaaggaaaatgatttatatattggtacacctaatggagttcccccttatgttaagagaaggctgctaagaaaaaaagttcttgttgttcttgatgatgttaatgactctgaacaactagagaatttggttggagaacttgattggttcggatccggtagcagaatcattgtgacaactagagataagcaagtgcttggtaagagagttgatgctatatatggtgccaaggccttaaattttgatgaagctgttaggcttttcattttgaatgctttcaaagaaagaggttgtcttgacatggagtggattgagctatcaagaagggtgattcagtatgctaatggaaatccattagccttgaaagttttgggttcttttctttatggtaaaagcaaaatagagtgggaaagccaattgcagaagcttaagaaaatgcctcatgcaaaaattcagaatgttttgagattgagttatgataagttggatagagaagagaagaatatatttctatatgttgcttgttttttgaaaggatatgaatcacgccgaataatagttttgctcgatgcttgtggtttctcaacaataattgggttaaaagtccttcaagacaaagcccttatagtggaagcaaaggggtcaggaagatcgatcgtatcgatgcatgacttgatacaagagatgggttgggagattgttagagaagaatctattgaagatcctgggaaacgcagcaggttatgggatcctagtgatattcatcatgtattgaaaaataacaagggaactaaagccattaaaagcataactctgaatgtgtcaaaatttgatgagctatgcttaagtcctcaagtctttgctaggatgcaacagttaaaatttcttaatttttcccaacattacggcgatgagcaaatcctgtatcttcctcaaggccttgaatctttgccaaatgacctaagactttttcgttgggttagttatcctttgaaatccttgcctgaaacattctgtgcagaaaatcttgttgagctcaacatgacttggagcagagctgaaaaactttgggatggagtccagaatcttcaacatttaaagaaaattgacctcagtaattccaaaaatttgctagagctgccagatttttccaaggccacaaatcttgaagaagtagaactatttggttgcagaaatttgcttaatgttcatccatctattttatctctccacaagctcgtgagattaaacttattcttctgcaaagcactcaccagtcttcgaagtgatactcatttaagatcccttcgtgacctcttcctcggtggttgctcgaggctcaaggagttttcgttgacctcagagaacatgaaagacttgaacttaaactcaactgctatcaacgaattgccatcatcaatcggaagactccgaaaacttgaaactctgactcttgatcattgcaaaagcctaaacaatcttccaaacaaggttgctgaccttacatcacttcgcgttcttcgtatatatggttgcatacaactcgatgcgtcaaatcttcatgtcctatttaatggtttaaggtctttggaaacgttactggtagaagaatgtctcaacttaaatgaaattcctgaaaacataagcctcctatgttcattgcatcatttattactgaaaggaacaaatgttgagagattccctgcaagtatcaaacaacttacaaagctagaaaagcttgacataagtggttgcaagaggcttcattctctaccagaacttccactatccataaaagagttatatgccgctaactgcacatcgttggagactgtaatgttccctttgagggctgctgaactagtacaagtacatgcatgtaagacatacactaaattcgaaaattgcataaagttggatcgacattcactcaatgctattggattaaatgctcatgtgggcatgaagaaattggtatatgaacatctctcaaatataggaagcaagttccttgatggtccagtttatgttatttacccgggaagcgaagttccggagtggttcatggacagaacaacacaggcttcaataaccattgatctctcttctcctgcaccacattccaaagttatgggcttcatcttttgtgtcattgctggtcagtttccatcaaatgacaagaacttcattggatgtgactgctacctagaaactggtactaatggtgaaagggtcagccttggacacatgaataataatgcatggtctagtatacatgcaggtgaatttgtttctgatcatgtgtgcatgtggtttgatgaacaatgttgtcttcagaaccgcaaatgcgaaaatgaaagcatggaagaagaagagcttgtggctagctataatccaaaggtttgttttgaattctttgctcaaagtggaagtacttgggaaaagggggaggatattataataaaagggtgtggagtgtgtccaatttatgactcagaatatcatgatttcatcaaacaattggagttggagttgaaagaaacacaaaccctgaagcagcaatgcaaggaactcatgtttccatatctacaagttggaaactggaagagtgcaacacagggtttgaaagatatattgcttctatga

SEQ ID NO: 2 - protein, 1149 aa, Crotalaria juncea
MASSSTQIKYDVFISFRGTDIRYGFLSHLKKELRQKQVDAFVDDRLESGDEISLALVKAIEESLISLVIFSKDYASSKWCLEELVKIIECMARNKQLVIPIFYNVDPSDVRHQKGTYGDAFAKHDKNKRNLAKVPNWRYALNIAANLSGFHSSNYGDEVELIEDIVKRLSTRLNLMYQSELTYLVGIEERIADIESLLFPDHAIISPTTDVRVIGIWGMGGIGKTTIAAAVFNRLCFEYEGYCFMANIREESEKHGIIHLKNKILSILLKENDLYIGTPNGVPPYVKRRLLRKKVLVVLDDVNDSEQLENLVGELDWFGSGSRIIVTTRDKQVLGKRVDAIYGAKALNFDEAVRLFILNAFKERGCLDMEWIELSRRVIQYANGNPLALKVLGSFLYGKSKIEWESQLQKLKKMPHAKIQNVLRLSYDKLDREEKNIFLYVACFLKGYESRRIIVLLDACGFSTIIGLKVLQDKALIVEAKGSGRSIVSMHDLIQEMGWEIVREESIEDPGKRSRLWDPSDIHHVLKNNKGTKAIKSITLNVSKFDELCLSPQVFARMQQLKFLNFSQHYGDEQILYLPQGLESLPNDLRLFRWVSYPLKSLPETFCAENLVELNMTWSRAEKLWDGVQNLQHLKKIDLSNSKNLLELPDFSKATNLEEVELFGCRNLLNVHPSILSLHKLVRLNLFFCKALTSLRSDTHLRSLRDLFLGGCSRLKEFSLTSENMKDLNLNSTAINELPSSIGRLRKLETLTLDHCKSLNNLPNKVADLTSLRVLRIYGCIQLDASNLHVLFNGLRSLETLLVEECLNLNEIPENISLLCSLHHLLLKGTNVERFPASIKQLTKLEKLDISGCKRLHSLPELPLSIKELYAANCTSLETVMFPLRAAELVQVHACKTYTKFENCIKLDRHSLNAIGLNAHVGMKKLVYEHLSNIGSKFLDGPVYVIYPGSEVPEWFMDRTTQASITIDLSSPAPHSKVMGFIFCVIAGQFPSNDKNFIGCDCYLETGTNGERVSLGHMNNNAWSSIHAGEFVSDHVCMWFDEQCCLQNRKCENESMEEEELVASYNPKVCFEFFAQSGSTWEKGEDIIIKGCGVCPIYDSEYHDFIKQLELELKETQTLKQQCKELMFPYLQVGNWKSATQGLKDILLL

SEQ ID NO: 3 - DNA, 3426 bp, Lupinus angustifolius
  source 1..3426: mol_type = genomic DNA
atggaatcttcatcttcctgtggtgtaactcaaatcaaatatgatgtcttcattagctttagaggcacagatatacgctacggttttctcagccatttgaaaaaggaactaaggcagaagcaagttgatgctttcgtagatgatagacttgagagtggtgatgaaatatcaccagcacttgtaaaagctattgaacaatcacttatttctttgatcatattctcaaaagactatgcttcttcaaagtggtgcttggaagagcttgtgaaaatagttgagtgcatggcaaggaataaacaaattgttgttccagttttctataatgttgatccatctcatgtacgctatcagaaagggacttatggtgatgcatttgctatacatgagaaaaagaaaaggaacttgtctaagttgcaaaattggagatctgctttgaatatagctgctaatctttctggatttcattcatcaaattttggggatgaagttgagcttatagaagaagttgtgaaatgtttgttggcaaggttgaatcttatgtaccaaagtgagttaacagacctcgttggaatcgaagaacggattgcagatatagagtcactattgttcctgaaatcgacaatagatgttcgtgtgattgggatttggggcatggggggtattggtaaaacaactattgcagctgcagtatttaaccgtttgtgtttcgagtatgaagggtgttgctttatggcaaacataagagaagaatcagagaaacatggaactatttctttgaagaataaaattctgtctatactattaaaggaaaatgatctatatatcggtacgcctaatggagttcctccttatgtaaagagacggctgcttcgaaaaaaggttcttgttgttcttgatgatattaatgactctgagcaactagagaatttggttggagaactcgattggttcggacccggtagcagaatcattgttacaactagagataaacaagtgcttggtaaaagagttgatagcatctatgaggctaaggcattaaattttgatgaagctgttaagcttttcattatgaatgccttcaaacaaattagttctcttgacatggagtggattgagctatcaagaagggtgattcagtatgctaatggaaatccattagccttaaaggttttgggctcttttctttacggtaaaacgaaaatagagtgggaaagtcaattgcaaaaacttaagaaaatgcctcatgcaaaaattcagaatgttttgagattgacttatgataagttggatagagaagagaagaacatatttctatatgttgcatgttttctgaaaggatatgaatcacatcgaataatagttctgcttgacgcttgtggtttctcgacaattattgggctaaaagtccttcgagacaaagcccttgtagttgaagccaagggatcaggaagatcaattgtatcaatgcatgatttgatacaagaaatgggttgggagattgttagagaagaatctatcgatgaccccggaaaacgcagccggctatgggatcctactgatattcatcaagtattgaaaaataactcaggaactaaagccattaaaagcataacactgaatgtgtcaaaaattggtgaactttgcttaagtcctcaagttttttctaggatgcaacaattgaaatttcttaatttttcgcaaccttatggcgatgagcaaatcttgtatcttcctcaaggccttgaatctttgccaaatgacctaagactttttcattggattagttatcctttgaaatccttgcctattacattctgtgcagaaaatcttgttgagctcaacatgacttggagcagagccgaaaaactttgggatggaatccagaatcttcagcatttaaagaaaattgacctcagtaattcgaaacatttgctagagctaccggatttttcaaaggccacaaatcttgaagaaatagaactatttggttgcagaaatttacttaatgttcatccatctattttatcacttcacaagcttgtaagattaaacctgtttttttgcaaagcactcactagtcttcgaagcgatactcatttaagactccgtgatctcttcctcggtggttgttcaaggctcaaggagttttcactgacttcagagaacatgaaagacttgaacttaaattcaactgctatcaatgaattgccatcatcaattgggagactaaaaaaacttgaaactttggcacttgatcactgcaaaggcctaaacaatcttccagacaaggttgccgaccttggatcgctacgtgttctccgtatatatggttgcatacaacttgacgcatcaaatctgcatgtcttatttaatggtttaaggtctttggaaacactactgctagaagaatgtcttaacttacatgaaattccagacaacataagtctcctatgttcattgcatcatttgttactcaaaggaacaaatgttgagagattccctacaagcattaagcatcttacgaaactagaaaaactcgacttaagtggttgcaagaggctgcattttctaccagaacttccactatccataaaagagttatatgccactaattgctcatctttggagactgtaatgttccctttgagaactactgatctagtacatgcatataagtcatacactacattccaaaattgcgtaaagctagataaacattcactcaaagctatcggagtaaacgcgcttgtcgacataaagaaattggcatatgaacatctctcaaatattggaaccaagtaccttgatggtccagcttatgttatctacccaggaaatcaagttccagagtcgttccgggacaggacaacacaggcttcagtaaccattgatctctcttctgctccttcatgttccaaggttataggtttcatcttctgtgtcattgttggtaagtttccatcaaatgacaagaacttcattggatgtgactgctacctagaaacaggtaatggtgaaagggttagccttggacacaagatgaacaatgcatggtctagtatacatgcttgtgaatttctttctgatcatgtgtgcatgtggttcgacgaacaatgttgtctgcaaaactgcgagcgccaagacgaaaacatcgaagaacttgtggcaaaggttacatttgagtattttgctcaatctggtagtagttgggaaaaagagaatgatattatgataaaagggtgtggagtgtgtccaatatatgactcagaatatcatgatttcatcaaacaaatggagctagacttgacactgcagaatggaacacaatatctgaaacagaaatgcaaggcattcatatttccacctctacaaagtgaaaactggaagagtgcaacacaaggcttgaaggaaatattgtttctatga

SEQ ID NO: 4 - protein, 1141 aa, Lupinus angustifolius
MESSSSCGVTQIKYDVFISFRGTDIRYGFLSHLKKELRQKQVDAFVDDRLESGDEISPALVKAIEQSLISLIIFSKDYASSKWCLEELVKIVECMARNKQIVVPVFYNVDPSHVRYQKGTYGDAFAIHEKKKRNLSKLQNWRSALNIAANLSGFHSSNFGDEVELIEEVVKCLLARLNLMYQSELTDLVGIEERIADIESLLFLKSTIDVRVIGIWGMGGIGKTTIAAAVFNRLCFEYEGCCFMANIREESEKHGTISLKNKILSILLKENDLYIGTPNGVPPYVKRRLLRKKVLVVLDDINDSEQLENLVGELDWFGPGSRIIVTTRDKQVLGKRVDSIYEAKALNFDEAVKLFIMNAFKQISSLDMEWIELSRRVIQYANGNPLALKVLGSFLYGKTKIEWESQLQKLKKMPHAKIQNVLRLTYDKLDREEKNIFLYVACFLKGYESHRIIVLLDACGFSTIIGLKVLRDKALVVEAKGSGRSIVSMHDLIQEMGWEIVREESIDDPGKRSRLWDPTDIHQVLKNNSGTKAIKSITLNVSKIGELCLSPQVFSRMQQLKFLNFSQPYGDEQILYLPQGLESLPNDLRLFHWISYPLKSLPITFCAENLVELNMTWSRAEKLWDGIQNLQHLKKIDLSNSKHLLELPDFSKATNLEEIELFGCRNLLNVHPSILSLHKLVRLNLFFCKALTSLRSDTHLRLRDLFLGGCSRLKEFSLTSENMKDLNLNSTAINELPSSIGRLKKLETLALDHCKGLNNLPDKVADLGSLRVLRIYGCIQLDASNLHVLFNGLRSLETLLLEECLNLHEIPDNISLLCSLHHLLLKGTNVERFPTSIKHLTKLEKLDLSGCKRLHFLPELPLSIKELYATNCSSLETVMFPLRTTDLVHAYKSYTTFQNCVKLDKHSLKAIGVNALVDIKKLAYEHLSNIGTKYLDGPAYVIYPGNQVPESFRDRTTQASVTIDLSSAPSCSKVIGFIFCVIVGKFPSNDKNFIGCDCYLETGNGERVSLGHKMNNAWSSIHACEFLSDHVCMWFDEQCCLQNCERQDENIEELVAKVTFEYFAQSGSSWEKENDIMIKGCGVCPIYDSEYHDFIKQMELDLTLQNGTQYLKQKCKAFIFPPLQSENWKSATQGLKEILFL

SEQ ID NO: 5 - DNA, 3450 bp, Lupinus albus
  source 1..3450: mol_type = genomic DNA
atggcatcttcatcttcatcttcctatggtgttactcaaatcaaatatgatgtctttattagctttaggggcacagatatacgatatggttttcttagccatttgaaaaaggaactaaagcagaaacaggttgatgcttttgtagatgataggcttgagagtggggatgaaatatcacctgcacttgtaaaagccattgaacaatcacttatttctttgatcattttctcaaaagactatgcttcttcaaagtggtgcttggaagagcttgtcaaaatagttgagtgcatggcaaggaataaacaaattgttcttccagttttctataatgttgatccatctgatgtacgctatcagaaagggacttatggagatgcatttgctatacatgagaaaaataagaggaagttgtccaagttgcaaaattggagatctgctttgaatatagctgctaatctttctggatttcattcatcaaaatttggggatgaagttgagcttatagaagaagttgtgaaatgtttgttggcaaggttgaatcttatgtaccaaagtgagttaacagaccttgttggaattgaagaacggattgcagatatagagtcactattgttcctgacatcgacaatagatgtacgtgtgattgggatttggggcatggggggtattggtaaaacaacaattgcagctgcagtatttaaccgtttgtgttttgagtatgaagggtgttgctttatggcaaacataagagaagaatcagagaaacatggcaccatttcattgaagaataaaattctctcaatactattaaaggaaaatgatctatatatcagtacgcctaatggagttcctccttatgtaaagagaaggctgcttcgcataaaggttcttgttgttcttgatgatattaatgactctgagcagctagagaatttagttggtgaacttgattggttcggacccggtagtagaatcattgtgacaactagagataaacaagtgcttggtaaaagagttgatagtatctatgaggctaaggcattaaattttgatgaagctgttaagcttttcattttgaatgccttcaaacaaattagttgtcttgacatggaatggattgagctatcaagaaaagtgattcagtatgctaatggaaatccattagctttaaaagttttgggctcttttctttacggtaaaacgaaaatagagtgggaaagtcaattgcaaaaacttaagaaaatgcctcatgcaaaaattcagaatgttttgagattgacttttgataagttggatagggaagagaagaacatatttctatatgttgcatgttttctgaagggatatgaatcacaccgaataatagttctgcttgatgcttgtggtttctcaacaatcattgggctaagagtccttcaagataaagcccttatagttgaagccaagggatcaggaagatcaattgtatcaatgcatgatttgatacaagaaatgggctgggagatagttagagaagaatctattgacgaccctggaaaacgcagcaggctatgggatcctagtgatattcatcaagtattgaaaaataacacaggaactaaagccattaaaagcataactctgaatgtgtcaaaaattggtgaactgtgcttaagtcctcaagtctttgctaggatgccacagttgaaatttcttaatttttcccaaccttatggcgatgagcaaattttgtatcttcctcaaggccttgaatctttgccaaatgacctaagactttttcattggattagttatcctttgaaatccttgcctattactttctgtgcagaaaatcttgttgagctcaacatgacttggagcagagcggaaaaactttgggatggaatccagaatcttcaacatttaaagaaaattgacctcagtaattccaaacatttgctagagctaccagatttttcaaaggccacaaatcttgaagaaatagaactatctggttgcagaaatttgcttaatgttcatccatctatattatccctccacaagcttgtaagattaaacctgtttttttgcaaagcactcaccagtattcgaagcgatactcatttaagatccctccgtgatctcttcctcggtggttgctcaaggctcaaggagttttcactgacttcagaaaatatgaaagacttgaacttaaattcaactgctatcaatgaattgccatcatcaattgggagactaaaaaaacttgaaactttgacacttgatcactgcaaaggcctaaacaatcttccagacaaggttgctgaccttggatcgctacgtgttcttcgtatatatggttgcatacaacttgacgcatcaaatcttcatgtcttatttaatggtttaaggtctttggaaacactactgctagaagaatgtcttaacttacatgaaattccagacaacataagtctcctatgttcattgcatcatttgttactgaaaggaacaaatgttgagagattccctacaagcattaagcatcttacaaagctagaaaagcttgacttaagtggttgcaagaggcttaattttctaccagaacttccactatccataaaagagttatatgccactaactgctcatctttggagactgtaatgttccctttgaggactactgatctagtacatgcatataagtcatacactacattccaaaattgcatgaacttggataaacattcactcaatgctattggagtagatgcacttgtcaacataaagaaattggcatatgaacatctctcaaatattggaagcaagttccttgatggtccagcttatgttatctacccaggaaatgaagttccagattggttcagggacaagacaacacaagctgcagtaactattgatctctcttctgctccttcatgttccaaggttatgggtttcatcttctgtgtcattgttggtcagtttccatcaaatgacaagaacttcattggatgtgactgctacctagaaacaggtaatggtgaaagggttagccttggacacaagatgaataatgcatggtctagtatacatgcttgtgaatttctttctgatcatgtgtgcatgtggttcgacgaacaatgttgtctgcaaaaccgcgagtgccaagacgaaagcatggaagaacttgttccaaaggttacatttgatttttttgctcaatccggtagtacatgggaaaaagagaatgatattatgataaaagggtgtggagtgtgtccaatatatgactcagaatataatgatttcattaaacaaatggagctggagttgacactgcaatgcagctcatatcagaatgaaacacaatctctgaaacagaaatgcaaggcattcatctttccacctctacaaattgaaaactggaagagtgcaacacaaggcttgaaggaaatattgtttctatga

SEQ ID NO: 6 - protein, 1149 aa, Lupinus albus
MASSSSSSYGVTQIKYDVFISFRGTDIRYGFLSHLKKELKQKQVDAFVDDRLESGDEISPALVKAIEQSLISLIIFSKDYASSKWCLEELVKIVECMARNKQIVLPVFYNVDPSDVRYQKGTYGDAFAIHEKNKRKLSKLQNWRSALNIAANLSGFHSSKFGDEVELIEEVVKCLLARLNLMYQSELTDLVGIEERIADIESLLFLTSTIDVRVIGIWGMGGIGKTTIAAAVFNRLCFEYEGCCFMANIREESEKHGTISLKNKILSILLKENDLYISTPNGVPPYVKRRLLRIKVLVVLDDINDSEQLENLVGELDWFGPGSRIIVTTRDKQVLGKRVDSIYEAKALNFDEAVKLFILNAFKQISCLDMEWIELSRKVIQYANGNPLALKVLGSFLYGKTKIEWESQLQKLKKMPHAKIQNVLRLTFDKLDREEKNIFLYVACFLKGYESHRIIVLLDACGFSTIIGLRVLQDKALIVEAKGSGRSIVSMHDLIQEMGWEIVREESIDDPGKRSRLWDPSDIHQVLKNNTGTKAIKSITLNVSKIGELCLSPQVFARMPQLKFLNFSQPYGDEQILYLPQGLESLPNDLRLFHWISYPLKSLPITFCAENLVELNMTWSRAEKLWDGIQNLQHLKKIDLSNSKHLLELPDFSKATNLEEIELSGCRNLLNVHPSILSLHKLVRLNLFFCKALTSIRSDTHLRSLRDLFLGGCSRLKEFSLTSENMKDLNLNSTAINELPSSIGRLKKLETLTLDHCKGLNNLPDKVADLGSLRVLRIYGCIQLDASNLHVLFNGLRSLETLLLEECLNLHEIPDNISLLCSLHHLLLKGTNVERFPTSIKHLTKLEKLDLSGCKRLNFLPELPLSIKELYATNCSSLETVMFPLRTTDLVHAYKSYTTFQNCMNLDKHSLNAIGVDALVNIKKLAYEHLSNIGSKFLDGPAYVIYPGNEVPDWFRDKTTQAAVTIDLSSAPSCSKVMGFIFCVIVGQFPSNDKNFIGCDCYLETGNGERVSLGHKMNNAWSSIHACEFLSDHVCMWFDEQCCLQNRECQDESMEELVPKVTFDFFAQSGSTWEKENDIMIKGCGVCPIYDSEYNDFIKQMELELTLQCSSYQNETQSLKQKCKAFIFPPLQIENWKSATQGLKEILFL

SEQ ID NO: 7 - DNA, 3474 bp, Spatholobus suberectus
  source 1..3474: mol_type = genomic DNA
atggaagcttcatcttcttcccacgttcctcgaatcaaatatgatgtcttcattagctttagaggcacagacgtacggtttggttttcttagccatttgaagaagggactgcgtcagaagcaagttgatgcttttgtggacgacagacttgaaggaggggatgagatatcactctcacttgtcaaagccattgaaggatcacatatttctttggtcatattctccaaagactatgcttcttcaaagtggtgcttggaagagcttgtgaagatcattgagtgcatggacagaaataaacagattgtgattcctgttttctacaacgtagatccatcgcacgtacggcaccaaaaagggacttatggagatgcactagctaaacatgaaagaaataaaagaaacctagccaaggtgccaaattggagatatgctctaaatatagctgctaatctttctggatttcattcgttaaaatttggggatgaagttgagcttatagaagaaattgcgaaatatttgtcgtccaagttgaatcttatgtaccaaagtgagctgacagagcttgtcggaattgaagaacggattgcagatttggagtcactattgtgcctaggatcgacaatagatgttcgtgtgatcgggatttggggcatgggtggtattggaaagacaactattgcagctgcagtttataatcgcctttgttgtgaatatgaagggtgttgctttatggcaaacattacagaacaatcagagaaacatggaatgatttatctgaagaataaaatcatctctatattactgaaggaaaatgatctacatattggcacacctaatggagttcccccatatgttaaaagaaggttggttcgtaaaaaggttctcgttgttcttgacgatatcaatgattcagaacagctagaaaatttagttggagcacttgattggtttggatctggtagcagaatcattgtaacaactagagataagggtgtgctaggaaaaagggttgaccttatatatgaggctaaggcattaaactctgatgaagctattaaaattttcatgttgaatgccttcaaacaaagttgtcttgacttggagtggattgagctatctagaagggtgattcagtatgctaatggaaacccattagccttaaaagtgttgggttcttttctttatggtaagagcaaaatagaatgggaaagccaattacagaaactcaagaaaatgcctcatgcaaaaattcagaatgtattgagattgacttatgatagacttgatcgagaagagaagaacatatttctatatattgcgtgttttttgaaaggatatgaaatacgcaggataatatatctgcttgatgcttgtggtctctcaacaattattgggttaaaagtccttaaagacaaggccctcataattgaagccaagggttctggaatgtcaattgtatcgatgcatgacttgatacaagaaatgggttgggagattgttcgagaagaatgcattgaagaccctggaaaacgaactagattatgggatcctaatgacattcatctagtcttgaaaaataacacgggaactaaagccatcaaaagcataaacttcaacgtctcaaaatttgatgaggtgtgcttaagtcctcaagtctttgcaaagatgcaacagttaaaatttctcaattttacccaacattatggcgatgagcaaatcctgtatcttcccaagggcctcgagtctttgcctaatgacctaagactttttcattgggttagttatcctttgaaattcttgcctcaatcatttcgtgcagaaaatctcgttgagctcaaaatgacttggagccgcgtggaaaaactctggggtgggacccagaatcttgaacatttaaagaaaattgacctcagtaattccaaatatctgctagagctcccagatttttcaaaggcctcaaatcttgaagaggtggaactctatggttgcaaaaacttgcgtaatgttcatccctctattttatctctcaacaagcttgtacggttgaacctattttactgcaaagcgcttactagtctcacaagtgattcccatttaagatctctacgtgacctcttcctgggtggttgctcaagactaaaggagttttcagtgacttcagagaacatgaaagacttaaatttaacttcaacggctatcaatgggttgccctcatcaattgggactctcagaaaacttgaaacactgactcttaatctctgcaaaagtctaaacaatcttccaaacaaggttgctaatctaggatcacttcggtctcttgatatttatggctgcacacaacttgatgcatcaaatctgcatatcctagtcaatggtttaaggtctttggaaacattgaagttggaagaatgccgtaacttatttgaaattcctgataacatcagcctcctatcatcattgcggcagttgttgctaaaaggtacagatattgagagagttcctgcaagcatcaagcatctttcaaagctggaaaagcttgacttaagtggttgcagaaggctttattctctgccagagcttccactgtccataaaagagttatctgccactaactgttcatccttggagactgtaatgttcactttgagtgctgttgaaatactacaagcgtacaagatgcacacaaaattccaaaattgtgttaagttggatgaaccttcactaagtgctattggagtaaatgctcatgtgaacataaagaaagtggcatatgatcatctctcaacaataggaaccaagtttcttgatgctccagttgatgttatctaccctggaattgaggttccagattggttcatgtataggagtacacaggcttcagtaactattgatctctcttctgccccacgatccaagatgatgggtttcatcttttgtgttattgttgataagttcccttcaaacgacaagaactttattggatgtgactgttacatggaaactggtattggtgaaggggtcacacgtgggcacatggatacatggtctagtatacatgcatgtgaatttttctccgaccatgtgtgtctgtggtatgatgaaaaatgctgtctgcaaaaccgtgaatgtgaaagtgaaagcatggaagaacttgtgaccagttataatccaaagattacatttgaattctttgctcaaactggaagtatttgggaaaagcggaaggatattatgattagggggtgtggagtgtgtccaatatacgacacagaatatgacaatttcattgaacagatggaactggagttggaattgacattgcaatctatagcaactaaaatgtcaggtgcaagctctagtaaggaagaaacactcgctccaaaactgcagtgcaagaaactaagctttccaccccatcaaattggaacttggaagagtgcaacacagggcttgaaagatatactgttcctatga

SEQ ID NO: 8 - protein, 1157 aa, Spatholobus suberectus
MEASSSSHVPRIKYDVFISFRGTDVRFGFLSHLKKGLRQKQVDAFVDDRLEGGDEISLSLVKAIEGSHISLVIFSKDYASSKWCLEELVKIIECMDRNKQIVIPVFYNVDPSHVRHQKGTYGDALAKHERNKRNLAKVPNWRYALNIAANLSGFHSLKFGDEVELIEEIAKYLSSKLNLMYQSELTELVGIEERIADLESLLCLGSTIDVRVIGIWGMGGIGKTTIAAAVYNRLCCEYEGCCFMANITEQSEKHGMIYLKNKIISILLKENDLHIGTPNGVPPYVKRRLVRKKVLVVLDDINDSEQLENLVGALDWFGSGSRIIVTTRDKGVLGKRVDLIYEAKALNSDEAIKIFMLNAFKQSCLDLEWIELSRRVIQYANGNPLALKVLGSFLYGKSKIEWESQLQKLKKMPHAKIQNVLRLTYDRLDREEKNIFLYIACFLKGYEIRRIIYLLDACGLSTIIGLKVLKDKALIIEAKGSGMSIVSMHDLIQEMGWEIVREECIEDPGKRTRLWDPNDIHLVLKNNTGTKAIKSINFNVSKFDEVCLSPQVFAKMQQLKFLNFTQHYGDEQILYLPKGLESLPNDLRLFHWVSYPLKFLPQSFRAENLVELKMTWSRVEKLWGGTQNLEHLKKIDLSNSKYLLELPDFSKASNLEEVELYGCKNLRNVHPSILSLNKLVRLNLFYCKALTSLTSDSHLRSLRDLFLGGCSRLKEFSVTSENMKDLNLTSTAINGLPSSIGTLRKLETLTLNLCKSLNNLPNKVANLGSLRSLDIYGCTQLDASNLHILVNGLRSLETLKLEECRNLFEIPDNISLLSSLRQLLLKGTDIERVPASIKHLSKLEKLDLSGCRRLYSLPELPLSIKELSATNCSSLETVMFTLSAVEILQAYKMHTKFQNCVKLDEPSLSAIGVNAHVNIKKVAYDHLSTIGTKFLDAPVDVIYPGIEVPDWFMYRSTQASVTIDLSSAPRSKMMGFIFCVIVDKFPSNDKNFIGCDCYMETGIGEGVTRGHMDTWSSIHACEFFSDHVCLWYDEKCCLQNRECESESMEELVTSYNPKITFEFFAQTGSIWEKRKDIMIRGCGVCPIYDTEYDNFIEQMELELELTLQSIATKMSGASSSKEETLAPKLQCKKLSFPPHQIGTWKSATQGLKDILFL

SEQ ID NO: 9 - DNA, 3480 bp, Cajanus cajan
  source 1..3480: mol_type = genomic DNA
atggcatcgaattcatcttcttcccaagatcctcctcgaatcaaatatgatgtcttcatcagcttcagaggcacagacgtacgatgtggttttcttagccatttgaaaaaggaactgcgtcagaagcaggttgatgcctttgtggatgacaggcttgaaggaggggatgagatatcactctctcttgtcaaagccattgaagggtcgcttatttccttggtcatattctccaaagactatgcttcttcaaagtggtgcttggaagagcttgtgaaaatagttgagtgcatggacataaataaacagattgtgattcctgttttctacaacgttgatccatcggacgtacggcaccaaaaggggacttatggagatgcacttgctaagcatgaaagaagtaaaagaaaccaagccaaggtgcaaagttggagatatgctctaaatataactgctaatctttctggatttcattcatccaaatatggggatgaagttgagcttatagaagaaattgtgaaatgtttgtcgaccaagttgaatctcatgtaccaaagtgagttaacagagcttgtaggaattgaagaacggattgcagatttggagtcagtgttgtgcctaggatcgacaatagatgttcgtgtcattggaatttggggcatgggaggtatcggaaagacaactattgcagctgcactatataaccggctttgttttgaatatgaagggtgttgctttatggcaaacataacggaagaatcagagaaacatggaatgatttatctgaagaataaaatcatctctatattacttaaggaaaatgatctacatattggcacacctaatggagttcccccttatgttaagagaaggttggttcgtaaaaaggttttcgttgttcttgatgatataagtgattccgaacaactagaaaatttggtcggagcccttgattggtttggatctggtagcagaatcattgtaacaactagagataagggtgtgctaggaaaaagggtcgacattgtgtatgaggctaaggcattaaaatatgatgaagctattaaacttttcatgatgaatgccttcaaacaaagttgtcttgacttagaatggattgagctatcaagaagggtgattcagtatgctaatggaaatccattagtcttaaaagtgttgggttcttttctttatggcaagagcaagttagaatgggaaagccaattagagaaacttaagaaaatgcctcatgcaaaaattcagaatgtattgagattgacttatgatagacttgatcgagaagagaagaacatatttctatatattgcatgttttctgaaaggatatgaaatacgcaggataatatatctgcttgatgcttgtggtttctcaacaattattgggttaaaagtccttaaagacaaggccctcataattgaagccaagggctctggaatctcaattgtatcaatgcatgacttgatacaagaaatgggttgggagatagttcgagaagaatgcattgaagaccctggaaaacgaacaaggttatgggatcctaatgacattcatctagtattgaaaaataacacgggaactaaagccatcaagagcataactttcaacctctcaaaatttgatgaggtgtgctcaagtcctcaagtgttcgcaaagatgcaacagttaaaatttcttaatttttcccaacattatggcgatgagcaaatcctgtatcttcccaagggcctagaatctttgccgaatgacctaaggctttttcattgggttagttatcctttgaaatccttgcctcaatcattttgtgcagaaaatctcattgagctcaaaatgccgtggagccgagtggaaaaactttgggatgggatccagaatcttgaacatttaaagaaaattgacctcagtaattccaaaaatttgctagaggtcccagatttttcaaaggcctcaaatcttgaagaggtggaactctctgcatgcgaaaacttgcgcaatgttcatccatctattttatctctcaacaagcttgtaaggttgaacttatttgactgcaaagcacttaccagtctcagaagtgattcccatttaagatctctacgtgacctcttcctcggtggttgctcaagactccaggagttttcagtgagttcagagaacatgaaaaaattaattttaacttcaaccgcaatcagtgagttgccctcatcagttggaactctcggaaaacttgaaacattgatccttgataactgcaaaagtctgaacaatcttccaaacaaggttgcttacctgagatcacttcggtcacttgatatttatggctgcacacaacttgatgcatcaaatcttcatatcctagttaatggtttaaggtcgctggaatcattgaagttggaagaatgccgcaacttatttgaaattccagataacatcaacctcctatcatcattgaggcagctgttgctaaaaggaacagatattgaaagagttcctttaagcatcaagcatctttctaatctggaaaagcttgacttaagtaattgcaaaaggctttattctttgccagagcttccactatccataaaagagttttatgccactaactgttcatccttggagaatgtaatgttcactttgagtgctgttgaaatactaaaagcatacaagatgcacacaaaattccaaaactgtgtgaagttggatgaacattcactcagtgctattggagtaaatgctcatgtgaacataaagaaagtggcatatgatcatctctcaacgataggaaccaagtttcttgatggtccagttgatgttatctaccctggaagtaaggttccagagtggttcttgtatagaactacacgggcttcggtaactattgatttctcttctgctccatgttccaagatcatgggattcatcttttgtgttattgttgataagttcccttcaaatgacaagaacttcattggatgtgattgttacatggaaactggtattggtgaaagtgtcacacgtgggcacatggatacgtggtctagtatacatgcatgtgaatttttctctgaacatgtgtgtctgtggtatgatgaaaaatgttgtctgcaaaacagtgaacgtgaaagtgaaaacatggaagaacttatggccagttataatccaaagatttcatttgaattttttgctcaaactggaagtatttgggaaaagcgaagggatattatgattaaagggtgtggagtgtgtccaatatacgacacagaatatgacaatttcattatgcagatggaattggagttggaattgacgttgcaatctatagcaactaaaatgtcagaagcatgctctagtaaggatgaaaccgtctctccaaaacatcaatgcaagaaactaagctttccaccctatcaaattggaacttggaagagtgcaacacaaggcttgaaagatatattgttcctgtaa

SEQ ID NO: 10 - protein, 1159 aa, Cajanus cajan
MASNSSSSQDPPRIKYDVFISFRGTDVRCGFLSHLKKELRQKQVDAFVDDRLEGGDEISLSLVKAIEGSLISLVIFSKDYASSKWCLEELVKIVECMDINKQIVIPVFYNVDPSDVRHQKGTYGDALAKHERSKRNQAKVQSWRYALNITANLSGFHSSKYGDEVELIEEIVKCLSTKLNLMYQSELTELVGIEERIADLESVLCLGSTIDVRVIGIWGMGGIGKTTIAAALYNRLCFEYEGCCFMANITEESEKHGMIYLKNKIISILLKENDLHIGTPNGVPPYVKRRLVRKKVFVVLDDISDSEQLENLVGALDWFGSGSRIIVTTRDKGVLGKRVDIVYEAKALKYDEAIKLFMMNAFKQSCLDLEWIELSRRVIQYANGNPLVLKVLGSFLYGKSKLEWESQLEKLKKMPHAKIQNVLRLTYDRLDREEKNIFLYIACFLKGYEIRRIIYLLDACGFSTIIGLKVLKDKALIIEAKGSGISIVSMHDLIQEMGWEIVREECIEDPGKRTRLWDPNDIHLVLKNNTGTKAIKSITFNLSKFDEVCSSPQVFAKMQQLKFLNFSQHYGDEQILYLPKGLESLPNDLRLFHWVSYPLKSLPQSFCAENLIELKMPWSRVEKLWDGIQNLEHLKKIDLSNSKNLLEVPDFSKASNLEEVELSACENLRNVHPSILSLNKLVRLNLFDCKALTSLRSDSHLRSLRDLFLGGCSRLQEFSVSSENMKKLILTSTAISELPSSVGTLGKLETLILDNCKSLNNLPNKVAYLRSLRSLDIYGCTQLDASNLHILVNGLRSLESLKLEECRNLFEIPDNINLLSSLRQLLLKGTDIERVPLSIKHLSNLEKLDLSNCKRLYSLPELPLSIKEFYATNCSSLENVMFTLSAVEILKAYKMHTKFQNCVKLDEHSLSAIGVNAHVNIKKVAYDHLSTIGTKFLDGPVDVIYPGSKVPEWFLYRTTRASVTIDFSSAPCSKIMGFIFCVIVDKFPSNDKNFIGCDCYMETGIGESVTRGHMDTWSSIHACEFFSEHVCLWYDEKCCLQNSERESENMEELMASYNPKISFEFFAQTGSIWEKRRDIMIKGCGVCPIYDTEYDNFIMQMELELELTLQSIATKMSEACSSKDETVSPKHQCKKLSFPPYQIGTWKSATQGLKDILFL

SEQ ID NO: 11 - DNA, 3465 bp, Mucuna pruriens
  source 1..3465: mol_type = genomic DNA
atggaagtgaattcatgttcttccgacgttcctcgaataaaatatgatgtattcattagctttagaggcacagacgtacgatgtggttttcttagccatttgaaaaaggaactgcgtcagaagcaggttgatgcctttgtggatgacagacttgaaggaggggatgagatatcacactcacttgtcaaagccattgaaggatcagttatttctttggtcatattctccaaagactatgcttcttcaaagtggtgcttggaagagcttgtgaaaataattgagtgcatggacagatttaaacagattgttattcctgttttctacaacgtagatccatccaacgtacggcaccaaaaagggacttatggagatgctttggctaaacatgaaacaaataaaagaaacctagccaaggtgcaaaattggaaatgtgctctaaatattgctgctaatctttctggatttcattcattaaaatttggggatgaagttgagcttatagaagagattgtcaaatgtttgtcttccaagttgaatcttatgtaccaaagtgagttaacagagcttgttggaattgaagaacgaatttcagaatttgagtcactattgtgcctaggatcgacaatagatgttcgtgtcattgggatatggggcatgggtggtattggaaagacaactattgcagctgcagtatataaccgcctttgttttgaatatgaagggtgttgctttatggcaaacataacagaagaatcagagaaacatggaatgatttatctgaagaataaaattatctctattttactgaaggaaaatgatctacatattggcacacctaatggagttcccccatatgttaagagaaggttggttcgtaaaaaggttttcgttgttcttgatgatatcaatgattcggaacaactagaaaatttagtcggagcacttgattggtttggatctggtagcagaatcattgtaacaactagagataagggtgtgctaggaaaaaaggttgacattatatatgaggctaaggcattaaactctgatgaagctattaaacttttcatgttgaatgccttcaaacaaagttgtcttgacatggagtggattgagctatcgagaagggtgattctgtatgctaatggaaacccattagccttaaaagtgttgggttcttttctttatggtaagagcaaaatagaatgggaaagccaattacagaaacttaagaagatgcctcatgcaaaaattcagaatgtattgagattgacttatgatagacttgatagagaggagaagaacatatttctatatattgcgtgttttttgaaaggatatgaaatacgtcgaataatatatctgcttgatgcttgtggcttctcaacaattattgggttaaaagtccttaaagacaaggccctcataattgaagccaagggctctggaatctcaattgtatcaatgcatgacttgatacaagaaatgggttgggaaattgttcgagaagaatgcattgaagaccctggaaaacgaactaggttatgggatcctaatgacattcatctagtattgaaaaataacacgggaactaaagccatcaaaagcataacttttaatgtctcaaaatttgatgaggtgtgcttaagtcctcaagtctttgcaaggatgcagcacttaaaatttcttaattttacccaacattacggcgacgagcaaatcctgtatcttcccaagggcctcgagtctttgcctaataacctaagacttcttcattgggttagttatcctctgaaatccttgcctcaatcattttgtgcagaaaatctggttgagctcaaaatgacttggagccgagtggaaaaactctgggatgggatccagaatcttgaacatttaaagaaaattgacctcagtaattccaaaaatctgctagagctcccagatttttcaaaggcttcaaatcttgaagaggtggatctccatgcttgcaaaaacttgcgtaatgttcatccatctattttatctctcaaaaaacttgtgcggttgaacctattttactgcaaagcgcttaccattcttagaagtgattcccatttaagatccctacgcgccctcttcctcggtggttgctcaagactgaaggagttttcagtgacttcagagaacatgaaagacttaaatttaacttcaacggcgatcaaagagttgccctcatcaattgggagtctcagaaagctggaaacacttactcttgatcactgtcaaagtctaaacaatcttccaaacaaggttgctaacctaagatcacttcggtctcttgatatttatggctgcacacaacttgatgcatcaaatctgcatatcctagttaatggtttaaggtctttggaaacattgaagttggaagaatgccgtaacttatttgaaattccagataacatcaacctcctctcattgttgcggcacttgttgctaaaaggaacagatattgagagagttcctgcaagcatcaagcatctttccaagctggaaaagcttgacttaagtggttgcaaaaggctttattctctgccagaacttccaccttccataaaagagttatatgccactaactgttcatccttagagactgtaatgttcactttgagtgttgaaatactacaagcagcatacaaggtgcacacaaatttccaaaattgtgtgaagttggatgaacattcactcagtgctattggagtaaatgctcatgtgaacataaagagagtggcatatgataatctctcaacaataggaagcaagtttcttgatgggccagttgatgttatctaccctggaagtgagattccagagtggtttgtgtataggactacacatgcttcagtaactattgatctctcttccgctccatgttccaagatcatgggtttcatcttttgtgttattgttgatgagttcccttcaaacgacaagaacttcatcgggtgtgactgttacatggaaactggtgttggtgaaagggtgacacgtgggcacatggataattggtctagtatacatgcctgtgaaattttgtccgaccatgtgtgtctgtggtacgatgaaaaatgttgtctgcaaaaccgtgaatgtgaaagagaaagcatggaggaacttatggccagttataatccaaagatttcatttgaattctttgctcaaactggaagtatctgggaaaagcggaaggatattatgatcaaagggtgtggagtgtgtccaatttacgacacagaatatggcaatttcattaaacagatggaattgacgttgcaatcattagcaactaaaatgtcgagtgcaagctctactaagggagaaccaatcgctccaaaacagcaatgcaagaaactaatctttccaccccttcaaattggaacttggaagaatgcaacacaaggcttgaaagatatactgttcctgtga

SEQ ID NO: 12 - protein, 1154 aa, Mucuna pruriens
MEVNSCSSDVPRIKYDVFISFRGTDVRCGFLSHLKKELRQKQVDAFVDDRLEGGDEISHSLVKAIEGSVISLVIFSKDYASSKWCLEELVKIIECMDRFKQIVIPVFYNVDPSNVRHQKGTYGDALAKHETNKRNLAKVQNWKCALNIAANLSGFHSLKFGDEVELIEEIVKCLSSKLNLMYQSELTELVGIEERISEFESLLCLGSTIDVRVIGIWGMGGIGKTTIAAAVYNRLCFEYEGCCFMANITEESEKHGMIYLKNKIISILLKENDLHIGTPNGVPPYVKRRLVRKKVFVVLDDINDSEQLENLVGALDWFGSGSRIIVTTRDKGVLGKKVDIIYEAKALNSDEAIKLFMLNAFKQSCLDMEWIELSRRVILYANGNPLALKVLGSFLYGKSKIEWESQLQKLKKMPHAKIQNVLRLTYDRLDREEKNIFLYIACFLKGYEIRRIIYLLDACGFSTIIGLKVLKDKALIIEAKGSGISIVSMHDLIQEMGWEIVREECIEDPGKRTRLWDPNDIHLVLKNNTGTKAIKSITFNVSKFDEVCLSPQVFARMQHLKFLNFTQHYGDEQILYLPKGLESLPNNLRLLHWVSYPLKSLPQSFCAENLVELKMTWSRVEKLWDGIQNLEHLKKIDLSNSKNLLELPDFSKASNLEEVDLHACKNLRNVHPSILSLKKLVRLNLFYCKALTILRSDSHLRSLRALFLGGCSRLKEFSVTSENMKDLNLTSTAIKELPSSIGSLRKLETLTLDHCQSLNNLPNKVANLRSLRSLDIYGCTQLDASNLHILVNGLRSLETLKLEECRNLFEIPDNINLLSLLRHLLLKGTDIERVPASIKHLSKLEKLDLSGCKRLYSLPELPPSIKELYATNCSSLETVMFTLSVEILQAAYKVHTNFQNCVKLDEHSLSAIGVNAHVNIKRVAYDNLSTIGSKFLDGPVDVIYPGSEIPEWFVYRTTHASVTIDLSSAPCSKIMGFIFCVIVDEFPSNDKNFIGCDCYMETGVGERVTRGHMDNWSSIHACEILSDHVCLWYDEKCCLQNRECERESMEELMASYNPKISFEFFAQTGSIWEKRKDIMIKGCGVCPIYDTEYGNFIKQMELTLQSLATKMSSASSTKGEPIAPKQQCKKLIFPPLQIGTWKNATQGLKDILFL

SEQ ID NO: 13 - DNA, 3477 bp, Glycine soja
  source 1..3477: mol_type = genomic DNA
atggaaacttcttctagttcccaagatcctcgaatcaaatatgatgtcttcatcagctttagaggcacagacgtacggtgtggttttcttagccatttgaagaaggaactgcgccagaagcaagttgatgcctttgttgatgacaggcttgaaggaggggatgagatatcacactcacttgacaaagccattgaaggatcacttatttctttggtcatattctccaaagactatgcttcttcaaagtggtgcttggaagaggttgtgaaaataattgagtgcatgcacagtaataaacagattgtgattcctgttttctacaacgtagatccatcagacgtacggcaccaaaaagggacttatggagatgcatttgctaagcacgaaaaaaataaaagaaaccttgcaaaggtgccaaattggagatgtgctctaaatatagctgccaatctttctggatttcattcatcaaaatttgtggatgaagttgagcttatagaagaaattgccaaatgtttgtcatccaagttgaatcttatgtaccaaagtgagttaacagagcttgttggaattgaagaacggattgcagatttggagtcactattgtgcctaggatcgacaatagtaggtgttcgtgtcattgggatttggggcatgggtggtattggaaagacaactattgcagctgcagtatataaccgcctttattttgaatatgaagggtgttgctttatggcaaacataacagaagaatcagagaagcatggaatgatttatgtgaagaacaaaattatctctatactactaaaggaaaatgatctacagattggcacacctaatggagttcccccatatgttaagagaaggttgattcgtaaaaaggttctcgttgttcttgatgatatcaatgattcagaacaactagaaaatttagttggagcacttgattggtttggatctggtagtagaatcattgtaacaactagagataagggtgtgcttgggaaaaaggctgatattgtatatgaggctaaggcattaaactctgatgaagctattaaacttttcatgttgaatgccttcaaacaaagttgtcttgagatggagtggattgagctttcgagaagggtgattcagtatgctaatggaaacccattggccttaaaagtgttgggttcttttctttatggcaagagccaaatagaatgggaaagccaattacagaaacttaagaaaatgcctcatgtaaaaattcagaatgtattgagattgacttatgatagacttgatcgagaagagaagaacatatttctatatattgcatgtttttttaaaggatatgaagtacgccgaataatatatctgcttgatgcttgtggtttctctacaattattgggttaagagtccttaaagacaaggccctcataattgaagccaagggctctggaatctcaattgtatcaatgcatgacttgatacaagaaatgggctgggagattgttcgagaagaatgcatcgaagaccctggaaaacgaactaggttatgggatcctaatgacattcatctagtgttgaaaaataacacgggaactaaagccatcaaaagcataacttttaacgtctcaaaatttgatgaggtgtgcttaagtcctcaaatctttgaaaggatgcaacagttaaaatttcttaattttacccaacattatggcgatgagcaaatcctgtatcttcccaaaggccttgaatctttgcctaatgacctaagactttttcattgggttagttatcctttgaaatccttgcctctatcattttgtgctgaaaatctcgttgagctcaaattgccttggagccgagtggaaaaactttgggatggaatccagaatcttgaacatttaaagaaaattgacctgagttattccaaaaatctgctagagctcccagatttttcaaaggcctcaaatcttgaagaggtggaactctatagttgcaaaaacttgcgtaatgttcatccatctattttatctctcaaaaagcttgtacggttgaatctcttctactgcaaagcgcttaccagtcttagaagtgattcccatttaagatctctacgtgacctcttcctgggtggttgctcaagactcaaggagttttcagtgacttcagagaatatgaaagacttaattttaacttcaactgctatcaacgagttgccctcatcgattgggagtctaagaaaactagaaacactgacccttgatcactgcaaaagcctaagcaatcttccaaacaaggttgccaacctaagatcacttcgacgtcttcatatttatggctgcacacaacttgatgcatcaaatcttcatatcctagttaatggtttaaagtctttggaaacattgaagttggaagaatgccgtaacttgtttgaaattccagacaacatcaacctcctatcatcattgcgtgagttgttactaaaaggaacagatattgagagtgtttctgcaagcatcaagcatctttcaaagctggaaaagcttgacttgagtgattgcagaaggctttattctctgccagagcttccacagtccataaaagagctatatgccattaactgttcatccttggagactgtaatgttcactttgagtgctgttgaaatgctacatgcatacaagttacacacgacattccaaaattgtgtaaaattggatcaacattcactcagtgctattggagtaaatgcttatgtgaacataaagaaagtggcatatgatcaattttcaacaattggaaccaactcaatcaagtttcttggtggtccagttgattttatctaccctggaagtgaggttccagagtggttcgtgtataggactacacaggcttcagtaactgttgatctctcttcttctgttccatgttccaagatcatgggtttcatcttttgtgttattgttgatcagttcacttcaaacgacaagaactatattggatgtgactgttacatggaaaccggtgttggtgaaagggtcacacgtgggcacatggataattggtctagcatacatgcctgtgaatttttctccgaccatgtgtgtctgtggtatgatgaaaaatgctgtctgaaaaaccaagaatgtgaaagtgaaagcatggaagaacttatggcaagttataatccaaagatttcatttgaattctttgctaaaactggaagtatttgggaaaagcggagcgatattataattaaagggtgcggagtgtgtccaatatatgacacagaatgtgacaatttctttaaacagatggaattggagttggaaatcacattgcaatccatggcaactaaaatgtcaagtaaggaagcaacactatctccaaaacaggaatctaagaaactgatctttccaccccatcaaattggaacttggaagaatgcaacacaagggttgaaagatatactgttcctgtga

SEQ ID NO: 14 - protein, 1158 aa, Glycine soja
METSSSSQDPRIKYDVFISFRGTDVRCGFLSHLKKELRQKQVDAFVDDRLEGGDEISHSLDKAIEGSLISLVIFSKDYASSKWCLEEVVKIIECMHSNKQIVIPVFYNVDPSDVRHQKGTYGDAFAKHEKNKRNLAKVPNWRCALNIAANLSGFHSSKFVDEVELIEEIAKCLSSKLNLMYQSELTELVGIEERIADLESLLCLGSTIVGVRVIGIWGMGGIGKTTIAAAVYNRLYFEYEGCCFMANITEESEKHGMIYVKNKIISILLKENDLQIGTPNGVPPYVKRRLIRKKVLVVLDDINDSEQLENLVGALDWFGSGSRIIVTTRDKGVLGKKADIVYEAKALNSDEAIKLFMLNAFKQSCLEMEWIELSRRVIQYANGNPLALKVLGSFLYGKSQIEWESQLQKLKKMPHVKIQNVLRLTYDRLDREEKNIFLYIACFFKGYEVRRIIYLLDACGFSTIIGLRVLKDKALIIEAKGSGISIVSMHDLIQEMGWEIVREECIEDPGKRTRLWDPNDIHLVLKNNTGTKAIKSITFNVSKFDEVCLSPQIFERMQQLKFLNFTQHYGDEQILYLPKGLESLPNDLRLFHWVSYPLKSLPLSFCAENLVELKLPWSRVEKLWDGIQNLEHLKKIDLSYSKNLLELPDFSKASNLEEVELYSCKNLRNVHPSILSLKKLVRLNLFYCKALTSLRSDSHLRSLRDLFLGGCSRLKEFSVTSENMKDLILTSTAINELPSSIGSLRKLETLTLDHCKSLSNLPNKVANLRSLRRLHIYGCTQLDASNLHILVNGLKSLETLKLEECRNLFEIPDNINLLSSLRELLLKGTDIESVSASIKHLSKLEKLDLSDCRRLYSLPELPQSIKELYAINCSSLETVMFTLSAVEMLHAYKLHTTFQNCVKLDQHSLSAIGVNAYVNIKKVAYDQFSTIGTNSIKFLGGPVDFIYPGSEVPEWFVYRTTQASVTVDLSSSVPCSKIMGFIFCVIVDQFTSNDKNYIGCDCYMETGVGERVTRGHMDNWSSIHACEFFSDHVCLWYDEKCCLKNQECESESMEELMASYNPKISFEFFAKTGSIWEKRSDIIIKGCGVCPIYDTECDNFFKQMELELEITLQSMATKMSSKEATLSPKQESKKLIFPPHQIGTWKNATQGLKDILFL

SEQ ID NO: 15 - DNA, 3477 bp, Glycine max
  source 1..3477: mol_type = genomic DNA
atggaaacttcttctagttcccaagatcctcgaatcaaatatgatgtcttcatcagctttagaggcacagacgtacggtgtggttttcttagccatttgaagaaggaactgcgccagaagcaagttgatgcctttgttgatgacaggcttgaaggaggggatgagatatcacactcacttgacaaagccattgaaggatcacttatttctttggtcatattctccaaagactatgcttcttcaaagtggtgcttggaagaggttgtgaaaataattgagtgcatgcacagtaataaacagattgtgattcctgttttctacaacgtagatccatcagacgtacggcaccaaaaagggacttatggagatgcatttgctaagcacgaaaaaaataaaagaaaccttgcaaaggtgccaaattggagatgtgctctaaatatagctgccaatctttctggatttcattcatcaaaatttgtggatgaagttgagcttatagaagaaattgccaaatgtttgtcatccaagttgaatcttatgtaccaaagtgagttaacagagcttgttggaattgaagaacggattgcagatttggagtcactattgtgcctaggatcgacaatagtaggtgttcgtgtcattgggatttggggcatgggtggtattggaaagacaactattgcagctgcagtatataaccgcctttattttgaatatgaagggtgttgctttatggcaaacataacagaagaatcagagaagcatggaatgatttatgtgaagaacaaaattatctctatactactaaaggaaaatgatctacagattggcacacctaatggagttcccccatatgttaagagaaggttgattcgtaaaaaggttctcgttgttcttgatgatatcaatgattcagaacaactagaaaatttagttggagcacttgattggtttggatctggtagtagaatcattgtaacaactagagataagggtgtgcttgggaaaaaggctgatattgtatatgaggctaaggcattaaactctgatgaagctattaaacttttcatgttgaatgccttcaaacaaagttgtcttgagatggagtggattgagctttcgagaagggtgattcagtatgctaatggaaacccattggccttaaaagtgttgggttcttttctttatggcaagagccaaatagaatgggaaagccaattacagaaacttaggaaaatgcctcatgtaaaaattcagaatgtattgagattgacttatgatagacttgatcgagaagagaagaacatatttctatatattgcatgtttttttaaaggatatgaagtacgccgaataatatatctgcttgatgcttgtggtttctctacaattattgggttaagagtccttaaagacaaggccctcataattgaagccaagggctctggaatctcaattgtatcaatgcatgacttgatacaagaaatgggctgggagattgttcgagaagaatgcatcgaagaccctggaaaacgaactaggttatgggatcctaatgacattcatctagtgttgaaaaataacacgggaactaaagccatcaaaagcataacttttaacgtctcaaaatttgatgaggtgtgcttaagtcctcaaatctttgaaaggatgcaacagttaaaatttcttaattttacccaacattatggcgatgagcaaatcctgtatcttcccaaaggccttgaatctttgcctaatgacctaagactttttcattgggttagttatcctttgaaatccttgcctctatcattttgtgctgaaaatctcgttgagctcaaattgccttggagccgagtggaaaaactttgggatggaatccagaatcttgaacatttaaagaaaattgacctgagttattccaaaaatctgctagagctcccagatttttcaaaggcctcaaatcttgaagaggtggaactctatagttgcaaaaacttgcgtaatgttcatccatctattttatctctcaaaaagcttgtacggttgaatctcttctactgcaaagcgcttaccagtcttagaagtgattcccatttaagatctctacgtgacctcttcctgggtggttgctcaagactcaaggagttttcagtgacttcagagaatatgaaagacttaattttaacttcaactgctatcaacgagttgccctcatcgattgggagtctaagaaaactagaaacactgacccttgatcactgcaaaagcctaagcaatcttccaaacaaggttgccaacctaagatcacttcgacgtcttcatatttatggctgcacacaacttgatgcatcaaatcttcatatcctagttaatggtttaaagtctttggaaacattgaagttggaagaatgccgtaacttgtttgaaattccagacaacatcaacctcctatcatcattgcgtgagttgttactaaaaggaacagatattgagagtgtttctgcaagcatcaagcatctttcaaagctggaaaagcttgacttgagtgattgcagaaggctttattctctgccagagcttccacagtccataaaagagctatatgccattaactgttcatccttggagactgtaatgttcactttgagtgctgttgaaatgctacatgcatacaagttacacacgacattccaaaattgtgtaaaattggatcaacattcactcagtgctattggagtaaatgcttatgtgaacataaagaaagtggcatatgatcaattttcaacaattggaaccaactcaatcaagtttcttggtggtccagttgattttatctaccctggaagtgaggttccagagtggttcgtgtataggactacacaggcttcagtaactgttgatctctcttcttctgttccatgttccaagatcatgggtttcatcttttgtgttattgttgatcagttcacttcaaacgacaagaactatattggatgtgactgttacatggaaaccggtgttggtgaaagggtcacacgtgggcacatggataattggtctagcatacatgcctgtgaatttttctccgaccatgtgtgtctgtggtatgatgaaaaatgctgtctgaaaaaccaagaatgtgaaagtgaaagcatggaagaacttatggcaagttataatccaaagatttcatttgaattctttgctaaaactggaagtatttgggaaaagcggagcgatattataattaaagggtgcggagtgtgtccaatatatgacacagaatgtgacaatttctttaaacagatggaattggagttggaaatcacattgcaatccatggcaactaaaatgtcaagtaaggaagcaacactatctccaaaacaggaatctaagaaactgatctttccaccccatcaaattggaacttggaagaatgcaacacaagggttgaaagatatactgttcctgtga

SEQ ID NO: 16 - protein, 1158 aa, Glycine max
METSSSSQDPRIKYDVFISFRGTDVRCGFLSHLKKELRQKQVDAFVDDRLEGGDEISHSLDKAIEGSLISLVIFSKDYASSKWCLEEVVKIIECMHSNKQIVIPVFYNVDPSDVRHQKGTYGDAFAKHEKNKRNLAKVPNWRCALNIAANLSGFHSSKFVDEVELIEEIAKCLSSKLNLMYQSELTELVGIEERIADLESLLCLGSTIVGVRVIGIWGMGGIGKTTIAAAVYNRLYFEYEGCCFMANITEESEKHGMIYVKNKIISILLKENDLQIGTPNGVPPYVKRRLIRKKVLVVLDDINDSEQLENLVGALDWFGSGSRIIVTTRDKGVLGKKADIVYEAKALNSDEAIKLFMLNAFKQSCLEMEWIELSRRVIQYANGNPLALKVLGSFLYGKSQIEWESQLQKLRKMPHVKIQNVLRLTYDRLDREEKNIFLYIACFFKGYEVRRIIYLLDACGFSTIIGLRVLKDKALIIEAKGSGISIVSMHDLIQEMGWEIVREECIEDPGKRTRLWDPNDIHLVLKNNTGTKAIKSITFNVSKFDEVCLSPQIFERMQQLKFLNFTQHYGDEQILYLPKGLESLPNDLRLFHWVSYPLKSLPLSFCAENLVELKLPWSRVEKLWDGIQNLEHLKKIDLSYSKNLLELPDFSKASNLEEVELYSCKNLRNVHPSILSLKKLVRLNLFYCKALTSLRSDSHLRSLRDLFLGGCSRLKEFSVTSENMKDLILTSTAINELPSSIGSLRKLETLTLDHCKSLSNLPNKVANLRSLRRLHIYGCTQLDASNLHILVNGLKSLETLKLEECRNLFEIPDNINLLSSLRELLLKGTDIESVSASIKHLSKLEKLDLSDCRRLYSLPELPQSIKELYAINCSSLETVMFTLSAVEMLHAYKLHTTFQNCVKLDQHSLSAIGVNAYVNIKKVAYDQFSTIGTNSIKFLGGPVDFIYPGSEVPEWFVYRTTQASVTVDLSSSVPCSKIMGFIFCVIVDQFTSNDKNYIGCDCYMETGVGERVTRGHMDNWSSIHACEFFSDHVCLWYDEKCCLKNQECESESMEELMASYNPKISFEFFAKTGSIWEKRSDIIIKGCGVCPIYDTECDNFFKQMELELEITLQSMATKMSSKEATLSPKQESKKLIFPPHQIGTWKNATQGLKDILFL

SEQ ID NO: 17 - DNA, 3477 bp, Trifolium pratense
  source 1..3477: mol_type = genomic DNA
atggcatcttcatcatcttcacatcatgttcctcaaattaaatatgatgtattcattagctttagaggcacagacatacgtcatggttttttaggccatttgagaaaagaattgaggcaaaagcaagttgatgcctatgtggatgatagacttgaaggaggagaagaaataacacaagcacttgttaaagccattgaaggatcacttatttctttgataatattttccaaggactatgcttcttcaaagtggtgtttggaagagcttgttaaaattattcaatgtatggctagaaataaacaaattgtcatccctgttttctataatgttaatccaacggatgtaaggcaccaaaaagggacttatgctgattcatttgctaaacatgaaataaataatgtaagcttagctaaggtgcaaaattggagatctgctttgactattgcttctaatctttctggatttcattcatcaaaatttggggatgaagttgagcttatagaaaaaattgtcaaatatttgtcatccaagttgaatgttatgtaccaaagtgagttaacagatcttgttggaattgaacaaaggattgcagatttagagtcactgttatgcctagattcaactgtagatgttcttgtcgttggcatttggggtatgggcggtattggcaagacaactcttgcagctgcagtgtataaccgtctttgttttgaatatgaaggatgttgctttatggcaaatattacagaagaatcagagaagcatggaatgatttatctgaagaataaaattctctctatattgctaaatgaaaatcatctacatatcggtacacctaatggagttccaccttatgttaaaagaaggttggttcgtaaaaaagttcttcttgttcttgatgatatcaatgatttagagcatctagagaatttagttggaggacttgattggtttggatccggtagcagaatcatcgtaacaaccagagataagcaagtgcttggtaaaagagtcaattgtatatacgaggccaaggcattggagtctgatgatgctattaaacttttcatcacaaatgccttcaaaaatggttgtcttgacatggaatggattgagctatcaagaagggtgattcattatgctaatggaaatccattagttttgaaagttttgggttcttttctttatgataaaagcaaactagaatgggaaagtcagttgcagaaacttaagaagatgcctcatgcaaaaattcaaaatgtattgagattgagttatgatagactcgatcgagaagagaagaacatatttttatatattgcatgtctgttgaaaggatatgaagttcaacaaataatagctttgcttgatgcttgtggtttctcaacaattattgggttaaaagtccttaaagacaaagcccttataattgaagccaagggcgctggaaaatcaatggttctgatgcatgacttgatacaagaaatgggctgggagattgttcgagaagaatgtattgaagaccctggaaaacgcagcaggttatgggatcctaatgatgttcatcaagtattgacaaataacacgggaactaaagccgtcaaaagcataactctgaatgtctcgaaatttgatgagctgcagttaagtcctcaagtcttcggtaggatgcagcagttaaaatttcttaaatttacccaacattatggcgacgagaaaatactgtatcttcctcagggtcttgaatctttgccaaatgacctactgctttttcaatgggttagttatcctttgaaatccctgcctcagtctttttgtgcagaaaatcttgttgagctaaagttgacttcaagccgagtggaaaaactttgggatggagtccagaatattcaacatttaaagaaaatggacctcagttattccaaatatctgctagagctccctgatttttcaaaggcctcaaatcttgaagaaatagaactctatggctgcaaaagtttgcttaatgttcatccttcaattttacgtctcaacaagctagtgcgactgaacctattttactgcaaagcacttaccagtcttcgaagtgatactcatttaagatcccttcgcgatctattcctcggtggttgctcaaaactcaaggagttttcagtgacatctgagaatatgaaagacttaactttaacttcaacagctatcaatgaattgccctcatcaattggtagtctcaaaaaactcgaatcattgagtcttgatttctgcaaaagcctaatcaaccttccaaatgaaattaccgacctgagatcacttcgagctctttacattcacggttgcacacaacttgatgcatcaaatcttcatatcctaattagtggtttagcttctttagaaacgttaaagctggaagaatgtcgtaacttattcgaaattccagacaacatcagcttcctatcatcattgcgtgagttgttactaaaagaaactgatattgagatatttcctgcaagcatcaagcatctttctaagctggaaaaacttgacataaagggctgcaggaggcttcattatcttccagagcttccgccatcactgaaagagttttatgccactaattgttcatctttagagactgtgatgttcaattggaatgctgctgatctactacaacatcaagcatataagttatatacacgatttgaaaattgtgttaacttggatgaactttcactcagggcaattgaagtaaatgctcaagtaaacatgaagaaattggtatataagaatctctcaacattaggaagcaaatttcttgatggtccagttgatgtgatctaccccggaagtaaagttccagagtggtttatgcacagaacaacacaggcttcagtgactgttgatttctcttccgctccaaattccaagtttgttggtttcatcttctgcgtcattgctggtcagctcccatcggacgataaaaacttcattggatgtgactgttacctggaaactggtaatggcgaaagggtcgctcttgggaacatggatacatggactagtatacattcccccgagtttttttccgaccatgtttttatatggtatgatgaaatgtgttgtctgcaaaacagtgaacccaaaaaggaaaacgtggaagaactcatggccaattataatccaaaggtttcatttgaattctttgctcaaagtggaaatacttgggaaaagagagagagtagtatgataagagggtgtggagtgtgtccaatatatgatacagaatattttgatttcattaaacaaatggaattggagttggaattcacactgcaatccattgctaatgaaaggttagttcaatgcagtgataagaaagaaacactagacccaaaacaaccatgcaagaaattcttttcacacgttcaaactggaatttggaagagtgcaacacaaggcttgaaagatatattgttgtga

SEQ ID NO: 18 - protein, 1158 aa, Trifolium pratense
MASSSSSHHVPQIKYDVFISFRGTDIRHGFLGHLRKELRQKQVDAYVDDRLEGGEEITQALVKAIEGSLISLIIFSKDYASSKWCLEELVKIIQCMARNKQIVIPVFYNVNPTDVRHQKGTYADSFAKHEINNVSLAKVQNWRSALTIASNLSGFHSSKFGDEVELIEKIVKYLSSKLNVMYQSELTDLVGIEQRIADLESLLCLDSTVDVLVVGIWGMGGIGKTTLAAAVYNRLCFEYEGCCFMANITEESEKHGMIYLKNKILSILLNENHLHIGTPNGVPPYVKRRLVRKKVLLVLDDINDLEHLENLVGGLDWFGSGSRIIVTTRDKQVLGKRVNCIYEAKALESDDAIKLFITNAFKNGCLDMEWIELSRRVIHYANGNPLVLKVLGSFLYDKSKLEWESQLQKLKKMPHAKIQNVLRLSYDRLDREEKNIFLYIACLLKGYEVQQIIALLDACGFSTIIGLKVLKDKALIIEAKGAGKSMVLMHDLIQEMGWEIVREECIEDPGKRSRLWDPNDVHQVLTNNTGTKAVKSITLNVSKFDELQLSPQVFGRMQQLKFLKFTQHYGDEKILYLPQGLESLPNDLLLFQWVSYPLKSLPQSFCAENLVELKLTSSRVEKLWDGVQNIQHLKKMDLSYSKYLLELPDFSKASNLEEIELYGCKSLLNVHPSILRLNKLVRLNLFYCKALTSLRSDTHLRSLRDLFLGGCSKLKEFSVTSENMKDLTLTSTAINELPSSIGSLKKLESLSLDFCKSLINLPNEITDLRSLRALYIHGCTQLDASNLHILISGLASLETLKLEECRNLFEIPDNISFLSSLRELLLKETDIEIFPASIKHLSKLEKLDIKGCRRLHYLPELPPSLKEFYATNCSSLETVMFNWNAADLLQHQAYKLYTRFENCVNLDELSLRAIEVNAQVNMKKLVYKNLSTLGSKFLDGPVDVIYPGSKVPEWFMHRTTQASVTVDFSSAPNSKFVGFIFCVIAGQLPSDDKNFIGCDCYLETGNGERVALGNMDTWTSIHSPEFFSDHVFIWYDEMCCLQNSEPKKENVEELMANYNPKVSFEFFAQSGNTWEKRESSMIRGCGVCPIYDTEYFDFIKQMELELEFTLQSIANERLVQCSDKKETLDPKQPCKKFFSHVQTGIWKSATQGLKDILL

SEQ ID NO: 19 - DNA, 3477 bp, Medicago truncatula
  source 1..3477: mol_type = genomic DNA
atggcatcatcatcttcttctcatgttcctccagtaaaatatgatgtattcataagctttagaggcacagacatacgtcatggttttctcagccatttgagaaaggaactgaggcagaagcaagttgatgcctatgtagatgatagacttgaaggaggagatgaaatatcaaaagcacttgtcaaagccattgaaggatcactcatgtctttgataatattttccaaagactatgcttcttcaaagtggtgcttggaagagcttgttaaaattgttgaatgtatggctagaaataaacaagttgtgatccctgttttctacaatgttaatccaacggatgtgaggcatcaaaaagggacttatggtgattcattagctaaacatgaaaaaaataaaggaagcttagccaaggttcgaaattggggatctgctttgactatagctgctaatctttctggatttcattcatcaaaatatggggatgaagttgagcttatagaagaaattgtcaaatgtttgtcatccaagttgaatctgatgtaccaaagtgagttaacagatcttgttggaattgaagaaaggattgcagatttggagtcattgttatgcttagattcaactgcagatgttcttgtcattggcatttggggcatgggcggtattgggaagacaactctcgcagctgcagtgtataaccgtctttgttttgaatatgaaggaagttgctttatggcaaacataacagaagaatcagagaagcatggaatgatttatctgaagaataaaattctttctatattgctaaaggaaaatgatctacatattggtacacctattggagttcccccttatgttaagagaaggttggctcgtaaaaaggttcttcttgttctggatgatatcaatgatttagagcacttagagaatttagttggaggacttgattggtttggatctggtagtagaatcatcgtaacaaccagagataagcaagtgctcggtaaaagagtcaattgtacatatgaggccaaggcattgcagtctgatgatgctattaaacttttcatcatgaatgcctttgaacatggctgtcttgacatggagtggattgagctatcaagaagggttattcattatgctaatggaaatccattagctttgaaagttttaggttcttttctttatggtaaaagcaaaatagaatgggagagtcaattgcagaaactgaagaaaatgcctcatgcaaaaattcagaatgtattgagattgagttatgatagacttgatcgagaagagaagaacatatttttgtatattgcatgtcttttgaagggatatgaagtgcaacagataatagctctgcttgatgcctgtggtttctcaacaattatcggattaagagtccttaaagataaagcccttataattgaagccaagggctctggaagatcaattgtatccatgcatgatttgatacaagaaatgggttgggagattgttcgagaagaatgtgttgaagaccctgggaaacgcagcaggttatgggatcctaatgatgttcatcaagtattgacaaataacacgggaactaaagccattaaaagcataactctgaacgtctcaaaatttgatgagctgcacttaagtcctcaagtctttggtaggatgcagcagttaaaatttcttaaatttacccaacattatggcgatgagaaaatcctgtatcttcctcagggtcttgaatctttaccaaatgacctactgctttttcaatgggttagttaccctttgaaatccttgcctcagtctttttgtgcagaaaatcttgttgagctaaaattgacttggagccgagtggaaaaactttgggatgggatccagaatattcagcatttaaagaaaattgacctcagttattccaaatatctgctagacctccctgatttttcaaaggcctcaaatcttgaagaaatagaactctttggttgcaaaagtttgcttaatgttcatccttcaattttacgtctcaacaagcttgtgcgactgaacctattttactgcaaagcgcttaccagtcttcgaagtgatacccatttacgatcccttcgcgatctcttcctcagtggttgctcaagactcgaagatttttcagtgacatctgataatatgaaagacttagctttatcttcaacagctatcaatgaattgccctcatcaattggtagtctcaaaaatctcgaaacattgactcttgatttctgcaagagcctaaacaagcttccaaacgaagttatcgacctgagatcacttcgagctctttatgttcatggatgcacacaacttgatgcgtcaaatcttcatatcctacttagcggtttagcttctttagaaacattaaagctggaagaatgtcgtaacttatccgaaattccagacaacatcagcttgctatcatcgttgcgggagttgttactaaaagaaactgatattgagagatttcctgcaagcatcaagcatctttctaagctggaaaagcttgacgtaaagggctgcaggaggcttcaaaatatgccagagcttccgccatcactaaaagagttatatgctactgattgttcgtctttagagactgtaatgttcaattggaatgcttctgatctactacaactacaagcatataagttacacacacaattccagaactgtgttaacttggatgaactttcactcagggctattgaagtaaatgctcaagtaaacatgaagaaattggcatataatcatctctcaacattaggaagcaaatttcttgatggtccagttgatgtgatctacccaggaagtaaagttccagagtggttgatgtacagaacaacagaggcttcagtgactgttgatttctcttctgctccaaaatctaagtttgttggtttcatcttctgcgtcgttgctggtcagctaccatcagacgataaaaatttcattggatgtgattgttacttggaaactggtaatggcgaaaaggtctcccttggaagcatggatacatggactagcatacattcctctgagtttttttctgaccatatttttatgtggtatgatgaattgtgttgcctgcaaaacagtaaacctgaaaaggaaaacatggatgaactcatggccagttatattccaaaggtttcatttgagttctttgctcaaagtggaaatacttggaaaaagagggagaataatatgataagagggtgtggagtctgtccaatatatgatacagaatattttgatttcattaaacaaatggaattagagttggaaatgacattgcaatctatagcaaatgaaaggtcagctcagtgcaatgataagaaagaaaaactgggtccaaaacaaccatgcaagaaattctttccaccctttcaaactggaatttggaagagtgcaacacagggcttgaaagatatattgttcttgtga

SEQ ID NO: 20 - protein, 1158 aa, Medicago truncatula
MASSSSSHVPPVKYDVFISFRGTDIRHGFLSHLRKELRQKQVDAYVDDRLEGGDEISKALVKAIEGSLMSLIIFSKDYASSKWCLEELVKIVECMARNKQVVIPVFYNVNPTDVRHQKGTYGDSLAKHEKNKGSLAKVRNWGSALTIAANLSGFHSSKYGDEVELIEEIVKCLSSKLNLMYQSELTDLVGIEERIADLESLLCLDSTADVLVIGIWGMGGIGKTTLAAAVYNRLCFEYEGSCFMANITEESEKHGMIYLKNKILSILLKENDLHIGTPIGVPPYVKRRLARKKVLLVLDDINDLEHLENLVGGLDWFGSGSRIIVTTRDKQVLGKRVNCTYEAKALQSDDAIKLFIMNAFEHGCLDMEWIELSRRVIHYANGNPLALKVLGSFLYGKSKIEWESQLQKLKKMPHAKIQNVLRLSYDRLDREEKNIFLYIACLLKGYEVQQIIALLDACGFSTIIGLRVLKDKALIIEAKGSGRSIVSMHDLIQEMGWEIVREECVEDPGKRSRLWDPNDVHQVLTNNTGTKAIKSITLNVSKFDELHLSPQVFGRMQQLKFLKFTQHYGDEKILYLPQGLESLPNDLLLFQWVSYPLKSLPQSFCAENLVELKLTWSRVEKLWDGIQNIQHLKKIDLSYSKYLLDLPDFSKASNLEEIELFGCKSLLNVHPSILRLNKLVRLNLFYCKALTSLRSDTHLRSLRDLFLSGCSRLEDFSVTSDNMKDLALSSTAINELPSSIGSLKNLETLTLDFCKSLNKLPNEVIDLRSLRALYVHGCTQLDASNLHILLSGLASLETLKLEECRNLSEIPDNISLLSSLRELLLKETDIERFPASIKHLSKLEKLDVKGCRRLQNMPELPPSLKELYATDCSSLETVMFNWNASDLLQLQAYKLHTQFQNCVNLDELSLRAIEVNAQVNMKKLAYNHLSTLGSKFLDGPVDVIYPGSKVPEWLMYRTTEASVTVDFSSAPKSKFVGFIFCVVAGQLPSDDKNFIGCDCYLETGNGEKVSLGSMDTWTSIHSSEFFSDHIFMWYDELCCLQNSKPEKENMDELMASYIPKVSFEFFAQSGNTWKKRENNMIRGCGVCPIYDTEYFDFIKQMELELEMTLQSIANERSAQCNDKKEKLGPKQPCKKFFPPFQTGIWKSATQGLKDILFL

SEQ ID NO: 21 - DNA, 3477 bp, Lotus japonicus
  source 1..3477: mol_type = genomic DNA
atggcatcttcatcttcatcttcatcttcttcttcccatgttcctagaatcaaatatgatgtctttgttagcttcagaggcacagacatacgctatggttttctcagccatttgaagaaggagcttcgccagaagcaagtggatgcctttgtagatgacaggcttgaagggggagatgaaatatcacatgcacttgtcagagcaattgaagggtcactcatgtctttggtcatattctccaaggactatgcttcttctaaatggtgcttggaggagcttgtgaaaatagttgagtgcatggacacaagtaaacaggttgtgattcctgttttctacaatgttgatccatctcatgtgcggcaccagaaaggagcttatggagatgccttggataaacatgaaaagagtaaaagaaacttagccaaggtgcaaaattggagatctgctttgagtgtagctgctaatctttctggatttcattcatcaaaatttggggatgaagttgagcttatagaagcaattgtcaaatctttgtcatccaagttgaatcttatgtaccaaagtgagttaacagaccttgttggaattgaagaacggattgcagagttggagtcacaattgcgcctagggtcgacaatggatgttcttgcccttggaatttggggcatggggggaattggtaagacaactattgcagctgcagtatataaccgtctttgttttgaatttgaagggtgttgctttatggcaaacataagggaagaatcagagaaacatggaatgatttatctgaagaataaaattctctctatattactaaaggaaaatgatctacatattggcacacctaatggagttcccccttatgttaagagaaggctcgttcgtaaaaaggttcttcttgttcttgatgatatcagtgattcagagcacctagaaattttagttggagcacttgattggtttggatctggtagcagaattattgtaacaactagagataagcaagtgcttggtaaaatagttgacagtatatatgaggcaaaggcattaaactctgatgaagctattaagcttttcattatgaatgccttcgagcaacaaagttgtgttgatatggagtggaatgagttatcaagaagggtgattcagtatgctaatggaaacccattagctttgaaagttttgggctcttttctttatggtaaaagcaagatagaatggttaagccaattgcagaaacttaagaaaatgcctcactccaaaatccaaaatgtgttgagattgacttatgatagacttgatcgagaggagaaaaacatctttctatacattgcatgttttttgaaaggatatgaattacaccgagtaatagttctgcttgatgcttgtggtttgtcaacaattattgggttaagagtccttaaagataaagcccttatcattgaagccaagggctctggaagatcaattgtgtggatgcatgacttgatacaagaaatgggttgggagattgttcgagaagaatgtattgaagaccctgggaaacgcagtaggttatgggatcctaatgatattcatcaagtgttggaaaataacacgggaactaaagccattaaaagcataactctgaacgtctcaaaaattgatgagctgtgcttaagtcctcaagtctttgctgggatgccaagattgaagtttcttaattttacccaaccttatgctgatgatcaaatcctgtattttcctcagggccttgaatcttttccaactaagctacgacttctaaattgggttagttatcctttgaaatccttgcctcagtttttttgtgcagaaaatcttgttgagctcaaaatgacttggagccgagcggaaaaactctgggatgggattcagaatcttgaacatttaaagaaaattgacctgagttattccaagtatctgatagagctcccagatttttcaaaggcctcaaatcttgaagaagtagaactctacgcttgcagaaatttgctgagtgttcatccctctatcttatctcttaacaagcttgtgcggttgaaccttttttactgcaaagcgcttaccagtcttcgaagcgaaacccatttaagatccctccgtgacctcttcctcggtggctgctcaaaactccaagagttttcagtgacttcagagagcatgaaagacttgactttaacttcaactgctatcaatgaattgccctcatcaattgggagtctcagaaacctggaaatgctgactcttgataactgcaaaagtctaagcaatctttcaaacaaggttgctgagctaagatcacttcgggagcttcatgttcatggttgcacacagcttgatgcatcaaatctgcatgtcctggttaatggtttgaggtctttggaaacattaaagctgcaagaatgtcgaaacttgttcgagattccagacaacatcagcctcctatcttcattgcgccaattgttactaaccggaacagatattgagagatttcctgcaacaataaagcaactttctaaccttgaaaagattgacttaagggattgcaagaggctttgttatctgccagaacttccactatccttgaaagagttacatgccaataactgttcatccttggagactgtaatgcttacttcaagagctattgaactactacatcaacaagcaaataagatgcacacacaattccaaaattgtgtaaatttggataagtattcactcagtgctattggagttaatgctcatgtaagcatgaagaaattggcatatgataatctctcatcattaggaagcaagtttcttgatggtccagttgattttatgtaccctggaaaaaaagttccagagtggttcatgtacaggtcaacacaggcttcagtaactcttgatctgtgttctgctccgcgttccaaatttatgggtttcatattctgcgtcattgtcggtaagttcccatcagatgacaacaacttcatagggtgtgactgttacctggaaaccggtaacagcgaaagggtcaaaatggatgcatggactagtatccacgccggtaaatttgtttctgatcatgtctgtatgtggtatgatcaacgatgctgtctgcaaagcagtgaatgtgaaaatgatagtatggaaatggaagaacatgtggctggttgcaatattccaaaggtttcatttgaattctttgctcaaagtggaagcacttggaaaaagcatgatgatatcattgtaaaagggtgtggagtgtgtcctttatacgacacagaatatgacaatttcataaagcaaatggaattggagttggaaaccactttgcagtctatagcaaaaatttcagttgcatgctctgttaagaaagaaacacaatgtaagaaattctttcccccccttcagattgggacatggaagagtgcaacacagggattgaaagacatattgttcttctga

SEQ ID NO: 22 - protein, 1158 aa, Lotus japonicus
MASSSSSSSSSSHVPRIKYDVFVSFRGTDIRYGFLSHLKKELRQKQVDAFVDDRLEGGDEISHALVRAIEGSLMSLVIFSKDYASSKWCLEELVKIVECMDTSKQVVIPVFYNVDPSHVRHQKGAYGDALDKHEKSKRNLAKVQNWRSALSVAANLSGFHSSKFGDEVELIEAIVKSLSSKLNLMYQSELTDLVGIEERIAELESQLRLGSTMDVLALGIWGMGGIGKTTIAAAVYNRLCFEFEGCCFMANIREESEKHGMIYLKNKILSILLKENDLHIGTPNGVPPYVKRRLVRKKVLLVLDDISDSEHLEILVGALDWFGSGSRIIVTTRDKQVLGKIVDSIYEAKALNSDEAIKLFIMNAFEQQSCVDMEWNELSRRVIQYANGNPLALKVLGSFLYGKSKIEWLSQLQKLKKMPHSKIQNVLRLTYDRLDREEKNIFLYIACFLKGYELHRVIVLLDACGLSTIIGLRVLKDKALIIEAKGSGRSIVWMHDLIQEMGWEIVREECIEDPGKRSRLWDPNDIHQVLENNTGTKAIKSITLNVSKIDELCLSPQVFAGMPRLKFLNFTQPYADDQILYFPQGLESFPTKLRLLNWVSYPLKSLPQFFCAENLVELKMTWSRAEKLWDGIQNLEHLKKIDLSYSKYLIELPDFSKASNLEEVELYACRNLLSVHPSILSLNKLVRLNLFYCKALTSLRSETHLRSLRDLFLGGCSKLQEFSVTSESMKDLTLTSTAINELPSSIGSLRNLEMLTLDNCKSLSNLSNKVAELRSLRELHVHGCTQLDASNLHVLVNGLRSLETLKLQECRNLFEIPDNISLLSSLRQLLLTGTDIERFPATIKQLSNLEKIDLRDCKRLCYLPELPLSLKELHANNCSSLETVMLTSRAIELLHQQANKMHTQFQNCVNLDKYSLSAIGVNAHVSMKKLAYDNLSSLGSKFLDGPVDFMYPGKKVPEWFMYRSTQASVTLDLCSAPRSKFMGFIFCVIVGKFPSDDNNFIGCDCYLETGNSERVKMDAWTSIHAGKFVSDHVCMWYDQRCCLQSSECENDSMEMEEHVAGCNIPKVSFEFFAQSGSTWKKHDDIIVKGCGVCPLYDTEYDNFIKQMELELETTLQSIAKISVACSVKKETQCKKFFPPLQIGTWKSATQGLKDILFF

SEQ ID NO: 23 - DNA, 3429 bp, Prosopis alba
  source 1..3429: mol_type = genomic DNA
atggcatcttcttctcagttttctggtgcaaagtatgacgtatttatcagctttagaggctcagacatacgccatggttttcttagccatttgagaaaggaattgcggcagaaacaaattgaagcccatgtggatgacagactcgaaagagaagataaaatgtcacctgcacttggaagagcgattagagaatcacttatctcactaatcatattttcaaaggattatgcttcatcaaagcggtgcttagacgagctggtgcagattattgagtgcagggacaaccagaaccagattgtaattcctgttttctataacacagatccctcagacgtgaggcatcaaaaaggttcttatggagacgctttagccaaacatgatgagtattacaaggaaaaggtgccaatttggagatcatctctgaataaaactgctaacttatctgggtttcactatttatccaaccatgcggatgaggctgagcttatagaagatattgccaaatgtttgtcaacaaggttgaatcttatgtaccaaagtgactttacagatcttgttggaattcatgaaagaattgcagatttagaatcactaatgaaccaaggatcagaagatgttcgtgtcattggaatttggggtatgggcggtatcggtaagacaactattgccgctgtaatatttaaccgattttgttccgaatttgaaggacattgttttttggccaatgtgagagaagaatcaaagaatcatggaatcatttgtttgaagaataaaatgttctctaaactactcaaggaaaatgatttacatatcggtacaccaaatggaatacccccttgtgtcaagagaaggcttcttcgaaaaagggttcttgttgttcttgatgacattagtgattcggatcaactagataatttagtgggagcacttaactgctttggacttgggagtagaatcattataacaactagagataagcaagtgctagctaaaaatgttgataaaatatacgaagttaagcctttgattcgtgatgatgctgttcgactttttatgttgaatgcctttgagaataatgatgtggaaatggggtggattgaattgtcaaggaagtttattagttatgctaaaggcattccgttggctctgaaagtcttaggttccttcctttatggtaaaagcaggcaagaatgggaaagccaacttgagaagctcagcaaaatgccaaatgcaaaaattcaagatgtgatgagactgggttatgatgatctagatagagaagagaagaacattttcttatatattgcatgttttctcaaaggatatgaatcacaacagataattgttttgcttaattcttgtggtttctcaaccattattgggttaagggtccttcgagacaaagctcttataactgaagtatcaaactccagaaaatcaataatatctatgcatgatttgatccaagagatgggtcgggaaattgttggtgaagaatctattgacaatcctggaaaacgtagtcgattatgggatgctaatgatatttatcaagtattagcacataacacgggcactaaagccattcaaagcattactctgaatgtagcaaaaattggagggctctgcttaattcctcaagtcttttctaagatgacagagctaaagtttctcaagttttcccaacatgattttcatgaacaaatcctccaccttcctcaaggacttgaatctttgccaaatgaactgagattttttgagtgggtttgctatcctttaccatccttgccttcctcattctgtcctaaaagtcttgttgaacttaaaatgacttggagtcgagtggtaaagctctgggatggggttttggattttgtgaatttaaagaaaattgacctcagttactccacgcatctgttggaactcccagatttttcgatggcgaaaaaccttgaagaagtagaactctccagctgcaagagcttgcgcaaggttcatccctctatcctaaccatccacaaacttgtgaaactgaacctatcatactgtaaatcacttaccatacttagaggtgatgtccatttaagatccctcagtaatctttctcttagtgaatgctcaagactcaggcaattctcagtgacatcagagaatatgaagaacttaagtttgacaggcacatctatcaatgaattgccttcgtctgtgaggcttttaaacaaacttgaaacattatacttagatcactgtaagaaccttaagaatcttccaaacaagttttctaaactcagttctcttagagaacttcggatcattggttgcatccagcttgatgcatcgaatctacacttgttactcgaaggctcaaggttcctagaaacattgttgttgcaagattgtcgtaatttatttgaacttccagacaatattagattcttatcctcattgcagcatctatcactacatgaatcagatattgtgaagttgcctgcaagcatcaagcatctccaacaacttgaaaagcttaacttgggtaattgcaggaagcttcattctgtgcccgagcttccaccatccattaaagaactatataccactaattgcacttccctgaagagagtgatgatttcttccatggctgctgatcaattgagagaaaaaatacgagtgggttctagatttcagaattgtgtaaacttggatgaacattcacttagagctgtagcaatgcattctcatgtcaacatgaagagattggcatatgagcatttatcaaatctaggacaaggtaacatcctaggtggtccaattgatgttatttatccaggaagcaatgttccagagtggttcgtgaacaggacaaggcagtcttcagtaactgtggacttgtcttctgctccgccgtcttccgtgtctctgggtttcatcttttgtgtggttgtttctaggtttccatcaaacgacaagaactttgttgggtgtgattgctacttggaaataaatggtgaaagggacatgaacatgggtgcatggacgagcataaatacttgtgaatttatgtctgatcatgtatgtatatggtacgatgaacgatgttgtctgaaaacaagtgaaagcatggaagctgatgaaagcaataatctgaagatatggttcgagttctttgctcagactgggaatacctgggagaagagaagggatattgcggttaaagagtgcggagtttgccccatttatgactcagaatatcacaccttcattgaagaactggagttgaggccgcaactgggatggtttgatgagaatgaagatgaggaagaagcaatccattttgcacagaaatccaatcaacacatgtgtccacctcttccaactaagactaactggaagacatcgacacagggcttgaaagatctcatcaacctatga

SEQ ID NO: 24 - protein, 1142 aa, Prosopis alba
MASSSQFSGAKYDVFISFRGSDIRHGFLSHLRKELRQKQIEAHVDDRLEREDKMSPALGRAIRESLISLIIFSKDYASSKRCLDELVQIIECRDNQNQIVIPVFYNTDPSDVRHQKGSYGDALAKHDEYYKEKVPIWRSSLNKTANLSGFHYLSNHADEAELIEDIAKCLSTRLNLMYQSDFTDLVGIHERIADLESLMNQGSEDVRVIGIWGMGGIGKTTIAAVIFNRFCSEFEGHCFLANVREESKNHGIICLKNKMFSKLLKENDLHIGTPNGIPPCVKRRLLRKRVLVVLDDISDSDQLDNLVGALNCFGLGSRIIITTRDKQVLAKNVDKIYEVKPLIRDDAVRLFMLNAFENNDVEMGWIELSRKFISYAKGIPLALKVLGSFLYGKSRQEWESQLEKLSKMPNAKIQDVMRLGYDDLDREEKNIFLYIACFLKGYESQQIIVLLNSCGFSTIIGLRVLRDKALITEVSNSRKSIISMHDLIQEMGREIVGEESIDNPGKRSRLWDANDIYQVLAHNTGTKAIQSITLNVAKIGGLCLIPQVFSKMTELKFLKFSQHDFHEQILHLPQGLESLPNELRFFEWVCYPLPSLPSSFCPKSLVELKMTWSRVVKLWDGVLDFVNLKKIDLSYSTHLLELPDFSMAKNLEEVELSSCKSLRKVHPSILTIHKLVKLNLSYCKSLTILRGDVHLRSLSNLSLSECSRLRQFSVTSENMKNLSLTGTSINELPSSVRLLNKLETLYLDHCKNLKNLPNKFSKLSSLRELRIIGCIQLDASNLHLLLEGSRFLETLLLQDCRNLFELPDNIRFLSSLQHLSLHESDIVKLPASIKHLQQLEKLNLGNCRKLHSVPELPPSIKELYTTNCTSLKRVMISSMAADQLREKIRVGSRFQNCVNLDEHSLRAVAMHSHVNMKRLAYEHLSNLGQGNILGGPIDVIYPGSNVPEWFVNRTRQSSVTVDLSSAPPSSVSLGFIFCVVVSRFPSNDKNFVGCDCYLEINGERDMNMGAWTSINTCEFMSDHVCIWYDERCCLKTSESMEADESNNLKIWFEFFAQTGNTWEKRRDIAVKECGVCPIYDSEYHTFIEELELRPQLGWFDENEDEEEAIHFAQKSNQHMCPPLPTKTNWKTSTQGLKDLINL

SEQ ID NO: 25 - DNA, 3453 bp, Prosopis alba
  source 1..3453: mol_type = genomic DNA
atggcgtattcttctcagtgttctgggataaaatatgatgtctttatcagcttcagaggcacagacactcgccatggttttcttagtcatttgaggaaggaattgcggcagaagcaaattgatgcctatgtggacgacagacttgaaagtggagatccaatatcaccggcacttttaaacgccatcaaagaatcactcatgtcattgatcatattttcgaaggattatgcttcttcaaggtggtgcttagaagagctggtgcagattattgagtgtatggagaagcaaaaacagattgtaattcccgttttctataacacagatccctcagatgtgaggcatcaaaaaggatcttatggagatgcttttgtcaaacatgacaaaaattacaaggaaaaggtgccaatttggaaatcagctttgaataaagcggctaacttgtctgggtttcattcatcaagttacgaggacgaggctgagcttatagaaaaaattgccaagcgtttgtcgacaaggttgaatattatgttccaaagtgacttaacaggcctcgttggaattgatgaaaggattgcagatttagaatcactaatgaaccaaggctcaaaagatgttcgagtcattggaatttggggtatgggcggtatgggtaagacaactatcgcaactgcagtatttaatcgattctgttctgaatatgaaggatattattttttggcaaatgtgagagaagaatcagagaatcatgggattatctatttgaagaataaaattctttctatactgctcaaggaaaatgatttgcatattggtacaccaaatggaataccaccttatgttaagagaaggcttcttcgaaaaagagttcttgttgttcttgatgatattagtgattctgatcaactggaaaatttagtgggagcacttgattgctttggatcgggtagcagaatcattataacaacaagggataggcaagttctagctaaaggagttgatgaaatatacgaagttaagcctttgatatttgatgatgctgctcaactttttatgtcaaatgcctttgggaataattatcttgacatggagtggattgaattatcaaggaaggtgatcagttatgctaaaggcgttccattggctttgaaggttttgggttcctttctttatggcaaaagcaaggaagaatgggagagccaacttgagaagctcaacaaaatgccacatgcaaagattcaggatgtgatgagattgagttatgataaacttgatagggaagagaagaatattttcttgtatattgcttgtttcttcaaaggatataaatcacggcaagtaatactttttctaaatgcttgtggtttttcaacaattattgggttaaaagtccttgaagacaaagctcttatcactgaaatcaaaggatccaacgcaggagcatcaaaaatctttatgcatgatttgatagaggaaatgggttgggagattgttcgtgaagaatcaattcatgatcctggaaaacacagtcggttatgggatgctaatgatatgtgtcgagtattggaacataatctggggactaaagccattcaaagcataacttcgaatgtgacaaaaattgaagagctctgcttaactccccaggtctttgccgagatgtctgagctaaaacttcttagttttccccagaatgatgtcaatggacaaatcctatgccttccttgtggccttgaatctttgccgtatgaactgcgatttcttcattgcggttgctgtcctttaaaatccttaccttccacattccgtccagaaagtctcgttgaacttaacatgacttggaatcgtgtggaaaaactttgggatggagttctgcgtcttgtgaatttgaagaagattgacctcagtaactccaagcatctgttggaactcccagatttttctatggccagaaaccttcaagaagttgtactctacaactgtaaaagcttgcgtaaggttcatccttctattttaaccatccacaagcttgtgaatttgaacctatcgtattgcaaatcacttaccagccttagaagtgatgtacatttgagatccctgagtagtctttctcttggtgaatgctcaaggctcagggaattctcagtgacatcagagaatatgaggaacttgagtttggcaggcacagctatcaatgaattgccttcatccatcaggcttctaaacaaacttgaaacattcgacttacatcgttgcaaaaagcttaagaatcttccaaacaagtcagctaacctaagttctcttcgagaactttggatatatggttgcgtgcagattgatgcctcgaatttacgcttcctacttgatggctcaagattcttagaaacattggtactgcaagattgtcataatttatttgaacttccagacaccatcagattcttatcctcattgaaacatttatccctaaatgaaactgatattaagaggttgcctgcaagcatcaagcatcttccacaacttgaaaaacttgatttgagtaattgtaggaagcttcagtctttgcctcatcttccactaaccgttaaagaactatatctcactaactgcacttccctggagagagtcatgttttcttcaatggcttctgatcatctgaagggaaaaatgagatatagcactagtttccagaattgcgtgaacttggatgcacattcactcagagctatagaagtaaattctcatgtcaacattaagagattggccattgaacatttatcaactctagaaaaaagtaagatcctaggtggtccagttgatgtcatttatccaggaagcaatgttccggagtggttcactaacaggacaaggcagtcatcagtaactattgacttatcttctgctccaccttccatgttcgtgggtttcattttttgtgtggttgtctgtaagtttccatcagatgacaagaaccttgtcggatgtgactgctacatggaaattaatggtaaaagagacaagaatatgggtgcttggactagcatatctgcttgtgaatttatgtctgatcatgtatgtctgtggtatgatgaacgatgttgtctgaaaactaatgaaagcaaggaggaagatgaaagtaatcgcaagaaaatatcatttgagttctttgcacaaactggggatatttggaagaagagcaaggatattgtgataaaagagtgtggagtctgccccatatatgcctcagaatatgataaattcattgagcaaatggagttggagttgaaatcagagcttaaagcaattgcaataggagttagtggaagctttgatgaaaagaaaattgaggagacagtcccttttgttaaaaaatccaatcgatttatctttcctccccttgcaaatggaacttggaagaaagcaacacaaggcttaaaagatctcatcaaattgtga

SEQ ID NO: 26 - protein, 1150 aa, Prosopis alba
MAYSSQCSGIKYDVFISFRGTDTRHGFLSHLRKELRQKQIDAYVDDRLESGDPISPALLNAIKESLMSLIIFSKDYASSRWCLEELVQIIECMEKQKQIVIPVFYNTDPSDVRHQKGSYGDAFVKHDKNYKEKVPIWKSALNKAANLSGFHSSSYEDEAELIEKIAKRLSTRLNIMFQSDLTGLVGIDERIADLESLMNQGSKDVRVIGIWGMGGMGKTTIATAVFNRFCSEYEGYYFLANVREESENHGIIYLKNKILSILLKENDLHIGTPNGIPPYVKRRLLRKRVLVVLDDISDSDQLENLVGALDCFGSGSRIIITTRDRQVLAKGVDEIYEVKPLIFDDAAQLFMSNAFGNNYLDMEWIELSRKVISYAKGVPLALKVLGSFLYGKSKEEWESQLEKLNKMPHAKIQDVMRLSYDKLDREEKNIFLYIACFFKGYKSRQVILFLNACGFSTIIGLKVLEDKALITEIKGSNAGASKIFMHDLIEEMGWEIVREESIHDPGKHSRLWDANDMCRVLEHNLGTKAIQSITSNVTKIEELCLTPQVFAEMSELKLLSFPQNDVNGQILCLPCGLESLPYELRFLHCGCCPLKSLPSTFRPESLVELNMTWNRVEKLWDGVLRLVNLKKIDLSNSKHLLELPDFSMARNLQEVVLYNCKSLRKVHPSILTIHKLVNLNLSYCKSLTSLRSDVHLRSLSSLSLGECSRLREFSVTSENMRNLSLAGTAINELPSSIRLLNKLETFDLHRCKKLKNLPNKSANLSSLRELWIYGCVQIDASNLRFLLDGSRFLETLVLQDCHNLFELPDTIRFLSSLKHLSLNETDIKRLPASIKHLPQLEKLDLSNCRKLQSLPHLPLTVKELYLTNCTSLERVMFSSMASDHLKGKMRYSTSFQNCVNLDAHSLRAIEVNSHVNIKRLAIEHLSTLEKSKILGGPVDVIYPGSNVPEWFTNRTRQSSVTIDLSSAPPSMFVGFIFCVVVCKFPSDDKNLVGCDCYMEINGKRDKNMGAWTSISACEFMSDHVCLWYDERCCLKTNESKEEDESNRKKISFEFFAQTGDIWKKSKDIVIKECGVCPIYASEYDKFIEQMELELKSELKAIAIGVSGSFDEKKIEETVPFVKKSNRFIFPPLANGTWKKATQGLKDLIKL

SEQ ID NO: 27 - DNA, 3429 bp, Prosopis alba
  source 1..3429: mol_type = genomic DNA
atggcgtgttcttctcggtcctctggtataaagtatgatgtctttatcagcttcagaggcacagacatacgccatggttttcttagtcatttgaggaaggaattgcagcagaaacaaatcgatgcctatgtggacaacagacttgaaagtggagatcaaatatcactggcacttgtaagagccatcaaagaatcactcatgtcattgatcatattttcagaggattatgcttcttcaaggtggtgcttagacgagctggggcagattattgagtgtatggagaagcagaaacagattgtaattcctgttttctataatacggatccctcagatgtgaggcatcaaaaggggtcttatggagatgcttttgtcaaacatgacaaacataataaggaaaaggtgccaatttggaaatcagctttgaataaagcggctaatttatctgggtttcattcatcaaattatgaggatgaggctgagcttatagaagtgattgccaagcgtttgtcagaaaagttgaatattatgttccaaagtgacttaacgggccttgttggaattgatgaaaggattgcagatttagaatcgctaatgaaccaagggtcagaaaaagtccgagtcattggaatttggggcatgggcggtatcggtaagacaactatcgcagatgcagtatttaatcgattctattctgaatatgaaggatattgttttttggcaaatgtgagagaagaatcagagaatcatgggattatgtatttgaaaaataaaattctttctatactactcaaggaaaatgatttgcgtatcggtacgcccaatggaatacctccttatgttaggagaaggcttcttcgaaaaagagttctggttgttcttgatgatataagtgattctgatcaactggaaaatttagtgggagctcttgactgctttggatctggtagtagaatcattataacaacaagggataagcaagtgctggctaaaagagttgataaaatatacgaggttaagccattgatgtttgatgatgctgttcaactctttatgtcgaatgcctttgagaatgattatctagacaaggagtggattgaactatcaaggaaggtgattagttatgcaaaaggcattccgttagttctgaaagttttgggttcttttctttatggcaaaagcaaggaagagtgggagagccaacttgagaagctcaacaaaatgccacatgctaaaattcaggatgtgatgagattaagttatgataaactagatagagaagagaagaatattttcttgtacgtcgcatgtttccttaaaggatacaaatcacaacaaataatagttttactaaatgcttgtggtttttcatcaattattgggctcaaagtgcttctagacaaagctcttatcactgaaatcaaaggatcaaattcaggaggttcaagaatatttatgcatgatttgatacaggaaatgggttgggagattgttcgtgaagaatcaatttatgatcctggaaaacgcagtcgattgtgggatgctaatgatatatatcgagtattggagcataacctggggactaaagccattcaaagcataactttgaatgtggcaaaaattgaagagctctgcttaactcctcaagtctttgctgagatgtcagagctaaaattgcttagtttttcccagaatgatgttgatgaagaaatcctacgccttcctcatggccttgaatctttgccaaatgaactgagatttcttcactgggattgctgtcctttgaaatctttaccctccacattccgtccagaaagtcttgttgaacttaacatgactaggagtcgtgtggagaaactttgggatggagccctgcgtcttgtgaatttaaagaaaattgacctcagtaattccaagcgcctattggaactcccagatttttccatggccaaaaatcttgaagaagttgaactctacaactgtaaaagcttgcgtaaggttcatccttccattttaaccatccacaaacttgtgaaattgaaccttatatattgcacatcacttaccagccttagaagtgaagtccatttaagatccctgagtagtctttctcttggtgattgctcaaggctcaaagaattctctttgacatcagagaatatgagggacttgagtttggcgggcacagctatcaatgaattgccttcatccatgaggcttctaaacaaacttgaaacatttgacttgcatcgttgtaaaaaccttaagaatcttccaagcaagtcaactaacctgcattctcttcgagaactcaggatctttggttgcatgcagcttgatgcatcgaatttacacttgttacttgatggctcaaggttcttggaaacactgttactgcaagattgtcataatttatttgaacttccagacaccatcagattcctatcctcattgaagcatttatccctaaatgaaacagatattgagaggttgcctacaagcatcaagcatcttcctcaacttgaaaaacttgacttgagtaattgtaggaggcttgagtctttgcctaatcttccactaagcattaaagaactatatgttactaactgcacttccctggagagagtcatgttttcttcaatggctgccgatcatctgacaggaaaaattcgagtctgcactagattccagaattgtgtgaacttggatgcacagtcactcgcagctataggagtgaattctcatgtcaacattaagagactggcaaatgaacatttatcaactataggaaaaagtaagatcctagatggcccagttgatgttatttatccaggaagcaatgttccagggtggttcatcaacaggacaaggcagtcatcggtaactattgacttatcttctgctccaccttccgtctttatgggtttcattttttgtgcggttgtctgtaagttcgcatcaaatgacagcaacctcgtcggatgtgactgctacatggagattaatggtacaaggaacaagaatatgggtgcatggactagcatatctgcttgtgaatttatgtctgatcatgtatgtctgtggtacgatgaacgatgctgtctgaaagctgatgaaaacatggaagcagatgaaaccaataaccagaaaatatcatttgagttctttgcacaaactgggagtatttgggagaagaaaaaagatattgtgataaaagaatgtggagtctgccccgtatatgcctcagaacatcacaaattcattgagcaaatggagttggagttgaagtcacagcttaaagcaattggtgagaacaaacatgagaagacaatcccttcagctcaaaagtccaatgaacatatcttttcaccacttcccagtggatcttggaagaaagcaacacaaggcttaagagatctcctcaacttgtga

SEQ ID NO: 28 - protein, 1142 aa, Prosopis alba
MACSSRSSGIKYDVFISFRGTDIRHGFLSHLRKELQQKQIDAYVDNRLESGDQISLALVRAIKESLMSLIIFSEDYASSRWCLDELGQIIECMEKQKQIVIPVFYNTDPSDVRHQKGSYGDAFVKHDKHNKEKVPIWKSALNKAANLSGFHSSNYEDEAELIEVIAKRLSEKLNIMFQSDLTGLVGIDERIADLESLMNQGSEKVRVIGIWGMGGIGKTTIADAVFNRFYSEYEGYCFLANVREESENHGIMYLKNKILSILLKENDLRIGTPNGIPPYVRRRLLRKRVLVVLDDISDSDQLENLVGALDCFGSGSRIIITTRDKQVLAKRVDKIYEVKPLMFDDAVQLFMSNAFENDYLDKEWIELSRKVISYAKGIPLVLKVLGSFLYGKSKEEWESQLEKLNKMPHAKIQDVMRLSYDKLDREEKNIFLYVACFLKGYKSQQIIVLLNACGFSSIIGLKVLLDKALITEIKGSNSGGSRIFMHDLIQEMGWEIVREESIYDPGKRSRLWDANDIYRVLEHNLGTKAIQSITLNVAKIEELCLTPQVFAEMSELKLLSFSQNDVDEEILRLPHGLESLPNELRFLHWDCCPLKSLPSTFRPESLVELNMTRSRVEKLWDGALRLVNLKKIDLSNSKRLLELPDFSMAKNLEEVELYNCKSLRKVHPSILTIHKLVKLNLIYCTSLTSLRSEVHLRSLSSLSLGDCSRLKEFSLTSENMRDLSLAGTAINELPSSMRLLNKLETFDLHRCKNLKNLPSKSTNLHSLRELRIFGCMQLDASNLHLLLDGSRFLETLLLQDCHNLFELPDTIRFLSSLKHLSLNETDIERLPTSIKHLPQLEKLDLSNCRRLESLPNLPLSIKELYVTNCTSLERVMFSSMAADHLTGKIRVCTRFQNCVNLDAQSLAAIGVNSHVNIKRLANEHLSTIGKSKILDGPVDVIYPGSNVPGWFINRTRQSSVTIDLSSAPPSVFMGFIFCAVVCKFASNDSNLVGCDCYMEINGTRNKNMGAWTSISACEFMSDHVCLWYDERCCLKADENMEADETNNQKISFEFFAQTGSIWEKKKDIVIKECGVCPVYASEHHKFIEQMELELKSQLKAIGENKHEKTIPSAQKSNEHIFSPLPSGSWKKATQGLRDLLNL

SEQ ID NO: 29 - DNA, 3309 bp, Prosopis alba
  source 1..3309: mol_type = genomic DNA
atggcatcatcatcatctcagttctctcgaataaaacatgatgcctttatcagcttttgcggcacagatacacgttctggttttcttagtcatttgaaaaaagaattgcggcataagcaaattgatgccttcgtgtatgataataagcttgagagaggagatctaatatcaccagaactttctgtagcaatagaagaatcattcatttcgcttatcatattttcaaagagctatgcttcttcgacatggtgcttgaaacaactggtgaaaatcatggagtgcaaggacaaatatcaacagattgtgattcctgttttctacgacattgatccaacagatgtaaggcaccaaaaggggtattatgcagatgctttagctcagcatgactcaagttacaaggaagaggtgccaaattggaaatatgccttgaataaagctgctaatttatctggatttcattcatcaaagtatggggatgaagctaagcttattgaagaaattgccaaatccatgtcaaacaagttgaataatgtgtatcaaagtgacttcacagaccttgttggcattgatgaaagaattgcagatttagaatctctaatggatcaagggttagaagatgtccgtgtcattggaatttggggcatgggcgggattggcaagacaactattgctgcagcagtttttaatcgattttgttctgaatatgaaggttgttgtttttcggcaaatgtaagggaagaatcaaagaaaggtgggattatccatgtgaagaacaaaattctttctatactactgaaggaaaatgatttatacattggcactccctatggtgtacccccttatgtgaagagaaggcttctccggaaaaaggttcttgttgttcttgatgatgttaatgatttgggtcaactagaaaatttagttggagcatttgattgttttggaccaggtagtagaatcattattacaactagagataaggaagtacttggtcaaaaggttgacaaaatttatgaggtgaagccactggattatgaaaaagcactccaacttttcaacatgaatgcctttaaacaaagctttctagacatggaatggagtgagctatcaaagaggatggttgcgtatgctaagggacttccattggctcttaaagttttgggttcttttctctatgggaaaagtaaagaagaatggaaaagccagttgaaaaagttggagaaaaagccattcaaaaaaatccatgatttgttgagattgagctacaatagtctagaccgccaacagaaagagatctttttgtatatcgcatgtcttttcaaaggatatcaatatcgtgaaataatagctcttctagatgcttgtggttttacaacaactattgagttaaaagtgcttcgagacaaagctcttataactgaagtcattaatttagtatctaaaaagcccatggtatctatgcatgatttgatacaggaagtgggctgggaggttgttcgtgaagaatcaataaaagatcctggaaaacgcagtcgtttatgggatcctgatgatatttatgaagtactgataaataatatggggactgaaacgattcaaagcataagtctggacatgacaacaactggagatctgtccttaaatcctcaagtctttgctaggatgcacaagatgaagtttcttaaaatatttactcagatatcttccttggatgcacgtgttcatctacctcaaggccttgaatttttgcccaatgaactaagaattcttctttggcagaattaccctttgaaattcctgcctccaacattttctgcagaaaatcttgttcaacttgttatgaactacagcaatgtggaaaaactttgggatggagttgtgcctcttgtgaatttaaagaaatttgacctcaggcactctacgaatctgctagctctcccagatttttctacggccaataatcttgaatcggtctcactgttcagttgtaagaacttgcctcttgttcatccatctattttatctctccaaaagcttgagtccttggatctatcatactgcgaaaagcttaccagtcttgtgagtgatatacctttgaaatccctccgatcactccgtctagcttgctgctcaagactgaagaaattctcattgacatcagagaacatgagagagttagatttatcagagacagctattgaggaattgccattgtccttaggatgtctcagccagcttgaaacattatctctatggcgttgtgaaagtctcaagcatcttccagataacttggttggcttgagatctctgcgttatctcaaggtaactggttgtatgaaacttcaatctctaccagagcttccaccatccattgaaacattatgggcttcagagtgttcttccttggcgaccgtaccagtatcttctgatccactcaacacaaacatgaaggaaattcgtctggcaaattgcatgaacttggatgaaaatttgctcaatgatattggatggaacattcatctcaagctgatgaatcattatgcatttgaacgggtatcctcagaagaccaggaggagcaaattgatgtttatgttcgaactggaggaatataccctggaagcaaagttccaaactggttcaagtatagaacatctcaggcttcgataactattggtgtgaaattcaatattcctgatgccttcattttctgcgtggttctttctcagttcccattaagcagtttccttatctccatcgcagttgactgcttcctggaaaacaatgatggtgagaagcttcatgttaagtctttggacctcgtgttgcctggcacagagcttaattcagatcatgtgtgtttattttacgtcgaatgtttgggttttataactggaaggattgaagaaagtagagactgttatgagcacaaagtttcattcgaattctcggctcagtcagaggttgagcaagaagaggaggataaagagcaagaagaggaggataaagagattgtgataaaagggtgtggggtttacccgatatatgcctcgcaacttaatttcataggggatgttaacaggttaaaacccagaaaagttggagttcagagctgtgatgagctccggccacttcctccgttgttggaagccggtgaagccattgatgatcctgaaagttctggtgagaagaaagatgaggaagagagagtctttttagctcaaaaatccagcggatatatattttcaccacttccaactggaacatggaagaaaccaactcaaggcttgaaagatcttgtcaacctatga

SEQ ID NO: 30 - protein, 1102 aa, Prosopis alba
MASSSSQFSRIKHDAFISFCGTDTRSGFLSHLKKELRHKQIDAFVYDNKLERGDLISPELSVAIEESFISLIIFSKSYASSTWCLKQLVKIMECKDKYQQIVIPVFYDIDPTDVRHQKGYYADALAQHDSSYKEEVPNWKYALNKAANLSGFHSSKYGDEAKLIEEIAKSMSNKLNNVYQSDFTDLVGIDERIADLESLMDQGLEDVRVIGIWGMGGIGKTTIAAAVFNRFCSEYEGCCFSANVREESKKGGIIHVKNKILSILLKENDLYIGTPYGVPPYVKRRLLRKKVLVVLDDVNDLGQLENLVGAFDCFGPGSRIIITTRDKEVLGQKVDKIYEVKPLDYEKALQLFNMNAFKQSFLDMEWSELSKRMVAYAKGLPLALKVLGSFLYGKSKEEWKSQLKKLEKKPFKKIHDLLRLSYNSLDRQQKEIFLYIACLFKGYQYREIIALLDACGFTTTIELKVLRDKALITEVINLVSKKPMVSMHDLIQEVGWEVVREESIKDPGKRSRLWDPDDIYEVLINNMGTETIQSISLDMTTTGDLSLNPQVFARMHKMKFLKIFTQISSLDARVHLPQGLEFLPNELRILLWQNYPLKFLPPTFSAENLVQLVMNYSNVEKLWDGVVPLVNLKKFDLRHSTNLLALPDFSTANNLESVSLFSCKNLPLVHPSILSLQKLESLDLSYCEKLTSLVSDIPLKSLRSLRLACCSRLKKFSLTSENMRELDLSETAIEELPLSLGCLSQLETLSLWRCESLKHLPDNLVGLRSLRYLKVTGCMKLQSLPELPPSIETLWASECSSLATVPVSSDPLNTNMKEIRLANCMNLDENLLNDIGWNIHLKLMNHYAFERVSSEDQEEQIDVYVRTGGIYPGSKVPNWFKYRTSQASITIGVKFNIPDAFIFCVVLSQFPLSSFLISIAVDCFLENNDGEKLHVKSLDLVLPGTELNSDHVCLFYVECLGFITGRIEESRDCYEHKVSFEFSAQSEVEQEEEDKEQEEEDKEIVIKGCGVYPIYASQLNFIGDVNRLKPRKVGVQSCDELRPLPPLLEAGEAIDDPESSGEKKDEEERVFLAQKSSGYIFSPLPTGTWKKPTQGLKDLVNL

SEQ ID NO: 31 - DNA, 3432 bp, Prosopis alba
  source 1..3432: mol_type = genomic DNA
atggactctttttccaagttgtcgggtatacaacatgatgtcttcatcagcttcaggggcacagacacacgtcatggtttccttagccatttgagaaaggaactgcggcagaagcaaattgatgcctacgtagatgacagacctgaaagtggagatcaaatatcaccggcgcttttaagggccattaaagaatcactcatttcattgatcatattttcaaaggactatgcttcttcaaagtggtgcttagaagaacttgtgcagattattgaatgtatggagaagcaaaaacagattgtaattcccgttttctataacacagacccctcaaatgtgagacatcagaaaggatcttatggagatgctttatcgaaacatggcgaatattatgaggaaagggtgccaatttggagatcagctttgaaagaggctgctaatttatctgggtttcattcatcaaattatgaggatgaggctgagcttatagaagaaattgccaaatctttgtcaacaaagttgaatattttgtaccgaggtgacttaacagatcttgttggaattgatgaaagaattgcagaattagaatcactgatggatgaagggtcaaaagatgtccgtgtcattggaatttggggtatgggcggtatcggtaagacaaccatcgcagctgcaatattctatcgatcttgttttgaatacgaagggcattgttttttggcaaacgtgagagaagaagctgaaaatcatggcattatttatttgaagaataaaatgctttcttcactactgaaggaaaatgatttacaaattggcaccccatatctcatgccccctttcgttaggagaaggcttcttcgaaaaaggattctggttgttcttgatgatattagtgattcagatcaccttgaaaatttagtaggagcacttgattgctttggatctggtagtagagtcttaataactactagagataaacaagtacttgctaaaagagctgatgaaatatacgaggttaagccattgatgtttgatgatgctgctcaactttttatgttgaatgcctttgagaataattacgtagacattgagtggattaaattatcaaggaaggtgattagttatgctaaaggaattccattggctttgaaagttttgggctcctttctttatggcaaaagcaaggaagaatgggaaagccaacttgagaagctaaacaaaatgccacatgcgaaaattcaagatgtgatgagattgagttatgatcaactagatagggaagagaagaacattttcttatatattgcttgttttttgaaaggatatgaatcacaacaaataatacttctgttaaatgcctgtggtttctcaacaattattggattgagggtcctccgagacagagctctcataactgaagttaaagaatcagctatcagaaaatcaagaatatttatgcatgatttaatacaagaaatgggttgggaaattgttcgagaagaatcaattgataatcctgggaaccgtagtcgactatgggatgctaatgacatatatcatgtattaaaacataacatggggactgaagccattcaaagcataactctgaatgtggcaaaaattgaagatctgcagttaagtccaaaaactttgtcctcaatggacaatctgaaatttctcaatttttataaaggtgcttttgaaaaggggaatttacatcttcccagtggtcttgaatttttgtcgaatcagctcagacttttgcgttgggatgactacccttccaagtcattgccggcaacattttcggctgaaaacattgttactatgaaaatgtacaatagcaaacttacaaaactttgggatggtgtccagaacctcgtgaatttaagggagattcaacttgctgaatcagagtacctgattgagcttcctgatttttcagaggctaagcttcttaaaaatgtcaatcttagctattgtacaaggttgcgtagtgttcatccttctgttttaactctccccaatctcaaccaattgaggcttgtagattgcaaagcccttactagccttacaagcagtgcccctctaaaatcacttacatttctcgtgctcagtggatgttcgagactccgagagttttcagtaacatcagagaaaaggttagaactgtttctttcaaacacagccatcaatgatgaactgtggtcatcaagtggacgccttggcaacattgaagaattgagtgtagatggctgccaaagtctcacaaatctcccctacaagttagttgaccttagtggtcttcgttctcttagtatttctggttgcaacaagttagcatcaaatctgcatttcctattaaataagttacgcagtttgaaaaatttgtcactgaataattgtagcagcttatttgaactgcccgagaacattagtttcttgtcatcgttacgtgagttgtcactcacagggtcaactaatctgacaacactgcctgctagcatcaagcatcttttgaggctacaatgtcttaggttggacgaatgcaaaaggcttcaatccctgccagaacttccaccctctattgcacacttgtttgcaagggactgcacttccctcgagaccctaaagttaacttcaatgcctacagtctcattggaagatgaagtatgctttcactttggaaattgcatgaaattggatcggcaatcaattaaagctattgaggcaaaagcgctgcttgagatggataaattagtatcagctgttgaaacaggcactgcttggactatgcaggctcaacttgtatatccaggaagcaaagttccagaatggttcatgtacaggtcaacacagtcttccatcactgtggatctcttctccattccacgacctaaggattggagttttatattctgtgcagttgtttctcactctgaatccttcgtttactttgaaactgagtggtattttagtgatggccaacaggttttggatgagattatgtattacttttgcaaacatattggcatcattcagtcagatcatgtctgtctctggtatatctcagagcttccttcacaggtgcaaaggataattgaagagaagaatactgatcagagcaccactggtcatgaatcagttgaaataaaattcattgttaaaactcctagtccagttccagttttggatgtcaaggggtgtggggtatgcccaacgggtgcatcaggatatcgaaattatattcaacaaatggaatcggtgttacagtcacaacccaattcattgggaatcaaaggaggttgctcctgtgacaacactaatgacaaagaagtggttcctaggaagtccaaggattttatcttcccaagcccaccaactggggcgtcgaagaatagaacacgtggcttgaaagatattatgtttttatga

SEQ ID NO: 32 - protein, 1143 aa, Prosopis alba
MDSFSKLSGIQHDVFISFRGTDTRHGFLSHLRKELRQKQIDAYVDDRPESGDQISPALLRAIKESLISLIIFSKDYASSKWCLEELVQIIECMEKQKQIVIPVFYNTDPSNVRHQKGSYGDALSKHGEYYEERVPIWRSALKEAANLSGFHSSNYEDEAELIEEIAKSLSTKLNILYRGDLTDLVGIDERIAELESLMDEGSKDVRVIGIWGMGGIGKTTIAAAIFYRSCFEYEGHCFLANVREEAENHGIIYLKNKMLSSLLKENDLQIGTPYLMPPFVRRRLLRKRILVVLDDISDSDHLENLVGALDCFGSGSRVLITTRDKQVLAKRADEIYEVKPLMFDDAAQLFMLNAFENNYVDIEWIKLSRKVISYAKGIPLALKVLGSFLYGKSKEEWESQLEKLNKMPHAKIQDVMRLSYDQLDREEKNIFLYIACFLKGYESQQIILLLNACGFSTIIGLRVLRDRALITEVKESAIRKSRIFMHDLIQEMGWEIVREESIDNPGNRSRLWDANDIYHVLKHNMGTEAIQSITLNVAKIEDLQLSPKTLSSMDNLKFLNFYKGAFEKGNLHLPSGLEFLSNQLRLLRWDDYPSKSLPATFSAENIVTMKMYNSKLTKLWDGVQNLVNLREIQLAESEYLIELPDFSEAKLLKNVNLSYCTRLRSVHPSVLTLPNLNQLRLVDCKALTSLTSSAPLKSLTFLVLSGCSRLREFSVTSEKRLELFLSNTAINDELWSSSGRLGNIEELSVDGCQSLTNLPYKLVDLSGLRSLSISGCNKLASNLHFLLNKLRSLKNLSLNNCSSLFELPENISFLSSLRELSLTGSTNLTTLPASIKHLLRLQCLRLDECKRLQSLPELPPSIAHLFARDCTSLETLKLTSMPTVSLEDEVCFHFGNCMKLDRQSIKAIEAKALLEMDKLVSAVETGTAWTMQAQLVYPGSKVPEWFMYRSTQSSITVDLFSIPRPKDWSFIFCAVVSHSESFVYFETEWYFSDGQQVLDEIMYYFCKHIGIIQSDHVCLWYISELPSQVQRIIEEKNTDQSTTGHESVEIKFIVKTPSPVPVLDVKGCGVCPTGASGYRNYIQQMESVLQSQPNSLGIKGGCSCDNTNDKEVVPRKSKDFIFPSPPTGASKNRTRGLKDIMFL

SEQ ID NO: 33 - DNA, 570 bp, Phakopsora pachyrhizi
  source 1..570: mol_type = genomic DNA
atgttcatattgagttggttttgggacgttttagctcagttcggactggtaaataaaaatgcaaagatactgtttctggggttagacaatgccggtaaaactacgttgttacacatgctgaagaacgatcgactagcaactctccaaccaacacttcatccaacttcggaagagctggcgatcggtaacatcaaatttaccacttacgatctcggtggtcatcaacaggcccgaaggttatggaaggagtatttcccagaggtcgatgggatcgtcttcttagttgattctcaagacttggagagatttccagagtcaaaggctgagttggatgccttactcagtatagaggaactttttaaagtacccttcttgatcctaggtaacaagatcgatgccccaggtgccgtaagcgaggaagagctcaggcacgccctaggcctataccaaactaccggaaagggtaaagtgcccttgaccgatatcagaccgatcgagatcttcatgtgttcgatcgttatgagacaaggctatggtgacggttttcgttggttggcccagtacatttaa

SEQ ID NO: 34 - protein, 189 aa, Phakopsora pachyrhizi
MFILSWFWDVLAQFGLVNKNAKILFLGLDNAGKTTLLHMLKNDRLATLQPTLHPTSEELAIGNIKFTTYDLGGHQQARRLWKEYFPEVDGIVFLVDSQDLERFPESKAELDALLSIEELFKVPFLILGNKIDAPGAVSEEELRHALGLYQTTGKGKVPLTDIRPIEIFMCSIVMRQGYGDGFRWLAQYI

SEQ ID NO: 35 - protein, 1063 aa, Vigna unguiculata
MAVPSFSFSFTYDVFLSFRGEDTRYGFTGNLYRALRDRGIHTFIDDEELRKGDEITSALEKAIEGSRIFIIVFSLNYASSSFCLNELAYILPHAKRNALLVLPLFYDVVPSHVRHHTGSFGEALDAHEKRFRGMSQGFELNIEKLNKWKMALRRAANLSGYHFKHGEEYEYQFIERIVELVSKKINRAPLHVGDYPVGLEARVLEVKLLLEVGSDDVVHLVGIHGLGGIGKTTLALALYNSIADHFEGLCFLENVRENSNKHGLHHLQRILLSQVLGENNINITSVRQGISMMQHRLRQKKILLILDDVDKHEQLQAIVGRPDWFGPGSRVIITTRDKQLLSCHLVEKLYKVKKLEKNNALRLLSWKGFRTEEVDTSYLNVMDRVLAYASGHPLALEVIGSKLFSKSVKEWESAIKQYEKIPSNQILEVLKVSFDALEEVEKSVFLDIACCFKGYALSEIEDILRAHYGDCMKYHIGVLVEKSLMKYGYNSVVTLHDLIEDMGKEIVREKSPKNPGKRSRLWSPEDIIQVLEDNSGSGEIEIICLNSSLPDKEEIVGWNRKAFKKMKNLKTLIIKKGKFSEGPKYLPNSLRVLEWLKYPSQGLPPDFRSKKLAICKLPSSSFGSLELAEFSKKFMNMTLLNFDECEGLTHIPDVSGLPNLEKVSFKNCKSLVTIHDSFGFLGKLNSLSAVGCSKLRSFPPLKLTSLENLELSYCHSLESFPEILEKMGKITELVLEDCHIKELPFSFHNLTELQTLQLRWCPILRLPSSIVMMPKLAQIIAWESKGWLFPKQVDGEEKGSSMVSSNVDCLVLSGCKLSDDFFPVIPEWFSNVKDLDLSRNNFTVLPECISNCHSLCKLTLDSCHSLQEIRGIPPNIRHLSARNCKSFTSSCRSTLLNQKLHEAGNTMFWLSGAKFPEWFDHHGRGPSCSFWVGNKFPSIALCIAIGQTHIEQVEIVGPIMIINGIECSFDEEEDPYLYMLPHHTHIFDLQHIVFSDYLDRYVSENEWNHVEITYSVEQRFNKKDKHAVTPISIENGIYVLKQRSSMEDIQFTDPHKKRRLDVV

SEQ ID NO: 36 - protein, 1144 aa, Nicotiana glutinosa
MASSSSSSRWSYDVFLSFRGEDTRKTFTSHLYEVLNDKGIKTFQDDKRLEYGATIPGELCKAIEESQFAIVVFSENYATSRWCLNELVKIMECKTRFKQTVIPIFYDVDPSHVRNQKESFAKAFEEHETKYKDDVEGIQRWRIALNEAANLKGSCDNRDKTDADCIRQIVDQISSKLCKISLSYLQNIVGIDTHLEKIESLLEIGINGVRIMGIWGMGGVGKTTIARAIFDTLLGRMDSSYQFDGACFLKDIKENKRGMHSLQNALLSELLREKANYNNEEDGKHQMASRLRSKKVLIVLDDIDNKDHYLEYLAGDLDWFGNGSRIIITTRDKHLIEKNDIIYEVTALPDHESIQLFKQHAFGKEVPNENFEKLSLEVVNYAKGLPLALKVWGSLLHNLRLTEWKSAIEHMKNNSYSGIIDKLKISYDGLEPKQQEMFLDIACFLRGEEKDYILQILESCHIGAEYGLRILIDKSLVFISEYNQVQMHDLIQDMGKYIVNFQKDPGERSRLWLAKEVEEVMSNNTGTMAMEAIWVSSYSSTLRFSNQAVKNMKRLRVFNMGRSSTHYAIDYLPNNLRCFVCTNYPWESFPSTFELKMLVHLQLRHNSLRHLWTETKHLPSLRRIDLSWSKRLTRTPDFTGMPNLEYVNLYQCSNLEEVHHSLGCCSKVIGLYLNDCKSLKRFPCVNVESLEYLGLRSCDSLEKLPEIYGRMKPEIQIHMQGSGIRELPSSIFQYKTHVTKLLLWNMKNLVALPSSICRLKSLVSLSVSGCSKLESLPEEIGDLDNLRVFDASDTLILRPPSSIIRLNKLIILMFRGFKDGVHFEFPPVAEGLHSLEYLNLSYCNLIDGGLPEEIGSLSSLKKLDLSRNNFEHLPSSIAQLGALQSLDLKDCQRLTQLPELPPELNELHVDCHMALKFIHYLVTKRKKLHRVKLDDAHNDTMYNLFAYTMFQNISSMRHDISASDSLSLTVFTGQPYPEKIPSWFHHQGWDSSVSVNLPENWYIPDKFLGFAVCYSRSLIDTTAHLIPVCDDKMSRMTQKLALSECDTESSNYSEWDIHFFFVPFAGLWDTSKANGKTPNDYGIIRLSFSGEEKMYGLRLLYKEGPEVNALLQMRENSNEPTEHSTGIRRTQYNNRTSFYELING

(19) United States
(12) Patent Application Publication
(10) Pub. No.: US 2025/0230464 A1
(43) Pub. Date: Jul. 17, 2025

(54) PATHOGEN RESISTANCE IN PLANTS MEDIATED BY FIT2

(71) Applicant: Fortiphyte, Inc., Berkeley, CA (US)

(72) Inventor: Alexander Christiaan SCHULTINK, Berkeley, CA (US)

(21) Appl. No.: 18/853,637

(22) PCT Filed: Apr. 5, 2023
(86) PCT No.: PCT/US2023/065405

§ 371 Date: Oct. 2, 2024

Related U.S. Application Data
(60) Provisional application No. 63/328,050, filed on Apr. 6, 2022.

(51) Int. Cl. C12N 15/82; C07K 14/415; C12Q 1/6895

(52) U.S. Cl. CPC C12N 15/8282; C07K 14/415; C12N 15/8205; C12Q 1/6895; C12Q 2600/13

(57) ABSTRACT

The present disclosure provides an isolated, recombinant, or synthetic polynucleotide comprising a FIT2 protein, and homologs, fragments, and variations thereof. The disclosure further relates to transgenic plants, plant parts, and plant cells comprising one or more of these polynucleotides, and exhibit resistance or tolerance to a pathogen, such as fungal pathogens. The disclosure further relates to methods of genetically engineering a pathogen resistance or tolerance trait in a plant, plant part, or plant cell, comprising targeted gene editing of a FIT2 homolog or its regulatory elements to alter expression of a FIT2 homolog, and plants produced therefrom. The disclosure further relates to methods for identifying new functional FIT2 genes and/or alleles thereof.

### CROSS-REFERENCE TO RELATED APPLICATIONS

This application claims the benefit of U.S. Provisional Application No. 63/328,050 filed on Apr. 6, 2022, which is hereby incorporated by reference in its entirety for all purposes.

### STATEMENT REGARDING FEDERALLY SPONSORED RESEARCH OR DEVELOPMENT

This invention was made with Government support under 2112394 by the National Science Foundation. The Government has certain rights to this invention.

### REFERENCE TO AN ELECTRONIC SEQUENCE LISTING

The contents of the electronic sequence listing (FOTI_003_01WO_SeqList ST26.xml Size: 108,504 bytes; and Date of Creation: Mar. 29, 2023) are herein incorporated by reference in its entirety.

### TECHNICAL FIELD

The disclosure relates to the identification and use of nucleic acid sequences for pathogen resistance in plants.

### BACKGROUND

Plant pathogens are a major problem in modern agriculture, resulting in an estimated 10% decrease in crop productivity despite the widespread use of chemical controls. Chemical controls such as fungicides are expensive, do not always provide complete control of the target pathogen, and can have harmful impacts on human health and the environment. Fungal and oomycete pathogens are particularly problematic in agriculture. Foliar fungal and oomycete pathogens often spread by windblown spores and can rapidly infect a field to cause yield severe loss under conducive conditions. Spraying fungicide can help control foliar pathogens but is typically not effective for pathogens in the soil. Overuse of fungicides has resulted in many pathogens evolving tolerance, limiting the options for growers and potentially resulting in the use of more hazardous or more expensive chemicals. Approximately $20 billion of fungicide is sprayed each year and this number is expected to grow as agriculture continues to intensify and global warming leads to environmental conditions that tend to be more favorable for pathogens. There is an urgent need for crop varieties that have genetic resistance to problematic pathogen species.

An example of one such pathogen is the obligate biotrophic fungus Phakopsora pachyrhizi (and to a lesser extent, the closely related fungus Phakopsora meibomiae), which causes Asian soybean rust (ASR). While soybeans make up the primary commercial crop affected by ASR, Phakopsora infects leaf tissue from a broad range of leguminous plants, including at least 17 genera (Slaminko et al., 2008). In general, rust fungi (order Pucciniales) constitute one of the most economically important groups of plant pathogens because they spread quickly, they can cause severe yield loss, and most crops are susceptible to at least one rust species. There are more than 6000 species of rust fungi that cause harm to many plant species including wheat (Puccinia graminis and Puccinia triticina), common bean (Uromyces appendiculatus), soybean (Phakopsora pachyrhizi), sugar cane (Puccinia melanocephala) and coffee (Hemileia vastatrix) (Aime et al., 2006). Soil borne fungal pathogens, such as Fusarium and Verticillium, are difficult to control and once established in field require prolonged rotation away from susceptible crop species or aggressive soil fumigation to reduce pathogen populations. Oomycete pathogens, such as Phytophthora and Pythium, can cause severe yield loss and are also difficult to control. The use of crop varieties that have resistance or immunity to problematic pathogens is desirable over reliance on chemical controls, but for many pathogens such crop varieties are not available. In many cases plant disease resistance is mediated by specific immune receptor genes, which recognize specific proteins from invading pathogens and activate defense pathways to protect the plant. Immune receptor genes can be identified from plant species or accessions that have resistance to a particular pathogen of interest and can then be moved into a crop variety, by traditional breeding or other methods, to obtain a disease-resistant variety.

The foregoing examples of the related art and limitations related therewith are intended to be illustrative and not exclusive. Other limitations of the related art will become apparent to those of skill in the art upon a reading of the specification.

### SUMMARY

The present disclosure provides for an isolated, recombinant, or synthetic polynucleotide comprising a nucleic acid sequence encoding a functional FIT2 protein homologous to SEQ ID NO: 2. In some embodiments, the polynucleotide encodes a protein having at least 70% identity to SEQ ID NO: 2. In some aspects, the protein is selected from the group consisting of: SEQ ID NOs: 2, 4, 6, 8, 10, 12, 14, 16, 18, 20, 22, 24, 26, 28, 30, 32, and functional homologs thereof. In some aspects, the isolated, recombinant, or synthetic polynucleotide comprises a nucleic acid sequence selected from the group consisting of SEQ ID NOs: 1, 3, 5, 7, 9, 11, 13, 15, 17, 19, 21, 23, 25, 27, 29, 31, complements thereof, fragments thereof, and sequences at least 70% identical thereto. The disclosure further relates to genetic constructs comprising one or more of these sequences, and transgenic plants, plant parts, or plant cells comprising one or more of these sequences, wherein the plant, plant part, or plant cell is resistant or tolerant to a pathogen.

In another embodiment, the disclosure teaches a method of producing a plant, plant part, or plant cell having resistance or tolerance to a pathogen, wherein the method comprises transforming a plant, plant part, or plant cell with a polynucleotide encoding a functional FIT2 protein. In some aspects, the nucleotide sequence encoding the FIT2 protein has been codon optimized. In some aspects, the FIT2 protein comprises a selected from the group consisting of: SEQ ID NOs: 2, 4, 6, 8, 10, 12, 14, 16, 18, 20, 22, 24, 26, 28, 30, 32, and functional homologs thereof, or is encoded by an isolated, recombinant, or synthetic polynucleotide selected from the group consisting of SEQ ID NOs: 1, 3, 5, 7, 9, 11, 13, 15, 17, 19, 21, 23, 25, 27, 29, 31, complements thereof, fragments thereof, and sequences at least 70% identical thereto.

In another embodiment, the disclosure teaches a method of producing a plant, plant part, or plant cell having resistance or tolerance to a pathogen wherein the method comprises transforming a plant, plant part, or plant cell with a polynucleotide encoding a functional FIT2 protein along with additional genes required for TIR-NLR signaling or function, which may be missing or incompatible with FIT2 in the target plant.

In another embodiment, the disclosure teaches a method of genetically engineering a pathogen resistance or tolerance trait in a plant, plant part, or plant cell, comprising providing a plant species that is susceptible to a pathogen, identifying within the genome of the plant species a homolog of FIT2, wherein said homolog does not mediate AvrFIT2 recognition; and genetically modifying a plant, plant part, or plant cell from the susceptible plant species with targeted gene editing, wherein said targeted gene editing is directed at the FIT2 homolog, and wherein said targeted gene editing enables the FIT2 homolog to recognize AvrFIT2 and confers resistance or tolerance to a pathogen.

In another embodiment, the disclosure teaches a method of genetically engineering a pathogen resistance or tolerance trait in a plant, plant part, or plant cell, comprising providing a plant species that is susceptible to a pathogen, identifying within the genome of the plant species an endogenous FIT2 homolog, and genetically modifying a plant, plant part, or plant cell from the susceptible plant species with targeted gene editing to confer resistance or tolerance to a pathogen. In some cases, the modifying uses targeted gene editing to alter a regulatory sequence. In some cases, the modifying involves adding a regulatory element. In some embodiments, the targeted gene editing restores function of the endogenous FIT2 homolog. In some embodiments, the targeted gene editing increases expression of the endogenous FIT2 homolog.

In another embodiment, the disclosure relates to recombinant DNA constructs comprising at least one of: a nucleic acid sequence encoding a protein comprising SEQ ID NO: 2, or a protein at least 90% identical thereto; a nucleic acid sequence encoding a protein comprising SEQ ID NO: 4, or a protein at least 90% identical thereto; a nucleic acid sequence encoding a protein comprising SEQ ID NO: 6, or a protein at least 90% identical thereto; a nucleic acid sequence encoding a protein comprising SEQ ID NO: 8, or a protein at least 90% identical thereto; a nucleic acid sequence encoding a protein comprising SEQ ID NO: 10, or a protein at least 90% identical thereto; a nucleic acid sequence encoding a protein comprising SEQ ID NO: 12, or a protein at least 90% identical thereto; a nucleic acid sequence encoding a protein comprising SEQ ID NO: 14, or a protein at least 90% identical thereto; a nucleic acid sequence encoding a protein comprising SEQ ID NO: 16, or a protein at least 90% identical thereto; a nucleic acid sequence encoding a protein comprising SEQ ID NO: 18, or a protein at least 90% identical thereto; a nucleic acid sequence encoding a protein comprising SEQ ID NO: 20, or a protein at least 90% identical thereto; a nucleic acid sequence encoding a protein comprising SEQ ID NO: 22, or a protein at least 90% identical thereto; a nucleic acid sequence encoding a protein comprising SEQ ID NO: 24, or a protein at least 90% identical thereto; a nucleic acid sequence encoding a protein comprising SEQ ID NO: 26, or a protein at least 90% identical thereto; a nucleic acid sequence encoding a protein comprising SEQ ID NO: 28, or a protein at least 90% identical thereto; a nucleic acid sequence encoding a protein comprising SEQ ID NO: 30, or a protein at least 90% identical thereto; a nucleic acid sequence encoding a protein comprising SEQ ID NO: 32, or a protein at least 90% identical thereto; or combinations thereof, and wherein the construct is capable of conferring resistance to a fungal pathogen when transformed into a plant.

In another embodiment, the disclosure relates to transgenic plants, plant parts, or plant cells having resistance or tolerance to a fungal pathogen, wherein the resistance or tolerance is conferred by a transgene comprising at least one of: a nucleic acid sequence encoding a protein comprising SEQ ID NO: 2, or a protein at least 90% identical thereto; a nucleic acid sequence encoding a protein comprising SEQ ID NO: 4, or a protein at least 90% identical thereto; a nucleic acid sequence encoding a protein comprising SEQ ID NO: 6, or a protein at least 90% identical thereto; a nucleic acid sequence encoding a protein comprising SEQ ID NO: 8, or a protein at least 90% identical thereto; a nucleic acid sequence encoding a protein comprising SEQ ID NO: 10, or a protein at least 90% identical thereto; a nucleic acid sequence encoding a protein comprising SEQ ID NO: 12, or a protein at least 90% identical thereto; a nucleic acid sequence encoding a protein comprising SEQ ID NO: 14, or a protein at least 90% identical thereto; a nucleic acid sequence encoding a protein comprising SEQ ID NO: 16, or a protein at least 90% identical thereto; a nucleic acid sequence encoding a protein comprising SEQ ID NO: 18, or a protein at least 90% identical thereto; a nucleic acid sequence encoding a protein comprising SEQ ID NO: 20, or a protein at least 90% identical thereto; a nucleic acid sequence encoding a protein comprising SEQ ID NO: 22, or a protein at least 90% identical thereto; a nucleic acid sequence encoding a protein comprising SEQ ID NO: 24, or a protein at least 90% identical thereto; a nucleic acid sequence encoding a protein comprising SEQ ID NO: 26, or a protein at least 90% identical thereto; a nucleic acid sequence encoding a protein comprising SEQ ID NO: 28, or a protein at least 90% identical thereto; a nucleic acid sequence encoding a protein comprising SEQ ID NO: 30, or a protein at least 90% identical thereto; a nucleic acid sequence encoding a protein comprising SEQ ID NO: 32, or a protein at least 90% identical thereto; or combinations thereof. In some embodiments, the transgenic plants, plant parts, or plant cells further comprise one or more TIR-NLR signaling transgenes.

The disclosure further relates to genetically modified plants, plant parts, or plant cells produced by the methods disclosed herein, wherein the plant, plant part, or plant cell exhibits resistance or tolerance to a pathogen. In some aspects, the pathogen is a fungus or Oomycete.

In some aspects, the pathogen is Basidiomycete or Ascomycete. In some aspects, the pathogen is from the order Cantharellales, Mucorales, Ustilaginales, Atheliales, or Pucciniales. In some aspects, the fungal pathogen is Verticillium spp., Colletotrichum spp., Fusarium spp., Sclerotinia spp., Blumeria spp., Corynespora spp., Alternaria spp., Macrophomina spp., Cercospora spp., Fulvia spp., Choanephora spp., Ustilago maydis, Athelia spp., Rhizoctonia solani, Melampsora spp., Phakopsora pachyrhizi, Phakopsora meibomiae, Phakopsora euvitis, Phakopsora spp., Puccinia spp., Uromyces spp., Austropuccinia spp., Cronartium spp., Hemileia vastatrix, Albugo spp., Phytophthora spp., Pythium spp., or Peronospora spp.

In some aspects, the plant, plant part, or plant cell is in the subfamily Papilionoideae. In some aspects, the plant, plant part, or plant cell is soybean (Glycine max), peanut (Arachis hypogaea), coffee (Coffea spp.), Brassica spp. (e.g., Brassica napus, Brassica rapa, Brassica juncea), cotton (Gossypium barbadense, Gossypium hirsutum), cannabis (Cannabis spp.), potato (Solanum tuberosum), tomato (Solanum lycopersicum), eggplant (Solanum melongena), cocoa (Theobroma cacao), wheat (Triticum aestivum), corn (Zea mays), rice (Oryza sativa), banana (Musa spp.), sugarcane (Saccharum spp.), oats (for example Avena sativa), barley (for example Hordeum vulgare), sugar beets (Beta vulgaris), sunflower (Helianthus annuus), spinach (Spinacia oleracea), melon (Cucumis melo), avocado (Persea americana), mango (Mangifera indica), Pisum spp., Phaseolus spp., Medicago spp., or Vigna spp.

The disclosure further relates to pants, plant parts, or plant cells that have been genetically engineered to increase expression of an endogenous FIT2 gene. In some embodiments, the endogenous FIT2 gene sequence is inserted into a native, non-essential gene.

The disclosure further relates to methods for identifying a functional FIT2 gene and/or allele thereof comprising isolating a FIT2 homolog or allele thereof; expressing said FIT2 homolog or allele thereof in combination with an AvrFIT2 protein in a plant, plant part, or plant cell; and assaying said plant, plant part, or plant cell for an immune response. In some aspects, the AvrFIT2 protein comprises SEQ ID NO: 34, or sequences at least 60% identical thereto.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 shows a phylogenetic tree of AvrFIT2 homologs identified by performing a BLAST® search of the NCBI protein database to Phakopsora pachyrhizi AvrFIT2 (SEQ ID NO: 34). The AvrFIT2 protein is highly conserved in diverse fungal and oomycete species. The obtained sequences were aligned using Clustal Omega and manually filtered to remove redundant and incomplete sequences. A putative ortholog of AvrFIT2 was found in all fungal and Oomycete genomes examined, but only primarily alleles from pathogenic species were included in the tree to show a range of AvrFIT2 diversity in pathogen species across these clades. A maximum likelihood phylogenetic tree was constructed from the aligned protein sequences.

FIGS. 2A-2D shows photographs of plants inoculated with Phakopsora spores. At 17 days post inoculation, Glycine max (FIG. 2C-2D) had many lesions with abundant fungal spores visible. No spores or disease lesions are visible on Crotalaria juncea (FIG. 2A-2B).

FIG. 3A shows a phylogenetic tree of homologs of Crotalaria juncea (CjFIT2) (SEQ ID NO: 2) identified by performing a BLAST® search and conducting a protein alignment. This figure shows putative FIT2 orthologs in Lupinus angustifolius (LanFIT2) XP_019436492.1_corrected (SEQ ID NO: 4), Lupinus albus (LaFIT2) Lqlb_Chr22g0351831_corrected, (SEQ ID NO: 6), Spatholobus suberectus (SsFIT2) TKY48271.1 (SEQ ID NO: 8), Cajanus cajan (CcFIT2) XP_020223666.1 (SEQ ID NO: 10), Mucuna pruriens (MpFIT2) RDX76676.1 (SEQ ID NO: 12), Glycine soja (GsFIT2) GlysoPI483463.15G023000.1 (SEQ ID NO: 14), Glycine max (GmFIT2) GlymaLeel5g024800 (SEQ ID NO: 16), Trifolium pratense (TpFIT2) XP_045799977.1 (SEQ ID NO: 18), Medicago truncatula (MtFIT2) Medtr2g099920.1 (SEQ ID NO: 20), Lotus japonicus (LjFIT2) Lj3g0010719.1 (SEQ ID NO: 22), Prosopis alba XP_028800138.1 (SEQ ID NO: 24), Prosopis alba XP_028802438.1 (SEQ ID NO: 26), Prosopis alba XP_028763131.1 (SEQ ID NO: 28), Prosopis alba XP_028774409.1 (SEQ ID NO: 30), and Prosopis alba XP_028756320.1 (SEQ ID NO: 32). The phylogenetic tree was rooted using paralogs of FIT2 that do not function in AvrFIT2 perception.

FIG. 3B shows a protein alignment of the amino acid sequences for CjFIT2 (SEQ ID NO: 2), Vigna unguiculata (VuFIT1) (Vu01g041300.1) (SEQ ID NO: 35), and the N gene (SEQ ID NO: 36), which gives TMV resistance. The TIR domain is underlined in red, the NB-ARC domain is underlined in blue, and the LRR domain is underlined in yellow.

FIGS. 4A and 4B show bar graphs of the average transcript coverage for the top 30 soybean NLR transcripts (FIG. 4A) and top 30 C. juncea transcripts (FIG. 4B). GmFIT2 is poorly expressed with an average coverage more than 30-fold lower than the average coverage of the top 30 soybean NLR genes (FIG. 4A), whereas CjFIT2 is the₂₅₁ₕ most expressed NLR gene (FIG. 4B).

FIG. 5 shows a map of an example DNA construct comprising CjFIT2 (SEQ ID NO: 1) that can be used for transformation of a plant cell, selection of transformed cells, and expression of FIT2.

FIG. 6 shows results of transient expression of FIT2 and PpAvrFIT2 (AvrFIT2 from Phakopsora pachyrhizi) in Nicotiana tabacum leaf tissue (lacking endogenous FIT2). As shown on the right side of the leaf, expression of FIT2+AvrFIT2 leads to a strong cell death response, known as a hypersensitive response, that indicates strong immune activation. This response is not observed when FIT2 is expressed with AvrFITla or when AvrFIT2 is expressed with VuFIT1 (FIT1 from Vigna unguiculata) (left side of the leaf).

FIG. 7 depicts the results of various FIT2 alleles transiently expressed in Nicotiana tabacum leaf tissue (lacking an endogenous FIT2). Expression of PpAvrFIT2 alone or CjFIT2 alone triggers no response, but co-expression of these genes triggers a strong hypersensitive cell death response indicating immune activation. FIT2 alleles from Glycine max (GmFIT2) and Lupinus albus (LaFIT2) both show the ability to recognize PpAvrFIT2.

FIG. 8 depicts the results of various AvrFIT2 alleles co-expressed with CjFIT2 (SEQ ID NO: 1). CjFIT2 and the AvrFIT2 alleles were expressed alone or in combination in Nicotiana tabacum leaf tissue. A localized hypersensitive cell death response indicates that the receptor can recognize the ligand protein. In addition to recognizing AvrFIT2 from Phakopsora pachyrhizi, the CjFIT2 can recognize AvrFIT2 proteins from other species including Melampsora larici-populina and Puccinia graminis.

FIG. 9 shows the recognition spectra of FIT2 alleles. Alleles of FIT2 (top row) were co-expressed with alleles of AvrFIT2 (first column) in Nicotiana tabacum leaf tissue. After three days, each infiltration was scored for the presence of a hypersensitive cell death response indicative of immune activation and a compatible interaction between the FIT2+AvrFIT2 alleles. Grey=strong hypersensitive response, striped=moderate or weak hypersensitive response, dots=no visible response. Crotalaria juncea (CjFIT2), Glycine max (GmFIT2), Lupinus albus (LaFIT2), Spatholobus suberectus (SsFIT2), Cajanus cajan (CcFIT2), Mucuna pruriens (MpFIT2), Lotus japonicus (LjFIT2), Trifolium pratense (TpFIT2), and Medicago truncatula (MtFIT2).

FIGS. 10A-10D depict soybean leaves expressing CjFIT2 (FIG. 10A, close up shown in FIG. 10B) and wild type soybean leaves lacking CjFIT2 (FIG. 10C, close up shown in FIG. 10D) inoculated with Phakopsora pachyrhizi. The leaves were photographed at 25 days post inoculation.

### DETAILED DESCRIPTION

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Following long-standing patent law convention, the terms “a,” “an,” and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of ±10% from the specified amount, as such variations are appropriate to perform the disclosed methods and/or employ the disclosed compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one or more of the elements to which the “and/or” refers can also individually be present in single or multiple occurrences in the combinations(s) and/or subcombination(s).

As used herein, the term “plant” can refer to any living organism belonging to the kingdom Plantae (i.e., any genus/species in the Plant Kingdom), to a whole plant, any part thereof, or a cell or tissue culture derived from a plant. Thus, the term “plant” can refer to any of whole plants, plant components or organs (e.g., leaves, stems, roots, etc.), plant tissues, seeds and/or plant cells.

A plant cell is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores.

The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “resistant”, or “resistance”, describes a plant, line or variety that shows fewer or reduced symptoms to a biotic pest or pathogen than a susceptible (or more susceptible) plant, line or variety to that biotic pest or pathogen. This term is also applied to plants that show no symptoms, and may also be referred to as “high/standard resistance”.

As used herein, the term “tolerant” or “tolerance” describes a plant, line, or variety that that shows some symptoms to a biotic pest or pathogen, but that are still able to produce marketable product with an acceptable yield. These lines may also be referred to as having “moderate/intermediate resistance”. Tolerant and moderate/intermediate resistant plant types restrict the growth and development of the specified pest or pathogen, but exhibit a greater range of symptoms or damage compared to plant types with high resistance. Plant types with intermediate resistance will show less severe symptoms than susceptible plant varieties, when grown under similar field conditions and pathogen pressure. A “tolerant” plant may also indicate a phenotype of a plant wherein disease-symptoms remain absent upon exposure of said plant to an infective dosage of pathogen, whereby the presence of a systemic or local pathogen infection, pathogen multiplication, at least the presence of pathogen genomic sequences in cells of said plant and/or genomic integration thereof can be established.

Tolerant plants are therefore resistant for symptom expression but symptomless carriers of the pathogen. Sometimes, pathogen sequences may be present or even multiply in plants without causing disease symptoms. This phenomenon is also known as “latent infection”. In latent infections, the pathogen may exist in a truly latent non-infectious occult form, possibly as an integrated genome or an episomal agent (so that pathogen protein cannot be found in the cytoplasm, while PCR protocols may indicate the present of pathogen nucleic acid sequences) or as an infectious and continuously replicating agent. A reactivated pathogen may spread and initiate an epidemic among susceptible contacts. The presence of a “latent infection” is indistinguishable from the presence of a “tolerant” phenotype in a plant.

Methods of evaluating resistance are well known to one skilled in the art. Such evaluation may be performed by visual observation of a plant or a plant part (e.g., leaves, roots, flowers, fruits et. al) in determining the severity of symptoms. For example, when each plant is given a resistance score on a scale of 1 to 5 based on the severity of the reaction or symptoms, with 1 being the resistance score applied to the most resistant plants (e.g., no symptoms, or with the least symptoms), and 5 the score applied to the plants with the most severe symptoms, then a line is rated as being resistant when at least 75% of the plants have a resistance score at a 1, 2, or 3 level, while susceptible lines are those having more than 25% of the plants scoring at a 4 or 5 level. If a more detailed visual evaluation is possible, then one can use a scale from 1 to 10 so as to broaden out the range of scores and thereby hopefully provide a greater scoring spread among the plants being evaluated. Additional methods for evaluating resistance are well known in the art (see for example, jircas.go.jp/sites/default/files/publication/manual_guideline/manual_guideline-_—73.pdf available on the world wide web).

In addition to such visual evaluations, disease evaluations can be performed by determining the pathogen bio-density in a plant or plant part using electron and/or light microscopy and/or through molecular biological methods, such as protein quantification (e.g., ELISA, measuring pathogen protein density) and/or nucleic acid quantification (e.g., RT-PCR, measuring pathogen RNA density). Another method relies on quantifying the spores produced by the pathogen, which can be quantified using a hemacytometer and evaluated per uredinium, per leaf area, or per leaf.

As used herein, the term “susceptible” is used herein to refer to a plant that is unable to prevent entry of the pathogen into the plant and/or slow multiplication and systemic spread of the pathogen, resulting in disease symptoms. The term “susceptible” is therefore equivalent to “non-resistant”.

As used herein, the term “homologous” or “homolog” is used as it is known in the art and refers to related sequences that share a common ancestor. The term “homolog” is sometimes used to apply to the relationship between genes separated by the event of speciation (“ortholog”) or to the relationship between genes separated by the event of genetic duplication within the same species (“paralog”). Homology can be determined using software programs readily available in the art, such as those discussed in Current Protocols in Molecular Biology (F. M. Ausubel et al., eds., 1987) Supplement 30, section 7.718, Table 7.71.

As used herein, the term “allele” is used both as it is known in the art as one of two or more versions of a gene or peptide, and also to refer to synthetic variants of a gene or peptide containing one or more changes from the native sequence.

As used herein, the term “functional” used in the context of a homolog means that the homolog has the same or very similar function. For example, a functional homolog of FIT2 would recognize an AvrFIT2 protein. A “nonfunctional FIT2 homolog” would not recognize AvrFIT2, though it may still be functional in that it is able recognize other pathogen-derived proteins.

As used herein, the term “sequence identity” refers to the presence of identical nucleotides or amino acids at corresponding positions of two sequences. Readily available sequence comparison and multiple sequence alignment algorithms are, respectively, the Basic Local Alignment Search Tool (BLAST®) and ClustalW/ClustalW2/Clustal Omega programs available on the Internet (e.g., the website of the EMBL-EBI). Some alignment programs are MacVector (Oxford Molecular Ltd, Oxford, U.K.) and ALIGN Plus (Scientific and Educational Software, Pennsylvania). Other non-limiting alignment programs include Sequencher (Gene Codes, Ann Arbor, Michigan), AlignX, and Vector NTI (Invitrogen, Carlsbad, CA). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001. Unless otherwise noted, alignments disclosed herein utilized Clustal Omega.

As used herein, the phrases “DNA construct”, “expression cassette”, “chimeric construct”, “construct”, and “recombinant DNA construct” are used interchangeably herein. A recombinant DNA construct comprises an artificial combination of nucleic acid fragments, e.g., regulatory and coding sequences that are not found together in nature. For example, a construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746).

As used herein “cisgene” refers to a gene from the same species, or a species closely related enough to be conventionally bred. “Transgene” refers to a gene from a different species, and may also be referred to as “heterologous” (an amino acid or a nucleic acid sequence which is not naturally found in the particular organism). Both transgenes and heterologous sequences would be considered “exogenous” as referring to a substance coming from some source other than its native source.

The term “operably linked” refers to the juxtaposition of two or more components (such as sequence elements) having a functional relationship. For example, the sequential arrangement of the promoter polynucleotide with a further oligo- or polynucleotide, resulting in transcription of the further polynucleotide.

As used herein, “promoter” refers to a DNA sequence capable of controlling the expression of a coding sequence or functional RNA. In some embodiments, the promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter.

As used herein, “selectable marker” is a nucleic acid segment that allows one to select for a molecule (e.g., a plasmid) or a cell that contains it, often under particular conditions.

These markers can encode an activity, such as, but not limited to, production of RNA, peptide, or protein, or can provide a binding site for RNA, peptides, proteins, inorganic and organic compounds or compositions and the like.

As used herein, “genetically engineered” refers to a plant, plant part, or plant cell which has been modified using genetic engineering methods.

As used herein, “transgenic” refers to a plant, plant part, or plant cell whose genome has been altered by the introduction of an exogenous DNA sequence by artificial means.

As used herein, a “non-essential gene” is a gene which is not required for the survival of a plant, plant part, or plant cell.

The following description includes information that may be useful in understanding the present disclosure. It is not an admission that any of the information provided herein is prior art or relevant to the presently claimed disclosures, or that any publication specifically or implicitly referenced is prior art.

### Overview

The present disclosure provides an isolated, recombinant, or synthetic polynucleotide comprising a FIT2 protein, and homologs, fragments, and variations thereof. The disclosure further relates to plants, plant parts, and plant cells that have been transformed with these polynucleotides, and exhibit resistance or tolerance to a plant pathogen, such as Phakopsora pachyrhizi. The disclosure further relates to methods of identifying pathogen resistant genes, and methods of genetically engineering a pathogen resistance or tolerance trait in a plant, plant part, or plant cell, comprising targeted gene editing of a homolog (such as FIT2), and plants produced therefrom.

### Plant Pathogens

There are more than 6000 species of rust fungi, including for example, Phakopsora pachyrhizi, Puccinia spp., Uromyces spp., Austropuccinia psidii, Cronartium spp., Melampsora larici-populina, and Hemileia vastatrix, that infect a wide range of important crops and ornamental varieties. Some examples of varieties that may be infected include, but are not limited to, Avena sativa (oats), Vicia faba (broad beans), Coffea arabica (coffee), Chrysanthemum, Cydonia (quince), Fuchsia spp. (garlic), Hordeum vulgare (barley), Juniperus virginiana (red cedar), Juniperus communis (juniper), Allium ampeloprasum (leek), Malus spp. (apple), Mentha piperita (peppermint), Mespilus (medlar), Onion, Pelargonium, Primula vulgaris (primrose), Pyrus (pear), Rosa spp. (roses), Triticum spp. (wheat), Secale cereale (rye), Vitis vinfera (grape), Saccharum spp. (sugarcane), Pinus spp. (pine), Myrtaceae, Populus spp. (poplars), and Glycine max (soybean).

In soybean, rust pathogen infections have been reported in South America, Africa, Australia, and Asia, and were first reported on soybean crops in the southern United States in 2004. Asian soybean rust (ASR), caused by Phakopsora pachyrhizi, spreads quickly and can lead to significant yield loss. Initial symptoms of ASR include yellow discoloration on the upper surfaces of foliage, followed by tan or reddish-brown lesions on the undersides of the leaves and sometimes on petioles, stems or pods. Blisters develop within the lesions, which break open and release spores. Soybean plants infected with ASR will exhibit reduced pod production and can result in a yield loss of greater than 50%. Another disease, New World soybean rust, caused by Phakopsora meibomiae, is generally not as harmful as ASR. P. meibomiae has not yet been reported in the U.S.

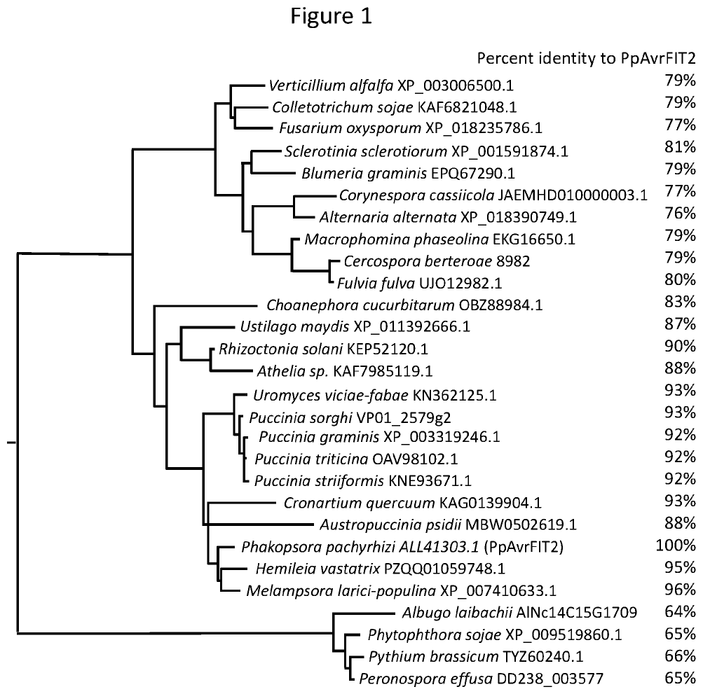

Successful infection of rust fungi relies on the secretion of effector proteins with functions that facilitate host colonization. The effector proteins suppress plant immunity and manipulate the host metabolism to benefit the pathogen (Jones and Dangl, 2006). AvrFIT2 is not an effector protein but is present in in the Phakopsora pachyrhizi pathogen that causes ASR. AvrFIT2 is present in all publicly accessible sequenced Phakopsora pachyrhizi strains isolated from various locations across the world, including Brazil and North America. A phylogenetic tree of close homologs of PpAvrFIT2 reveals that AvrFIT2 is highly conserved in diverse fungal and oomycete species (FIG. 1).

For example, AvrFIT2 is present in Rhizoctonia solani, a non-rust pathogen that can be problematic for herbaceous plants, causing diseases such as collar rot, root rot, damping off, and wire stem. AvrFIT2 is present in Choanephora cucurbitarum, a pathogen that causes fruit and blossom rot in various cucurbits. AvrFIT2 is present in Ustilago maydis, a smut fungus that infects maize. AvrFIT2 is present in Athelia, corticioid fungi that can be facultative parasites of crops.

AvrFIT2 is a putative ortholog of SAR1, an Arf family GTPase that is involved in vesicle formation and is required for protein secretion (Donaldson et al., 2011; Hernández-Gonzilez et al., 2014). SAR1 has been shown to be an essential gene in several fungal species, which is consistent with the broad conservation observed for AvrFIT2 in fungi and oomycetes. Due to the essential nature and high conservation of AvrFIT2, it is evolutionarily advantageous for plants to have evolved FIT2 specifically to mediate recognition of this protein. Fungal pathogens are unable to evade immunity mediated by FIT2 by losing AvrFIT2 because such a gene loss would likely be lethal. AvrFIT2/SAR1 is not a canonical effector protein, but rather is a cytoplasmic protein that associates with the endoplasmic reticulum during COPII-vesicle formation. As FIT2 is a putative intracellular immune receptor, a mechanism presumably exists for AvrFIT2/SAR1 to get delivered to the plant cytoplasm. Although still are area of active research, exosomes (extracellular vesicles) likely play a significant role in the delivery of virulence factors, including proteins and small RNAs, from fungal pathogens to plant cells (Presti et al., 2017; Cai et al., 2019). Exosome contents are derived from the cytoplasm and proteomic analysis routinely identifies cytoplasmic proteins in them (Schorey et al., 2014; Bleackley et al., 2019). Indeed, AvrFIT2/SAR1 has been detected in the extracellular vesicles of the plant pathogen Colletotrichum higginsianum (Rutter et al., 2022). AvrFIT2/SAR1 may have a direct role in the formation of fungal exosomes that are delivered into the plant cell. Alternatively, AvrFIT2/SAR1 may be unintentionally incorporated into exosomes as part of the exosome formation process which involves invagination of vesicles to form multi-vesicular bodies. Regardless of the delivery pathway, detection of AvrFIT2/SAR1 by FIT2 is a clear signal to the plant that it is being colonized by a fungus or oomycete and allows for appropriate immune activation to prevent disease.

### Plant Resistance

Plants have, in some cases, evolved immunity in which resistance gene products recognize the activity of specific effectors resulting in effector-trigger immunity (ETI) (Jones and Dangl, 2006). ETI leads to robust defenses, such as the hypersensitive response (HR), which is a form of programmed cell death that results in the formation of a localized lesion that inhibits pathogen growth at the initial infection site (Dodds and Rathjen, 2010). If the plant has an immune receptor capable of recognizing the pathogen effector protein, the effector protein activates a strong immune response conferring immunity. The perception of intracellular pathogen effector proteins in plants is frequently mediated by proteins from a large gene family known as the nucleotide binding, leucine-rich repeat (NLR) proteins (Jones et al., 2016).

Plant disease resistance traits are often encoded by NLR genes. NLR genes can be incorporated into a susceptible crop variety to confer resistance through a variety of methods including introgression breeding, transformation or genome editing. A typical plant has hundreds of NLR immune receptor genes (Jones et al., 2016). These genes are typically expressed at relatively low levels with the NLR proteins passively surveilling for the presence of cognate effector proteins from invading pathogens. Prior to activation, the NLR proteins have essentially no impact on plant metabolism or growth. Upon activation by a cognate ligand, typically a pathogen effector protein or a protein substrate of an effector, the NLR protein initiates a signalling cascade that activates endogenous plant defense pathways to inhibit pathogen growth. Using NLRs is a natural, safe, and environmentally sustainable mechanism to develop disease-resistant crop varieties to improve plant yields and reduce the need for chemical controls.

### FIT2

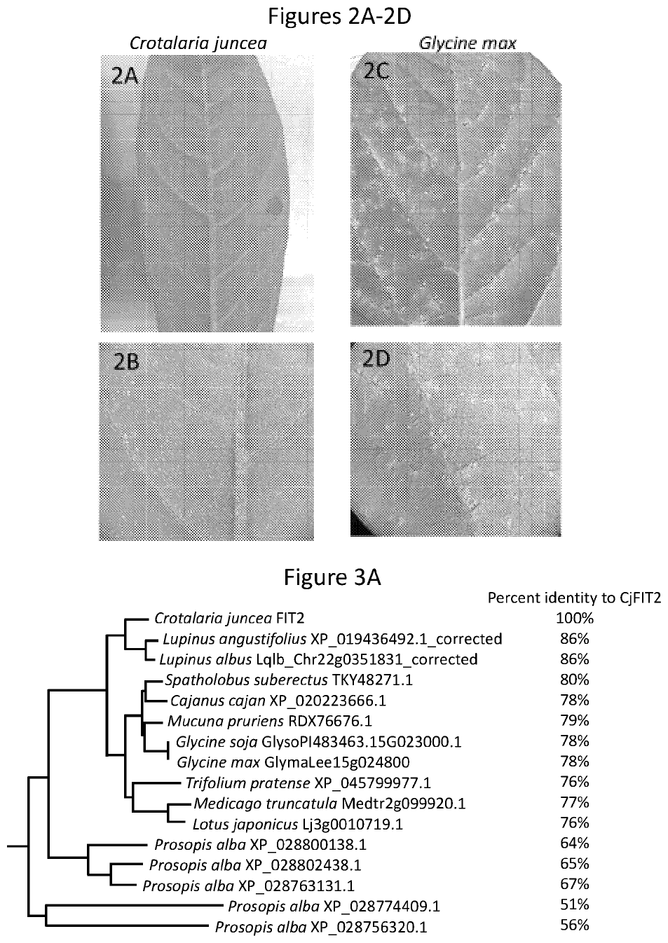

FIT2 is a plant Toll-like interleukin-1 receptor (TIR) nucleotide binding leucine rich repeat (NLR) immune receptor protein discovered by Applicants. It was identified from Crotalaria juncea (commonly known as brown hemp, Indian hemp, Madras hemp, or sunn hemp) and is responsible for AvrFIT2 recognition. As shown in FIGS. 2A and 2B, endogenous expression of FIT2 in C. juncea correlates with resistance to ASR. Leaves from C. juncea and Glycine max were inoculated with Phakopsora pachyrhizi. At 17 days post inoculation, Glycine max had many lesions with abundant fungal spores visible (FIGS. 2C and 2D). No spores or disease lesions were visible on Crotalaria juncea (FIGS. 2A and 2B). These results indicate a correlation between expression of FIT2 and resistance to Phakopsora pachyrhizi and suggest that expression of FIT2 in plants can confer disease resistance. Additionally, the widespread distribution of AvrFIT2 discussed above suggests that FIT2 can confer disease resistance against a broad range of pathogenic species. Although NLRs are typically associated with Effector Triggered Immunity (ETI), AvrFIT2 is not an effector protein and is highly conserved even in non-pathogenic fungi. AvrFIT2/SAR1 acts as a so called Microbial Associated Molecular Pattern (MAMP), the detection of which indicates invasion of the plant by a fungal or Oomycete species.

Thus, an embodiment of the present disclosure provides an isolated, recombinant, or synthetic polynucleotide comprising a nucleic acid sequence encoding a FIT2 protein, wherein the protein is selected from the group consisting of: SEQ ID Nos: 2, 4, 6, 8, 10, 12, 14, 16, 18, 20, 22, 24, 26, 28, 30, 32, functional homologs, and/or fragments and variations thereof. In some cases, the functional FIT2 homolog shares at least about 70% identity to SEQ ID NO: 2 and recognizes an AvrFIT2 protein secreted by a plant pathogen. In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 2.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 4.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 6.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 8.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 10.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 12

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 14.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 16.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 18.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 20.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 22.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 24.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 26.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 28.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 30.

In some cases, the functional FIT2 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 32.

In some aspects, the isolated, recombinant, or synthetic polynucleotide comprises a nucleic acid sequence encoding SEQ ID NO: 2, or an amino acid sequence at least 90% identical thereto. In some aspects, the isolated, recombinant, or synthetic polynucleotide comprises a nucleic acid sequence encoding SEQ ID NO: 4, or an amino acid sequence at least 90% identical thereto. In some aspects, the isolated, recombinant, or synthetic polynucleotide comprises a nucleic acid sequence encoding SEQ ID NO: 6, or an amino acid sequence at least 90% identical thereto. In some aspects, the isolated, recombinant, or synthetic polynucleotide comprises a nucleic acid sequence encoding SEQ ID NO: 8, or an amino acid sequence at least 90% identical thereto. In some aspects, the isolated, recombinant, or synthetic polynucleotide comprises a nucleic acid sequence encoding SEQ ID NO: 10, or an amino acid sequence at least 90% identical thereto. In some aspects, the isolated, recombinant, or synthetic polynucleotide comprises a nucleic acid sequence encoding SEQ ID NO: 12, or an amino acid sequence at least 90% identical thereto. In some aspects, the isolated, recombinant, or synthetic polynucleotide comprises a nucleic acid sequence encoding SEQ ID NO: 14, or an amino acid sequence at least 90% identical thereto. In some aspects, the isolated, recombinant, or synthetic polynucleotide comprises a nucleic acid sequence encoding SEQ ID NO: 16, or an amino acid sequence at least 90% identical thereto. In some aspects, the isolated, recombinant, or synthetic polynucleotide comprises a nucleic acid sequence encoding SEQ ID NO: 18, or an amino acid sequence at least 90% identical thereto. In some aspects, the isolated, recombinant, or synthetic polynucleotide comprises a nucleic acid sequence encoding SEQ ID NO: 20, or an amino acid sequence at least 90% identical thereto. In some aspects, the isolated, recombinant, or synthetic polynucleotide comprises a nucleic acid sequence encoding SEQ ID NO: 22, or an amino acid sequence at least 90% identical thereto. In some aspects, the isolated, recombinant, or synthetic polynucleotide comprises a nucleic acid sequence encoding SEQ ID NO: 24, or an amino acid sequence at least 90% identical thereto. In some aspects, the isolated, recombinant, or synthetic polynucleotide comprises a nucleic acid sequence encoding SEQ ID NO: 26, or an amino acid sequence at least 90% identical thereto. In some aspects, the isolated, recombinant, or synthetic polynucleotide comprises a nucleic acid sequence encoding SEQ ID NO: 28, or an amino acid sequence at least 90% identical thereto. In some aspects, the isolated, recombinant, or synthetic polynucleotide comprises a nucleic acid sequence encoding SEQ ID NO: 30, or an amino acid sequence at least 90% identical thereto. In some aspects, the isolated, recombinant, or synthetic polynucleotide comprises a nucleic acid sequence encoding SEQ ID NO: 32, or an amino acid sequence at least 90% identical thereto.

In another embodiment, the disclosure relates to a transgenic plant, plant part, or cell having resistance or tolerance to at least one plant pathogen, wherein the resistance or tolerance is conferred by a polynucleotide encoding at least one of the functional FIT2 homologs disclosed herein.

In another embodiment, the present disclosure provides an isolated, recombinant, or synthetic polynucleotide, wherein the polynucleotide comprises a nucleic acid sequence selected from the group consisting of SEQ ID Nos: 1, 3, 5, 7, 9, 11, 13, 15, 17, 19, 21, 23, 25, 27, 29, 31, complements thereof, fragments thereof, and sequences at least 70% identical thereto, wherein said sequences encode a functional FIT2 protein. In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 1. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 3. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 5. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 7. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 9. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 11. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 13. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 15. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 17. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 19. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 21. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 23. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 25. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 27. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 29. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 31. In some embodiments, the disclosure relates to genetic constructs comprising these sequences.

The disclosure also encompasses variants and fragments of proteins of an amino acid sequence encoded by the nucleic acid sequences of FIT2, orthologs of FIT2 and/or paralogs of FIT2. The variants may contain alterations in the amino acid sequences of the constituent proteins. The term “variant” with respect to a polypeptide refers to an amino acid sequence that is altered by one or more amino acids with respect to a reference sequence. The variant can have “conservative” changes, or “nonconservative” changes, e.g., analogous minor variations can also include amino acid deletions or insertions, or both.

Functional fragments and variants of a polypeptide include those fragments and variants that maintain one or more functions or domains of the parent polypeptide. As used herein, a protein domain is a distinct functional and/or structural unit in a protein, and are usually responsible for a particular function or interaction. It is recognized that the gene or cDNA encoding a polypeptide can be considerably mutated without materially altering one or more of the polypeptide's functions and/or domains. First, the genetic code is well-known to be degenerate, and thus different codons encode the same amino acids. Second, even where an amino acid substitution is introduced, the mutation can be conservative and have no material impact on the essential function(s) of a protein. See, e.g., Stryer Biochemistry 3^{rd} Ed., 1988. Third, part of a polypeptide chain can be deleted without impairing or eliminating all of its functions. Fourth, insertions or additions can be made in the polypeptide chain for example, adding epitope tags, without impairing or eliminating its functions (Ausubel et al. J. Immunol. 159(5): 2502-12, 1997). Other modifications that can be made without materially impairing one or more functions of a polypeptide can include, for example, in vivo or in vitro chemical and biochemical modifications or the incorporation of unusual amino acids. Such modifications include, but are not limited to, for example, acetylation, carboxylation, phosphorylation, glycosylation, ubiquination, labelling, e.g., with radionucleotides, and various enzymatic modifications, as will be readily appreciated by those well skilled in the art. A variety of methods for labelling polypeptides, and labels useful for such purposes, are well known in the art, and include radioactive isotopes such as ³²P, ligands which bind to or are bound by labelled specific binding partners (e.g., antibodies), fluorophores, chemiluminescent agents, enzymes, and anti-ligands. Functional fragments and variants can be of varying length. For example, some fragments have at least 10, 25, 50, 75, 100, 200, or even more amino acid residues. These mutations can be natural or purposely changed. In some embodiments, mutations containing alterations that produce silent substitutions, additions, or deletions, but do not alter the properties or activities of the proteins or how the proteins are made are an embodiment of the present disclosure.

Conservative amino acid substitutions are those substitutions that, when made, least interfere with the properties of the original protein, that is, the structure and especially the function of the protein is conserved and not significantly changed by such substitutions. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Further information about conservative substitutions can be found, for instance, in Ben Bassat et al. (J. Bacteriol., 169:751-757, 1987), O'Regan et al. (Gene, 77:237-251, 1989), Sahin-Toth et al. (Protein Sci., 3:240-247, 1994), Hochuli et al. (Bio/Technology, 6:1321-1325, 1988) and in widely used textbooks of genetics and molecular biology. The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table 1 shows exemplary conservative amino acid substitutions.

TABLE 1

| | | Highly Conserved | Conserved |
|---|---|---|---|
| | Very Highly - | Substitutions (from | Substitutions |
| Original | Conserved | the Blosum90 | (from the Blosum65 |
| Residue | Substitutions | Matrix) | Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, | Arg, Asp, Gln, Glu, His, |
| | | Ser, Thr | Lys, Ser, Thr |
| Asp | Glu | Asn, Glu | Asn, Gln, Glu, Ser |
| Cys | Ser | None | Ala |
| Gln | Asn | Arg, Asn, Glu, His, | Arg, Asn, Asp, Glu, His, |
| | | Lys, Met | Lys, Met, Ser |
| Glu | Asp | Asp, Gln, Lys | Arg, Asn, Asp, Gln, His, |
| | | | Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, |
| | | | Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

### Codon Optimization

In some cases, variants may differ from the disclosed sequences by alteration of the coding region to fit the codon usage bias of the particular organism into which the molecule is to be introduced. In other cases, the coding region may be altered by taking advantage of the degeneracy of the genetic code to alter the coding sequence such that, while the nucleotide sequence is substantially altered, it nevertheless encodes a protein having an amino acid sequence substantially similar to the disclosed amino acid sequences of FIT2, orthologs of FIT2 and/or paralogs of FIT2, and/or fragments and variations thereof.

Protein expression is governed by a host of factors including those that affect transcription, mRNA processing, and stability and initiation of translation. Optimization can thus address any of a number of sequence features of any particular gene. Translation may be paused due to the presence of codons in the polynucleotide of interest that are rarely used in the host organism, and this may have a negative effect on protein translation due to their scarcity in the available tRNA pool. Specifically, it can result in reduced protein expression.

Alternate translational initiation also can result in reduced heterologous protein expression. Alternate translational initiation can include a synthetic polynucleotide sequence inadvertently containing motifs capable of functioning as a ribosome binding site (RBS). These sites can result in initiating translation of a truncated protein from a gene-internal site. One method of reducing the possibility of producing a truncated protein includes eliminating putative internal RBS sequences from an optimized polynucleotide sequence.

Repeat-induced polymerase slippage can result in reduced heterologous protein expression. Repeat-induced polymerase slippage involves nucleotide sequence repeats that have been shown to cause slippage or stuttering of DNA polymerase which can result in frameshift mutations. Such repeats can also cause slippage of RNA polymerase. In an organism with a high G+C content bias, there can be a higher degree of repeats composed of G or C nucleotide repeats. Therefore, one method of reducing the possibility of inducing RNA polymerase slippage, includes altering extended repeats of G or C nucleotides.

Interfering secondary structures also can result in reduced heterologous protein expression. Secondary structures can sequester the RBS sequence or initiation codon and have been correlated to a reduction in protein expression. Stemloop structures can also be involved in transcriptional pausing and attenuation. An optimized polynucleotide sequence can contain minimal secondary structures in the RBS and gene coding regions of the nucleotide sequence to allow for improved transcription and translation.

The optimization process can begin, for example, by identifying the desired amino acid sequence to be expressed by the host. From the amino acid sequence, a candidate polynucleotide or DNA sequence can be designed. During the design of the synthetic DNA sequence, the frequency of codon usage can be compared to the codon usage of the host expression organism and rare host codons can be removed from the synthetic sequence. Additionally, the synthetic candidate DNA sequence can be modified in order to remove undesirable enzyme restriction sites and add or remove any desired signal sequences, linkers or untranslated regions. The synthetic DNA sequence can be analyzed for the presence of secondary structure that may interfere with the translation process, such as G/C repeats and stem-loop structures.

Optimized coding sequences containing codons preferred by a particular host can be prepared, for example, to increase the rate of translation or to produce recombinant RNA transcripts having desirable properties, such as a longer half-life, as compared with transcripts produced from a non-optimized sequence.

### Functional Fragments, Chimeric, and Synthetic Polypeptides

In some cases, functional fragments derived from FIT2 orthologs of the present disclosure can still confer resistance to pathogens when expressed in a plant. In some cases, the functional fragments contain one or more conserved regions shared by two or more FIT2 orthologs.

In some cases, functional chimeric or synthetic polypeptides derived from the FIT2 orthologs of the present disclosure are provided. The functional chimeric or synthetic polypeptides can still confer resistance to pathogens when expressed in a plant. In some cases, the functional chimeric or synthetic polypeptides contain one or more conserved regions shared by two or more FIT2 orthologs.

### DNA Constructs

In some embodiments, the disclosure relates to a DNA construct comprising at least one FIT2 sequence disclosed herein. In some cases, the FIT2 sequence is a polynucleotide comprising a nucleic acid sequence encoding a FIT2 protein, wherein the protein is selected from the group consisting of: SEQ ID Nos: 2, 4, 6, 8, 10, 12, 14, 16, 18, 20, 22, 24, 26, 28, 30, 32, functional homologs, and/or fragments and variations thereof. In some cases, the FIT2 protein shares at least about 70% identity to SEQ ID NO: 2. In some cases, the FIT2 sequence is selected from SEQ ID Nos: 1, 3, 5, 7, 9, 11, 13, 15, 17, 19, 21, 23, 25, 27, 29, 31, complements thereof, fragments thereof, and sequences at least 70% identical thereto. In some cases, two or more FIT2 sequences are stacked to increase pathogen resistance in a plant. In some cases, at least one FIT2 sequence is stacked with another pathogen resistance gene. In some cases, at least one FIT2 sequence is stacked with a TIR-NLR signaling gene.

The expression control elements used to regulate the expression of a given protein can either be the expression control element that is normally found associated with the coding sequence (homologous expression element) or can be a heterologous expression control element. A variety of homologous and heterologous expression control elements are known in the art and can readily be used to make DNA constructs for use in the present disclosure. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 19S and 35S promoters (CaMV 19S and CaMV 35S promoters, respectively) to control gene expression in a plant (U.S. Pat. Nos. 5,352,605; 5,530,196 and 5,858,742 for example). Enhancer sequences derived from the CaMV can also be utilized (U.S. Pat. Nos. 5,164,316; 5,196,525; 5,322,938; 5,530,196; 5,352,605; 5,359,142; and 5,858,742 for example). Lastly, plant promoters such as prolifera promoter, fruit specific promoters, Ap3 promoter, heat shock promoters, seed specific promoters, etc. can also be used.

Either a gamete-specific promoter, a constitutive promoter (such as the CaMV or Nos promoter), an organ-specific promoter (such as the E8 promoter from tomato), or an inducible promoter is typically ligated to the protein or antisense encoding region using standard techniques known in the art. The DNA construct may be further optimized by employing supplemental elements such as transcription terminators and/or enhancer elements.

Thus, for expression in plants, the DNA construct will typically contain, in addition to the protein sequence, a plant promoter region, a transcription initiation site and a transcription termination sequence. Unique restriction enzyme sites at the 5′ and 3′ ends of the expression unit are typically included to allow for easy insertion into a pre-existing vector.

In the construction of heterologous promoter/gene of interest or antisense combinations, the promoter is preferably positioned about the same distance from the heterologous transcription start site as it is from the transcription start site in its natural setting. As is known in the art, however, some variation in this distance can be accommodated without loss of promoter function.

In addition to a promoter sequence, the expression cassette can also contain a transcription termination region downstream of the gene to provide for efficient termination.

The termination region may be obtained from the same gene as the promoter sequence or may be obtained from different genes. If the mRNA encoded by the gene is to be efficiently processed, DNA sequences which direct polyadenylation of the RNA are also commonly added to the vector construct. Polyadenylation sequences include, but are not limited to the Agrobacterium octopine synthase signal (Gielen et al., EMBO J 3:835-846 (1984)) or the nopaline synthase signal (Depicker et al., Mol. and Appl. Genet. 1:561-573 (1982)). The resulting expression unit is ligated into or otherwise constructed to be included in a vector that is appropriate for higher plant transformation. One or more expression units may be included in the same vector.

Given that AvrFIT2 is highly conserved in all fungal species, FIT2 may be able to detect AvrFIT2 from beneficial mycorrhizal fungi that form symbioses with plant roots and help with nutrient and water uptake. Expression of FIT2 in root tissue may perturb associations with these fungi, in particular that with endomycorrhizal fungi that penetrate root tissue and form arbuscules. The amount of perturbation will depend on the specific FIT2 and AvrFIT2 alleles present, the amount of AvrFIT2 delivered into the plant cell, the specific mechanisms the fungi use to supress plant immunity, and the amount of FIT2 expression in the colonized tissue. Use of a promoter to drive FIT2 expression that minimizes or avoids expression of FIT2 in tissue colonized by beneficial mycorrhizal fungi can avoid inhibition of this symbiosis. Alternatively, an allele of FIT2 can be selected that is not active or is minimally active against desired mycorrhizal species. Such an allele can be readily selected using the described transient activity assay.

### Selection

A DNA construct will typically contain a selectable marker gene expression unit by which transformed plant cells can be identified in culture. Usually, the marker gene will encode resistance to an antibiotic, such as G418, hygromycin, bleomycin, kanamycin, or gentamicin or to an herbicide, such as glyphosate (Round-Up) or glufosinate (BASTA) or atrazine. Replication sequences, of bacterial or viral origin, are generally also included to allow the vector to be cloned in a bacterial or phage host; preferably a broad host range for prokaryotic origin of replication is included. A selectable marker for bacteria may also be included to allow selection of bacterial cells bearing the desired construct. Suitable prokaryotic selectable markers include resistance to antibiotics such as ampicillin, kanamycin or tetracycline. Other DNA sequences encoding additional functions may also be present in the vector, as is known in the art. For instance, in the case of Agrobacterium transformations, T-DNA sequences will also be included for subsequent transfer to plant chromosomes.

For positive selection, for example, a foreign gene is supplied to a plant cell that allows it to utilize a substrate present in the medium that it otherwise could not use, such as mannose or xylose (for example, refer U.S. Pat. Nos. 5,767,378; 5,994,629). More typically, however, negative selection is used because it is more efficient, utilizing selective agents such as herbicides or antibiotics that either kill or inhibit the growth of non-transformed plant cells and reducing the possibility of chimeras. Resistance genes that are effective against negative selective agents are provided on the introduced foreign DNA used for the plant transformation. For example, one of the most popular selective agents used is the antibiotic kanamycin, together with the resistance gene neomycin phosphotransferase (nptII), which confers resistance to kanamycin and related antibiotics (see, for example, Messing & Vierra, Gene 19: 259-268 (1982); Bevan et al., Nature 304:184-187 (1983)). However, many different antibiotics and antibiotic resistance genes can be used for transformation purposes (refer U.S. Pat. Nos. 5,034,322, 6,174,724 and 6,255,560). In addition, several herbicides and herbicide resistance genes have been used for transformation purposes, including the bar gene, which confers resistance to the herbicide phosphinothricin (White et al., Nucl Acids Res 18: 1062 (1990), Spencer et al., Theor Appl Genet 79: 625-631(1990), U.S. Pat. Nos. 4,795,855, 5,378,824 and 6,107,549). In addition, the dhfr gene, which confers resistance to the anticancer agent methotrexate, has been used for selection (Bourouis et al., EMBO J. 2(7): 1099-1104 (1983).

### Transgenic Plants Comprising Sequences Disclosed Herein

In one embodiment, the present disclosure relates to a transgenic plant, plant part, or plant cell, wherein the transgene comprises at least one polynucleotide coding for FIT2, orthologs of FIT2 and/or paralogs of FIT2, and/or fragments and variations thereof, and exhibit resistance or tolerance to a pathogen. In some cases, the polynucleotide encodes a protein selected from the group consisting of: SEQ ID NOs: 2, 4, 6, 8, 10, 12, 14, 16, 18, 20, 22, 24, 26, 28, 30, 32, proteins at least 90% identical thereto, and functional homologs thereof. In some cases, the polynucleotide comprises a nucleic acid sequence selected from the group consisting of SEQ ID NOs: 1, 3, 5, 7, 9, 11, 13, 15, 17, 19, 21, 23, 25, 27, 29, 31, complements thereof, fragments thereof, and sequences at least 70% identical thereto. In some cases, the pathogen is a fungus. In some cases, the fungus is from the order Cantharellales, Mucorales, Ustilaginales, Atheliales, or Pucciniales. In some cases, the fungal pathogen is a Basidiomycota, Mucoromycota, Ascomycota, or Oomycota. In some cases, the fungal pathogen is Choanephora cucurbitarum, Ustilago maydis, Athelia spp., Rhizoctonia solani, Melampsora spp., Phakopsora pachyrhizi, Phakopsora meibomiae, Phakopsora euvitis, Phakopsora spp., Puccinia spp., Uromyces spp., Austropuccinia spp., Cronartium spp. Hemileia vastatri, or is a pathogen that produces an AvrFIT2 protein selected from the list shown in Table 2 below.

TABLE 2
Example AvrFIT2 alleles from diverse pathogens

| Species | Accession No. |
|---|---|
| Rhizoctonia solani | KEP52120.1 |
| Fusarium oxysporum | XP_018235786.1 |
| Athelia sp. | KAF7985119.1 |
| Corynespora cassiicola | JAEMHD010000003.1 |
| Colletotrichum sojae | KAF6821048.1 |
| Macrophomina phaseolina | EKG16650.1 |
| Sclerotinia sclerotiorum | XP_001591874.1 |
| Ustilago maydis | XP_011392666.1 |
| Alternaria alternata | XP_018390749.1 |
| Hemileia vastatrix | PZQQ01059748.1 |
| Austropuccinia psidii | MBW0502619.1 |
| Cronartium quercuum | KAG0139904.1 |
| Cercospora berteroae | 8982 |
| Verticillium alfalfae | XP_003006500.1 |
| Choanephora cucurbitarum | OBZ88984.1 |
| Blumeria graminis | EPQ67290.1 |
| Phytophthora sojae | XP_009519860.1 |
| Uromyces viciae-fabae | KN362125.1 |
| Albugo laibachii | AlNc14C15G1709 |
| Fulvia fulva | UJO12982.1 |
| Puccinia striiformis | KNE93671.1 |
| Puccinia triticina | OAV98102.1 |
| Puccinia graminis | XP_003319246.1 |
| Pythium brassicum | TYZ60240.1 |
| Puccinia sorghi | VP01_2579g2 |
| Peronospora effusa | DD238 003577 |
| Phakopsora pachyrhizi | ALL41303.1 |
| Melampsora larici-populina | XP_007410633.1 |

In some cases, the plant, plant part, or plant cell is in the subfamily Papilionoideae. In some cases, the plant, plant part, or plant cell is Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

In some cases, the plant, plant part, or plant cell is Glycine max, and the plant, plant part, or plant cell is resistant to Asian Soybean Rust caused by Phakopsora pachyrhizi.

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety.

Microprojectile bombardment is also known as particle acceleration, biolistic bombardment, and the gene gun (Biolistic® Gene Gun). The gene gun is used to shoot pellets that are coated with genes (e.g., for desired traits) into plant seeds or plant tissues in order to get the plant cells to then express the new genes. The gene gun uses an actual explosive (.22 caliber blank) to propel the material. Compressed air or steam may also be used as the propellant. The Biolistic® Gene Gun was invented in 1983-1984 at Cornell University by John Sanford, Edward Wolf, and Nelson Allen. It and its registered trademark are now owned by E. I. du Pont de Nemours and Company. Most species of plants can be been transformed using this method.

The most common method for the introduction of new genetic material into a plant genome involves the use of living cells of the bacterial pathogen Agrobacterium tumefaciens. Agrobacterium tumefaciens is a naturally occurring bacterium that is capable of inserting its DNA (genetic information) into plants, resulting in a type of injury to the plant known as crown gall. Most species of plants can now be transformed using this method. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1. Methods of Agrobacterium-mediated plant transformation that involve using vectors with no T-DNA are also well known to those skilled in the art and can be used with the methods of the present disclosure. See, for example, U.S. Pat. No. 7,250,554, which utilizes P-DNA instead of T-DNA in the transformation vector. A transgenic plant formed using Agrobacterium transformation methods typically contains a single gene on one chromosome, although multiple copies are possible. Such transgenic plants can be referred to as being hemizygous for the added gene, or may be referred to as an independent segregant, because each transformed plant represents a unique T-DNA integration event (U.S. Pat. No. 6,156,953).

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988).

Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope (U.S. Pat. Nos. 5,204,253, 5,015,580).

A third direct method uses fibrous forms of metal or ceramic consisting of sharp, porous or hollow needle-like projections that impale the cells, and also the nuclear envelope of cells. Both silicon carbide and aluminium borate whiskers have been used for plant transformation (Mizuno et al., 2004; Petolino et al., 2000; U.S. Pat. No. 5,302,523 US Application 20040197909) and also for bacterial and animal transformation (Kaepler et al., 1992; Raloff, 1990; Wang, 1995).

Examples of viral vectors include, but are not limited to, recombinant plant viruses. Non-limiting examples of plant viruses include, TMV-mediated (transient) transfection into tobacco (Tuipe, T-H et al (1993), J. Virology Meth, 42: 227-239), ssDNA genomes viruses (e.g., family Geminiviridae), reverse transcribing viruses (e.g., families Caulimoviridae, Pseudoviridae, and Metaviridae), dsNRA viruses (e.g., families Reoviridae and Partitiviridae), (−) ssRNA viruses (e.g., families Rhabdoviridae and Bunyaviridae), (+) ssRNA viruses (e.g., families Bromoviridae, Closteroviridae, Comoviridae, Luteoviridae, Potyviridae, Sequiviridae and Tombusviridae) and viroids (e.g., families Pospiviroldae and Avsunviroidae). Detailed classification information of plant viruses can be found in Fauquet et al (2008, “Geminivirus strain demarcation and nomenclature”. Archives of Virology 153:783-821, incorporated herein by reference in its entirety), and Khan et al. (Plant viruses as molecular pathogens; Publisher Routledge, 2002, ISBN 1560228954, 9781560228950). Examples of non-viral vectors include, but are not limited to, liposomes, polyamine derivatives of DNA, and the like.

Non-limiting examples of binary vectors suitable for soybean species transformation and transformation methods are described by Yi et al. 2006 (Transformation of multiple soybean cultivars by infecting cotyledonary-node with Agrobacterium tumefaciens, African Journal of Biotechnology Vol. 5 (20), pp. 1989-1993, 16 Oct. 2006), Paz et al., 2004 (Assessment of conditions affecting Agrobacterium-mediated soybean transformation using the cotyledonary node explant, Euphytica 136: 167-179, 2004), U.S. Pat. Nos. 5,376,543, 5,416,011, 5,968,830, and 5,569,834, or by similar experimental procedures well known to those skilled in the art.

Genes can also be introduced in a site directed fashion using homologous recombination. Homologous recombination permits site-specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

### Genetically Engineering a Pathogen Resistance or Tolerance Trait in a Plant, Plant Part, or Plant Cell

An embodiment of the present disclosure teaches a method of genetically engineering a pathogen resistance or tolerance trait in a plant, plant part, or plant cell, comprising: providing a plant species that is susceptible to a pathogen; identifying within the genome of the plant species an endogenous homolog of FIT2, wherein said endogenous homolog is nonfunctional (does not mediate AvrFIT2 recognition), and/or is not expressed at levels high enough to convey resistance, and genetically modifying a plant, plant part, or plant cell from the susceptible plant species with targeted gene editing, wherein said targeted gene editing is directed towards the nonfunctional or poorly expressed endogenous FIT2 homolog, and wherein said targeted gene editing restores the function of the endogenous FIT2 by enabling the FIT2 homolog to recognize AvrFIT2 and/or by altering the expression level or expression pattern of the endogenous FIT2 homolog. In some embodiments, the targeted gene editing is inserting a promoter. Examples of plant promoters that can be used to alter the expression of a gene are well known in the art and examples are discussed herein. In some embodiments, the targeted gene editing is inserting the endogenous FIT2 gene into a non-essential native gene to increase expression of the endogenous FIT2 gene.

In some embodiments, expression of the endogenous FIT2 homolog in the genetically engineered plant, plant part, or plant cell is increased at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold compared to a non-genetically engineered plant, plant part, or plant cell.

In some embodiments, the genetically engineered plant, plant part, or plant cell is a species of Glycine spp., and the endogenous FIT2 gene encodes a protein at least 90% identical to SEQ ID NO: 16, at least 91% identical to SEQ ID NO: 16, at least 92% identical to SEQ ID NO: 16, at least 93% identical to SEQ ID NO: 16, at least 94% identical to SEQ ID NO: 16, at least 95% identical to SEQ ID NO: 16, at least 96% identical to SEQ ID NO: 16, at least 97% identical to SEQ ID NO: 16, at least 98% identical to SEQ ID NO: 16, or at least 99% identical to SEQ ID NO: 16.

As used herein, a “nonfunctional” FIT2 homolog is a homolog that does not recognize a pathogen protein homolog of AvrFIT2. FIT2 homologs may identified by any number of means known in the art. Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Nat. Acad. Sci., 85:2444, 1988); Higgins and Sharp (Gene, 73:237-44, 1988); Higgins and Sharp (CABIOS, 5:151-53, 1989); Corpet et al. (Nuc. Acids Res., 16:10881-90, 1988); Huang et al. (Comp. Appls Biosci., 8:155-65, 1992); and Pearson et al. (Meth. Mol. Biol., 24:307-31, 1994). Altschul et al. (Nature Genet., 6:119-29, 1994) presents a detailed consideration of sequence alignment methods and homology calculations.

Restoring the function of a FIT2 homolog as used herein relates to modifying the allele such that it restores the recognition of a pathogen protein such as AvrFIT2 and/or restores expression level to confer resistance or tolerance to a pathogen. Restoring the function of a homologous gene by way of genetic engineering has been done and is well known in the art (see for example, Ivics Z, et al., (1997), “Molecular reconstruction of Sleeping Beauty, a Tcl-like transposon from fish, and its transposition in human cells”, Cell. 91 (4): 501-510, and recently, Suh S, et al., Restoration of visual function in adult mice with an inherited retinal disease via adenine base editing, Nat Bioned Eng (2020) and Sedeek K, et aL, Plant genome engineering for targeted improvement of crop traits, Front. Plant Sci., 12 Feb. 2019).

In some embodiments, the targeted gene editing uses an engineered or natural nuclease selected from the group consisting of homing endonucleases/meganucleases (EMNs), zinc finger nucleases (ZFNs), and transcription activator-like effector nucleases (TALENs). In some embodiments, the targeted gene editing uses a clustered regularly interspaced short palindromic repeats (CRISPR)-Cas nuclease. In some embodiments, the nuclease is selected from the group consisting of Cas9, Cas12, Cas13, CasX, and CasY. The disclosure also relates to plants, plant parts, and plant cells exhibiting resistance or tolerance to a pathogen produced by genetic modification of a FIT2 homolog.

### Gene Editing Using CRISPR

Targeted gene editing can be done using CRISPR technology (Saunders & Joung, Nature Biotechnology, 32, 347-355, 2014). CRISPR is a type of genome editing system that stands for Clustered Regularly Interspaced Short Palindromic Repeats. This system and CRISPR-associated (Cas) genes enable organisms, such as select bacteria and archaea, to respond to and eliminate invading genetic material. Ishino, Y., et al. J. Bacteriol. 169, 5429-5433 (1987). These repeats were known as early as the 1980s in E. coli, but Barrangou and colleagues demonstrated that S. thermophilus can acquire resistance against a bacteriophage by integrating a fragment of a genome of an infectious virus into its CRISPR locus. Barrangou, R., et al. Science 315, 1709-1712 (2007). Many plants have already been modified using the CRISPR system, including soybean (see for example, Han J, et al., Creation of early flowering germplasm of soybean by CRISPR/Cas9 Technology, Front. Plant Sci., 22 Nov. 2019), and many Cas genes have now been characterized and used with the system (see for example, Wang J, et al., The rapidly advancing Class 2 CRISPR-Cas technologies: A customizable toolbox for molecular manipulations. J Cell Mol Med. 2020; 24(6):3256-3270).

Gene editing can also be done using crRNA-guided surveillance systems for gene editing. Additional information about crRNA-guided surveillance complex systems for gene editing can be found in the following documents, which are incorporated by reference in their entirety: U.S. Application Publication No. 2010/0076057 (Sontheimer et al., Target DNA Interference with crRNA); U.S. Application Publication No. 2014/0179006 (Feng, CRISPR-CAS Component Systems, Methods, and Compositions for Sequence Manipulation); U.S. Application Publication No. 2014/0294773 (Brouns et al., Modified Cascade Ribonucleoproteins and Uses Thereof); Sorek et al., Annu. Rev. Biochem. 82:237-266, 2013; and Wang, S. et al., Plant Cell Rep (2015) 34: 1473-1476.

### Gene Editing Using TALENs

Transcription activator-like effector nucleases (TALENs) have been successfully used to introduce targeted mutations via repair of double stranded breaks (DSBs) either through non-homologous end joining (NHEJ), or by homology-directed repair (HDR) and homology-independent repair in the presence of a donor template. Thus, TALENs are another mechanism for targeted genome editing in plants. The technique is well known in the art; see for example Malzahn, Aimee et al. “Plant genome editing with TALEN and CRISPR” Cell & Bioscience vol. 7 21. 24 Apr. 2017.

### Other Methods of Genome Editing

In addition to CRISPR and TALENs, two other types of engineered nucleases can be used for genome editing: engineered homing endonucleases/meganucleases (EMNs), and zinc finger nucleases (ZFNs). These methods are well known in the art. See for example, Petilino, Joseph F. “Genome editing in plants via designed zinc finger nucleases” In Vitro Cell Dev Biol Plant. 51(1): pp. 1-8 (2015); and Daboussi, Fayza, et al. “Engineering Meganuclease for Precise Plant Genome Modification” in Advances in New Technology for Targeted Modification of Plant Genomes. Springer Science+Business. pp 21-38 (2015).

### Breeding Methods

Once a gene has been introduced into a plant, or a gene has been genetically modified, that plant can then be used in conventional plant breeding schemes (e.g., pedigree breeding, single-seed-descent breeding schemes, recurrent selection, backcross breeding) to produce progeny which also contain the gene or modified trait. Thus, another aspect of the present disclosure relates to breeding with, or asexually propagating, plants having been transformed with a FIT2 homolog or an immune receptor gene coding for a protein that recognizes AvrFIT2, or plants wherein a FIT2 nonfunctional homolog was genetically modified to restore function, wherein the plants exhibit resistance or tolerance to a pathogen. The disclosure further relates to progeny plants produced therefrom.

In some cases, plants or progeny therefrom comprising the gene or modified trait may further comprise one or more additional desired traits. In some cases, the one or more additional desired traits are stacked on the same construct as the gene (for example, the FIT2 genes disclosed herein). In another case, the one or more additional desired traits may be introgressed by conventional breeding.

### Backcross Breeding

Backcross breeding has been used to transfer genes for a simply inherited, highly heritable trait into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent. As used herein, backcross breeding is synonymous with introgression. Plants produced therefrom may be referred to a single locus converted or single gene converted plants.

A non-limiting example of a backcross breeding protocol would be the following: a) the first generation F₁ produced by the cross of the recurrent parent A by the donor parent B is backcrossed to parent A, b) selection is practiced for the plants having the desired trait of parent B, c) selected plants are self-pollinated to produce a population of plants where selection is practiced for the plants having the desired trait of parent B and physiological and morphological characteristics of parent A, d) the selected plants are backcrossed one, two, three, four, five, six, seven, eight, nine, or more times to parent A to produce selected backcross progeny plants comprising the desired trait of parent B and the physiological and morphological characteristics of parent A. Step (c) may or may not be repeated and included between the backcrosses of step (d).

Examples of desired traits include, but are not limited to, herbicide resistance (such as bar or pat genes), resistance for bacterial, fungal, or viral disease (such as gene I used for BCMV resistance), insect resistance, enhanced nutritional quality (such as 2s albumin gene), industrial usage, agronomic qualities (such as the “persistent green gene”), yield stability, and yield enhancement.

### Pedigree Selection

Pedigree breeding is used commonly for the improvement of self-pollinating crops or inbred lines of cross-pollinating crops. Two parents possessing favorable, complementary traits are crossed to produce an F₁. An F₂ population is produced by selfing one or several F_{1S} or by intercrossing two F₁ₛ (sib mating). The dihaploid breeding method could also be used. Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release of new cultivars. Similarly, the development of new cultivars through the dihaploid system requires the selection of the cultivars followed by two to five years of testing in replicated plots.

### Open-Pollination

The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988). For population improvement methods specific for soybean see, e.g., J.R. Wilcox, editor (1987) SOYBEANS: Improvement, Production, and Uses, Second Edition, American Society of Agronomy, Inc., Crop Science Society of America, Inc., and Soil Science Society of America, Inc., publishers, 888 pages.

### Hand-Pollination Method

Hand pollination describes the crossing of plants via the deliberate fertilization of female ovules with pollen from a desired male parent plant. In some cases the donor or recipient female parent and the donor or recipient male parent line are planted in the same field or in the same greenhouse. The inbred male parent can be planted earlier than the female parent to ensure adequate pollen supply at the pollination time. Pollination is started when the female parent flower is ready to be fertilized. Female flower buds that are ready to open in the following days are identified, covered with paper cups or small paper bags that prevent bee or any other insect from visiting the female flowers, and marked with any kind of material that can be easily seen the next morning. The male flowers of the male parent are collected in the early morning before they are open and visited by pollinating insects. The covered female flowers of the female parent, which have opened, are un-covered and pollinated with the collected fresh male flowers of the male parent, starting as soon as the male flower sheds pollen. The pollinated female flowers are again covered after pollination to prevent bees and any other insects visit. The pollinated female flowers are also marked. The marked flowers are harvested. In some cases, the male pollen used for fertilization has been previously collected and stored.

### Bee-Pollination Method

Using the bee-pollination method, the parent plants are usually planted within close proximity. More female plants may be planted to allow for a greater production of seed. Insects are placed in the field or greenhouses for transfer of pollen from the male parent to the female flowers of the female parent.

### Mass Selection

In mass selection, desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

### Synthetics

A synthetic variety is produced by crossing inter se a number of genotypes selected for good combining ability in all possible hybrid combinations, with subsequent maintenance of the variety by open pollination. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

### Hybrids

A hybrid is an individual plant resulting from a cross between parents of differing genotypes. Commercial hybrids are now used extensively in many crops, including corn (maize), sorghum, sugar beet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

### Bulk Segregation Analysis (BSA)

BSA, a.k.a. bulked segregation analysis, or bulk segregant analysis, is a method described by Michelmore et al. (Michelmore et al., 1991, Identification of markers linked to disease-resistance genes by bulked segregant analysis: a rapid method to detect markers in specific genomic regions by using segregating populations. Proceedings of the National Academy of Sciences, USA, 99:9828-9832) and Quarrie et al. (Quarrie et al., Bulk segregant analysis with molecular markers and its use for improving drought resistance in maize, 1999, Journal of Experimental Botany, 50(337):1299-1306).

For BSA of a trait of interest, parental lines with certain different phenotypes are chosen and crossed to generate F₂, doubled haploid or recombinant inbred populations with QTL analysis. The population is then phenotyped to identify individual plants or lines having high or low expression of the trait. Two DNA bulks are prepared, one from the individuals having one phenotype (e.g., resistant to pathogen), and the other from the individuals having reversed phenotype (e.g., susceptible to pathogen), and analyzed for allele frequency with molecular markers. Only a few individuals are required in each bulk (e.g., 10 plants each) if the markers are dominant (e.g., RAPDs). More individuals are needed when markers are co-dominant (e.g., RFLPs). Markers linked to the phenotype can be identified and used for breeding or QTL mapping.

### Gene Pyramiding

The method to combine into a single genotype a series of target genes identified in different parents is usually referred as gene pyramiding. The first part of a gene pyramiding breeding is called a pedigree and is aimed at cumulating one copy of all target genes in a single genotype (called root genotype). The second part is called the fixation steps and is aimed at fixing the target genes into a homozygous state, that is, to derive the ideal genotype (ideotype) from the root genotype. Gene pyramiding can be combined with marker assisted selection (MAS, see Hospital et al., 1992, 1997a, and 1997b, and Moreau et al, 1998) or marker based recurrent selection (MBRS, see Hospital et al., 2000).

Examples of Additional Desired Traits that May be Stacked with the Pathogen Resistance or Tolerance Traits Disclosed Herein

In some cases, multiple FIT genes may be combined in a single plant to increase and/or broaden pathogen resistance, for example FIT1 and FIT2. In some cases, one or more FIT1 and/or FIT2 alleles are combined with additional desired traits. These traits may be introduced to a plant through conventional breeding methods, stacked on one or more DNA constructs, and/or generated through targeted mutagenesis. Examples of additional desired traits include, but are not limited to, male sterility, herbicide resistance, resistance for bacterial, fungal, or viral disease, insect resistance, male fertility, enhanced nutritional quality, industrial usage, yield stability, and yield enhancement. Several of these traits are described in, for example, U.S. Pat. Nos. 5,959,185, 5,973,234, and 5,977,445.

In another embodiment, the disclosure teaches a method of producing a plant, plant part, or plant cell having resistance or tolerance to a pathogen wherein the method comprises transforming a plant, plant part, or plant cell with a polynucleotide encoding a functional FIT2 protein along with additional genes for TIR-NLR signaling or function, which may be missing, incompatible with FIT2, or suppressed by the pathogen in the target plant. Such genes for TIR-NLR signaling are known in the scientific literature and include, for example, EDS1, SAG101, NRG1, ADR1, and PAD4 (see also Table 3) (Wiermer et al., 2005; Ganter et al., 2019; Qi et al., 2018; Castel et al., 2019; Dong., et al 2016). Examples of pathogen effector proteins targeting and suppressing components of the plant immune system are well known (Macho and Zipfel, 2015; Zheng et al., 2018; Derevnina et al., 2021; Hulin et al., 2023). TIR-NLR signaling genes are known to be missing in several groups of plants, including most notably the absence of NRG1 and SAG101 in many monocot species (Collier et al., 2011; Ganter et al., 2019; Baggs et al., 2020). In addition to being present, the downstream signaling components must also be compatible with the immune receptor protein and other downstream components. This is not always the case for distantly related plant species. For example, EDS1 and SAG101 from Arabidopsis thaliana are not compatible with the endogenous NRG1 protein from Nicotiana benthamiana for mediating signaling from the immune receptor Roql (Lupin et al., 2019). Alleles of these downstream signaling components are readily available from the scientific literature or can be easily identified from the genome or transcriptome of a plant by BLAST search. The compatibility between different components can be tested by methods known in the art including stably or transiently knocking out the target component and complementing it by transient expression of the desired allele (Lupin et al., 2019). The alleles of SAG101, NRG1, and EDS1 from Nicotiana tabacum are compatible with FIT2, and they (or other alleles) can therefore be co-expressed with FIT2 to evade potential suppression of the native signaling components to enhance disease resistance.

TABLE 3
Example TIR-NLR signaling genes

| TIR-NLR signaling gene | Accession No. |
|---|---|
| EDS1 (Nicotiana benthamiana) | AAL85347.1 |
| SAG101 (Nicotiana tabacum) | XP_016445491.1 |
| NRG1 (Nicotiana benthamiana) | AAY54606.1 |
| ADR1 (Nicotiana tabacum) | XP_016447307.1 |
| PAD4 (Nicotiana tabacum) | XP_016443362.1 |
| EDS1 (Arabidopsis thaliana) | NP_190392.1 |
| SAG101 (Arabidopsis thaliana) | NP_568307.3 |
| NRG1 (Arabidopsis thaliana) | NP_001331503.1 |
| ADR1 (Arabidopsis thaliana) | NP_174620.2 |
| PAD4 (Arabidopsis thaliana) | NP_190811.1 |

Examples of Plant Species that May be Transformed or Modified, or Serve as a Source of Functional FIT2

The methods disclosed herein may be applied to a wide range of plants. Non-limiting examples of plants which may be transformed or modified using the methods and sequences disclosed herein include, but are not limited to, corn (Zea mays), Brassica spp. (e.g., Brassica napus, Brassica rapa, Brassica juncea), alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), broad beans (Vicia faba), tobacco (Nicotiana tabacum), potato (Solanum tuberosum), peanuts (Arachis hypogaea), cotton (Gossypium barbadense, Gossypium hirsutum), quince (Cydonia), sweet potato (Ipomoea batatas), cassava (Manihot esculenta), coffee (Coffea spp.), coconut (Cocos nucifera), pineapple (Ananas comosus), citrus trees (Citrus spp.), apple (Malus spp.), medlar (Mespilus), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), pear (Pyrus), cashew (Anacardium occidentale), macadamia (Macadamia integrifolia), almond (Prunus amygdalus), sugar beets (Beta vulgaris), sugarcane (Saccharum spp.), oats (for example Avena sativa), barley (for example Hordeum vulgare), vegetables and herbs (for example onion, leek, garlic peppermint), ornamentals (for example, Chrysanthemum, Fuchsia spp., Pelargonium, Rosa spp. Primula vulgaris), red cedar (Juniperus virginiana), and conifers (for example juniper (Juniperus communis)).

Examples of plants in the subfamily Papilionoideae include, but are not limited to, Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

Examples of legumes include, but are not limited to, the genus Phaseolus (e.g., French bean, dwarf bean, climbing bean (Phaseolus vulgaris), lima bean (Phaseolus lunatus), Tepary bean (Phaseolus acutifolius), runner bean (Phaseolus coccineus)); the genus Glycine (e.g., Glycine soja, soybeans (Glycine max (L.)); pea (Pisum) (e.g., shelling peas (sometime called smooth or round seeded peas; Pisum sativum); marrowfat pea (Pisum sativum), sugar pea (Pisum sativum), also called snow pea, edible-podded pea or mangetout, (Pisum granda)); peanut (Arachis hypogaea), clover (Trifolium spp.), medick (Medicago), kudzu vine (Pueraria lobata), common lucerne, alfalfa (Medicago sativa), chickpea (Cicer), lentils (Lens culinaris), lupins (Lupinus); vetches (Vicia), field bean, broad bean (Vicia faba), vetchling (Lathyrus) (e.g., chickling pea (Lathyrus sativus), heath pea (Lathyrus tuberosus)); genus Vigna (e.g., moth bean (Vigna aconitifolia), adzuki bean (Vigna angu laris), urd bean (Vigna mungo), mung bean (Vigna radiata), bambara groundnut (Vigna subterrane), rice bean (Vigna umbellata), Vigna vexillata, Vigna unguiculata (also known as asparagus bean, cowpea)); pigeon pea (Cajanus cajan), the genus Macrotyloma (e.g., geocarpa groundnut (Macro tyloma geocarpum), horse bean (Macrotyloma uniflorum; goa bean (Psophocarpus tetragonolobus), African yam bean (Sphenostylis stenocarpa), Egyptian black bean, lablab bean (Lablab purpureus), yam bean (Pachyrhizus erosus), guar bean (Cyamopsis tetragonolobus); and/or the genus Canavalia (e.g., jack bean (Canavalia ensiformis)), sword bean (Canavalia gladiata).

### EXAMPLES

The following examples are provided to illustrate further the various applications and are not intended to limit the disclosure beyond the limitations set forth in the appended claims.

### Example 1: FIT2 Homologs

As will be understood by one skilled in the art, homologs of FIT2 may be found in any number of species by methods described herein and methods well known in the art.

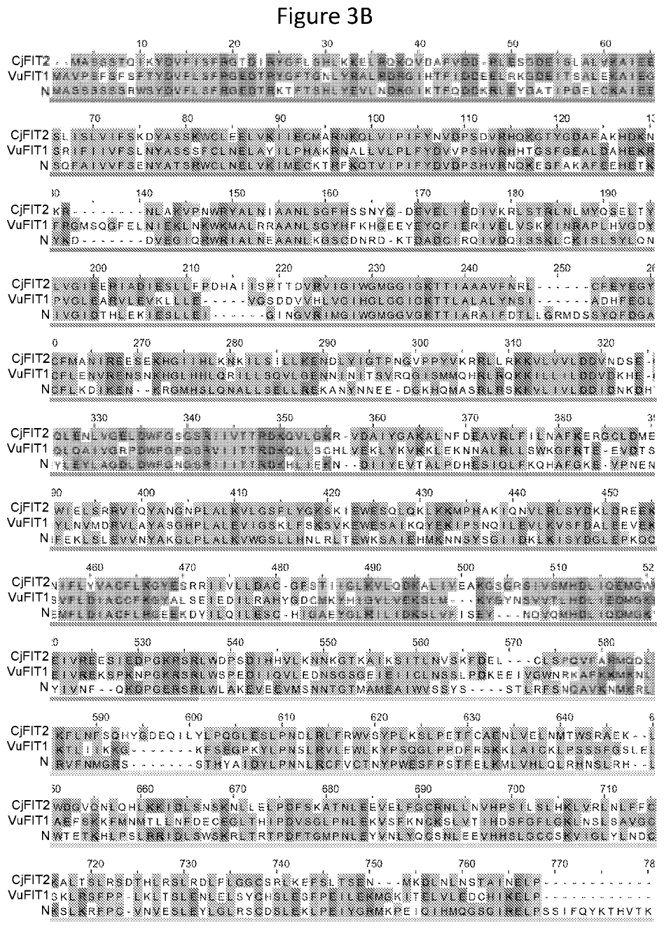

Examples of homologs of FIT2 identified are shown in FIG. 3A. FIG. 3A shows a phylogenetic tree of homologs of Crotalaria juncea FIT2 identified by performing a BLAST® search and constructing a protein alignment and phylogenetic tree of the resulting sequences. This figure shows putative FIT2 orthologs in Lupinus angustifolius, Lupinus albus, Spatholobus suberectus, Cajanus cajan, Mucuna pruriens, Glycine soja, Glycine max, Trifolium pratense, Medicago truncatula, Lotus japonicus, and Prosopis alba.

FIG. 3B shows a protein alignment of the amino acid of Crotalaria juncea FIT2 (CjFIT2) (SEQ ID NO: 2), Vigna unguiculata FIT1 (VuFIT1), and the N gene, which gives TMV resistance. VuFIT1 and N are known TIR-NLR proteins that mediate the recognition of AvrFIT1 and P50 proteins respectively. CjFIT2 is also a TIR-NLR protein but recognizes AvrFIT2, not AvrFIT1 or P50. A protein alignment shows the conservation between these proteins. The N terminal TTR domain (shown in red) functions in downstream signaling and is fairly well conserved between these proteins. The NB-ARC domain (shown in blue) binds to ATP and acts a switch between the off and on state of the receptor. It is also well conserved among these three immune receptor proteins. The C-terminal LRR domain (shown in yellow) functions in ligand binding. The LRR is poorly conserved between these proteins which is consistent with them binding to different ligands. The AvrFIT1, AvrFIT2 and P50 proteins are not related. Therefore, FIT2 is not expected to have the same activity as the N gene (which recognizes the P50 protein and confers resistance to Tobacco Mosaic Virus).

This prediction can be confirmed by transient expression of the proteins, which demonstrates that the N gene can recognize P50 but not AvrFIT2, and that FIT2 is not able to recognize P50.

### Example 2: FIT2 Expression in Glycine max

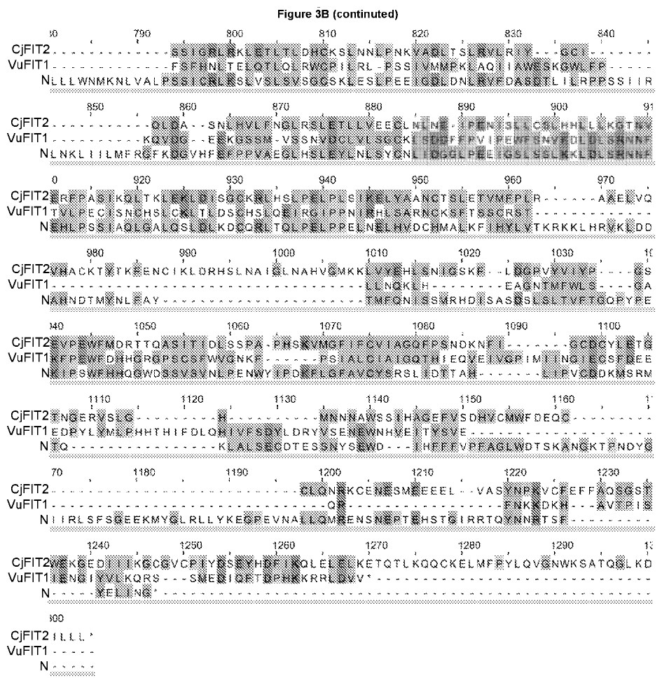

Glycine max (soybean) appears to have a functional FIT2 allele, however soybeans are susceptible to rust pathogens. To investigate this the expression of GmFIT2 relative to native soybean NLR genes was examined. FIG. 4A shows the relative transcript abundance for the top 30 NLR genes in Glycine max (soybean) and FIG. 4B shows the relative transcript abundance for the top 30 NLR genes in Crotalaria juncea. The native GmFIT2 gene is poorly expressed with an average coverage more than 30-fold lower than the average coverage of the top 30 soybean NLR genes (arrow). In contrast, CjFIT2 is well expressed in Crotalaria juncea as the 25^{th} most-expressed NLR gene. The poor expression of GmFIT2 explains why soybean is susceptible to Phakopsora pachyrhizi despite having a gene that codes for a functional FIT2 protein. The relative transcript abundance was quantified by mapping RNA-seq reads from leaf tissue to the reference transcriptome and taking the average read depth for each transcript.

### Example 3: Transient Expression of FIT2 Alleles in Leaf Tissue

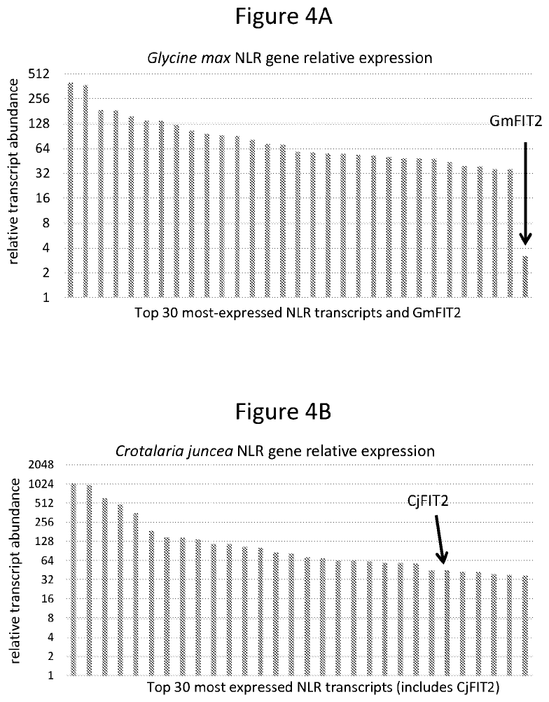

Leaf tissue from Nicotiana tabacum (lacking an endogenous FIT2) was transformed with constructs containing CjFIT2, AvrFIT2, VuFIT1, and/or AvrFITla, using standard transformation technology (an example construct comprising CjFIT2 is shown in FIG. 5). Suspensions containing the desired expression constructs were infiltrated into the leaf tissue (OD₆₀₀=0.4 total) using a needleless syringe and imaged four days post infiltration. The site of each infiltration is visible by a small punch through the leaf. Two infiltration sites (each with the same mix of the indicated proteins) were done for each combination.

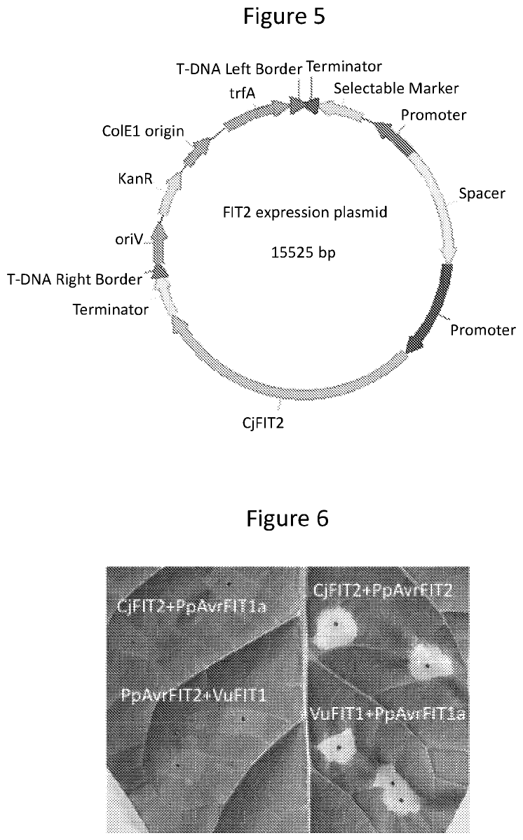

As shown in FIG. 6, co-expression of CjFIT2 and PpAvrFIT2 in Nicotiana tabacum leads to a strong cell death response, known as a hypersensitive response, that indicates strong immune activation (right side of leaf). The FIT2 gene is distinct from but in the same family as FIT1, which recognizes the AvrFIT1a/b ligand. Co-expression of VuFIT1 with PpAvrFITla also triggers a strong hypersensitive response. This response is not observed when CjFIT2 is expressed with PpAvrFITla, or when VuFIT1 is expressed with PpAvrFIT2.

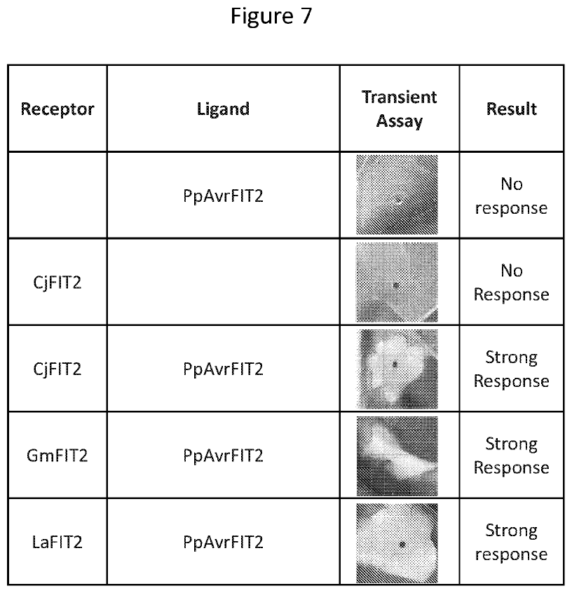

Transient expression of additional FIT2 homologs with PpAvrFIT2 are shown in FIG. 7. As shown in the top two rows of the table, expression of PpAvrFIT2 alone or CjFIT2 alone triggers no response, but co-expression of these genes triggers a strong hypersensitive cell death response indicating immune activation. FIT2 alleles from Glycine max (GmFIT2) and Lupinus albus (LaFIT2) both show the ability to recognize PpAvrFIT2. Note that despite the GmFIT2 protein being able to recognize PpAvrFIT2 in transient assays, this gene is poorly expressed in wild type soybean leaf tissue (see FIG. 4A).

These results demonstrate that FIT2 mediates the perception of AvrFIT2, and thus can confer resistance or tolerance to pathogens that express AvrFIT2.

### Example 4: CiFIT2 can Recognize Diverse AvrFIT2 Alleles

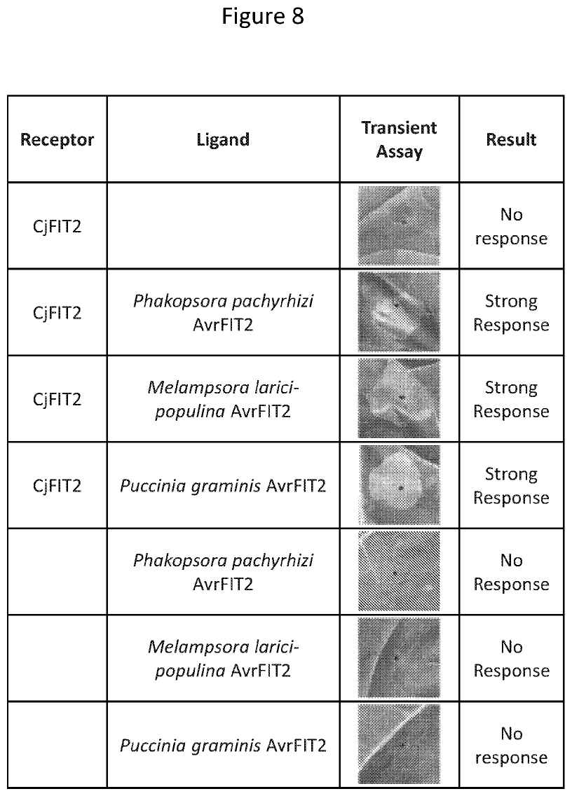

In addition to recognizing AvrFIT2 from Phakopsora pachyrhizi, the FIT2 proteins can recognize AvrFIT2 proteins from other species. CjFIT2 and AvrFIT2 alleles from Melampsora larici-populina and Puccinia graminis were expressed alone or in combination in Nicotiana tabacum leaf tissue using Agrobacterium tumefaciens. A localized hypersensitive cell death response indicates that the receptor can recognize the ligand protein. including as shown above. As shown in rows 4 and 5 of the table FIG. 8, co-expression of CjFIT2 with either Melampsora larici-populina or Puccinia graminis induced a localized hypersensitive cell death response, indicating that CjFIT2 can recognize AvrFIT2 proteins produced by other pathogens.

Additional experiments were carried out to test other combinations of alleles of FIT2 alleles AvrFIT2 from other species. The indicated alleles of AvrFIT2 are shown in the first column, with the alleles of FIT2 are shown in the first row. Alleles were transiently co-expressed in Nicotiana tabacum leaf tissue using the methods described above.

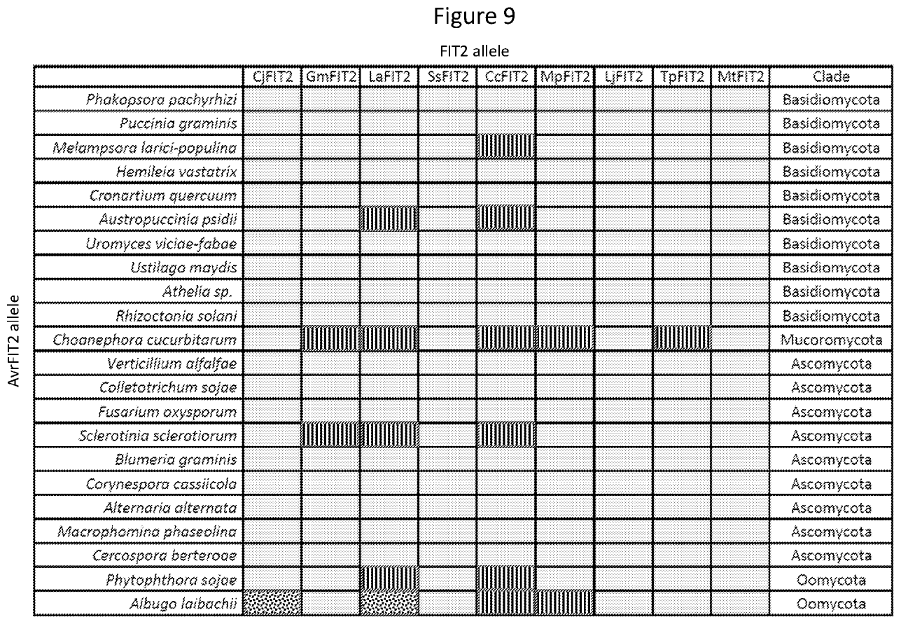

After three days, each infiltration was scored for the presence of a hypersensitive cell death response indicative of immune activation and a compatible interaction between the FIT2+AvrFIT2 alleles (FIG. 9). Grey=strong hypersensitive response, striped=moderate or weak hypersensitive response, dots=no visible response. The results indicate that all the tested AvrFIT2 alleles can be recognized by at least one FIT2 allele. Crotalaria juncea (CjFIT2), Glycine max (GmFIT2), Lupinus albus (LaFIT2), Spatholobus suberectus (SsFIT2), Cajanus cajan (CcFIT2), Mucuna pruriens (MpFIT2), Lotus japonicus (LjFIT2), Trifolium pratense (TpFIT2), and Medicago truncatula (MtFIT2).

### Example 5: Stable Expression of Crotalaria Juncea FIT2 in Glycine max

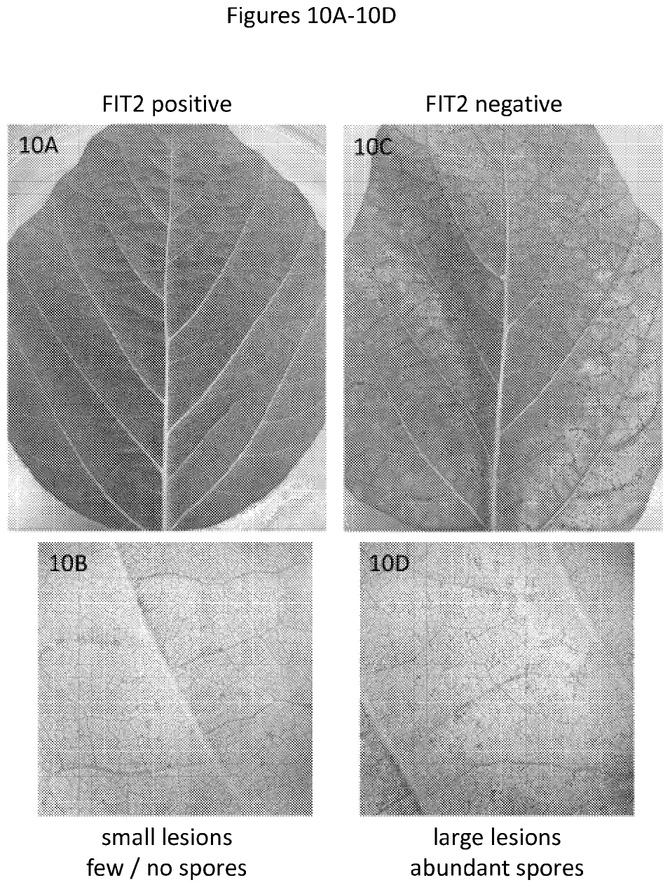

Soybean plants stably expressing CjFIT2 (SEQ ID NO: 1, example construct shown in FIG. 5) were generated and tested for resistance to ASR (Phakopsora pachyrhizi). FIGS. 10A-10D depict soybean leaves expressing CjFIT2 (FIG. 10A, close up shown in FIG. 10B) and wild type soybean leaves lacking FIT2 (FIG. 10C, close up shown in FIG. 10D) inoculated with Phakopsora pachyrhizi. The leaves were photographed at 25-days post inoculation. The wild type soybean leaves showed susceptibility to Phakopsora as seen by the development of large lesions and many fungal spores. However, soybean expressing CjFIT2 showed strong resistance to the pathogen (FIGS. 10A-10B), with small lesions and few spores.

Transgenic expression of CjFIT2 in soybean did not affect plant growth or morphology.

### Example 6: Introducing FIT2 Homologs into Other Plant Species

The FIT2 sequences isolated and described herein can be introduced into other plant species to create a plant having resistance or tolerance to a pathogen.

For example, a sequence encoding any one of the proteins of SEQ ID NOs: 2, 4, 6, 8, 10, 12, 14, 16, 18, 20, 22, 24, 26, 28, 30, 32, sequences at least 90% identical thereto, and functional homologs thereof, or sequences of SEQ ID NOs: 1, 3, 5, 7, 9, 11, 13, 15, 17, 19, 21, 23, 25, 27, 29, 31, complements thereof, fragments thereof, and sequences at least 70% identical thereto can be introduced into a plant to confer resistance or tolerance to a pathogen.

For example, as described above, Glycine max, which does possess a functional FIT2 gene but is susceptible to ASR, may be transformed with a transgene comprising SEQ ID NO: 1, or a nucleotide sequence encoding the amino acid sequence of SEQ ID NO: 2, to confer resistance to plant pathogens, such as Phakopsora pachyrhizi, that express AvrFIT2, and cause diseases like ASR. Based on the transient assays described herein, resistance to various pathogens could also be achieved with a transgene comprising any one of SEQ ID NOs: 3, 5, 7, 9, 11, 13, 15, 17, 19, 21, 23, 25, 27, 29, and/or 31, or a sequence encoding any one of the proteins of SEQ ID NOs: 2, 4, 6, 8, 10, 12, 14, 16, 18, 20, 22, 24, 26, 28, 30, and/or 32. Alternatively, the regulatory elements upstream of GmFIT2 may be targeted for genetic engineering to increase expression levels of endogenous GmFIT2 to confer resistance or tolerance to a plant pathogen.

Additional plant species susceptible to pathogens expressing AvrFIT2 proteins could also be transformed with any of the FIT2 sequences disclosed herein to confer resistance to a pathogen, including, but not limited to, corn (Zea mays), Brassica spp. (e.g., Brassica napus, Brassica rapa, Brassica juncea), alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), broad beans (Vicia faba), tobacco (Nicotiana tabacum), potato (Solanum tuberosum), tomato (Solanum lycopersicum), eggplant (Solanum melongena), spinach (Spinacia oleracea), cannabis (Cannabis spp.), melon (Cucumis melo), peanuts (Arachis hypogaea), cotton (Gossypium barbadense, Gossypium hirsutum), quince (Cydonia), sweet potato (Ipomoea batatas), cassava (Manihot esculenta), coffee (Coffea spp.), coconut (Cocos nucifera), pineapple (Ananas comosus), citrus trees (Citrus spp.), apple (Malus spp.), medlar (Mespilus), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), pear (Pyrus), cashew (Anacardium occidentale), macadamia (Macadamia integrifolia), almond (Prunus amygdalus), sugar beets (Beta vulgaris), sugarcane (Saccharum spp.), oats (for example Avena sativa), barley (for example Hordeum vulgare), vegetables and herbs (for example onion, leek, garlic peppermint), ornamentals (for example, Chrysanthemum, Fuchsia spp., Pelargonium, Rosa spp. Primula vulgaris), red cedar (Juniperus virginiana), and conifers (for example juniper (Juniperus communis)).

### Example 7: Methods of Identifying Pathogen Resistant Genes

FIT2 orthologs are likely present in additional species and genera within the Fabaceae family. FIT2 orthologs may identified by any number of means known in the art. This includes sequencing the genome or transcriptome of a plant species, identifying FIT2 homologs using a BLAST search, identifying putative FIT2 orthologs by constructing a phylogenetic tree of the homologous proteins, and then testing the identified putative FIT2 genes for AvrFIT2 recognition activity using a transient assay such as those shown and described herein. Alternatively, synthetic alleles of FIT2 may be designed by combining fragments of naturally occurring FIT2 alleles or by introducing amino acid substitutions at positions shown to be variable in an alignment of functional FIT2 proteins and similarly tested for functionality by transient assay. Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237-44, 1988); Higgins and Sharp (CABIOS, 5:151-53, 1989); Corpet et al. (Nuc. Acids Res., 16:10881-90, 1988); Huang et al. (Comp. Appls Biosci., 8:155-65, 1992); and Pearson et al. (Meth. Mol. Biol., 24:307-31, 1994). Altschul et al. (Nature Genet., 6:119-29, 1994) presents a detailed consideration of sequence alignment methods and homology calculations.

The ability of other potential FIT2 homologs or synthetic genes to function for AvrFIT2 perception can be easily and quickly tested using the transient expression assays shown and described herein or similar methods well known in the art. For example, once a FIT2 ortholog is identified in a plant species that is resistant to ASR, the gene can be cloned and tested for recognition of AvrFIT2 using transient expression assays and the PpAvrFIT2 (SEQ ID NO: 33) described herein. Examples of leaf tissue suitable for use in a FIT2-PpAvrFIT2 assay include, but are not limited to, species or accessions of Nicotiana, Solanum, Physalis, Capsicum, Lactuca, Alysicarpus, Astragalus, Baptisia, Calopogonium, Caragana, Centrosema, Cologania, Desmodium, Genista, Glycyrrhiza, Indigofera, Kummerowia, Lablab, Lespedeza, Macroptilium, Macrotyloma, Neonotonia, Pachyrhizus, Pisum, Phaseolus, Pseudovigna, Psoralea, Robinia, Senna, Sesbania, Strophostyles, Tephrosia, Teramnus, Vicia, Vigna, and Voandzeia that lack a functional native FIT2 gene. Similarly, resistance genes for other pathogens may be identified using this same method, wherein a potential gene is identified, cloned, and tested in transient assays with a protein from the target pathogen.

### BRIEF DESCRIPTION OF THE SEQUENCE LISTINGS

SEQ ID NO: 1 shows the nucleic acid sequence of Crotalariajuncea FIT2.
SEQ ID NO: 2 shows the corresponding amino acid sequence of SEQ ID NO: 1.
SEQ ID NO: 3 shows the nucleic acid sequence of Lupinus angustifolius FIT2.
SEQ ID NO: 4 shows the corresponding amino acid sequence of SEQ ID NO: 3.
SEQ ID NO: 5 shows the nucleic acid sequence of Lupinus albus FIT2.
SEQ ID NO: 6 shows the corresponding amino acid sequence of SEQ ID NO: 5.
SEQ ID NO: 7 shows the nucleic acid sequence of Spatholobus suberectus FIT2.
SEQ ID NO: 8 shows the corresponding amino acid sequence of SEQ ID NO: 7.
SEQ ID NO: 9 shows the nucleic acid sequence of Cajanus cajan FIT2.
SEQ ID NO: 10 shows the corresponding amino acid sequence of SEQ ID NO: 9.
SEQ ID NO: 11 shows the nucleic acid sequence of Mucuna pruriens FIT2.
SEQ ID NO: 12 shows the corresponding amino acid sequence of SEQ ID NO: 11.
SEQ ID NO: 13 shows the nucleic acid sequence of Glycine soja FIT2
SEQ ID NO: 14 shows the corresponding amino acid sequence of SEQ ID NO: 13.
SEQ ID NO: 15 shows the nucleic acid sequence of Glycine max FIT2.
SEQ ID NO: 16 shows the corresponding amino acid sequence of SEQ ID NO: 15.
SEQ ID NO: 17 shows the nucleic acid sequence of Trifolium pratense FIT2.
SEQ ID NO: 18 shows the corresponding amino acid sequence of SEQ ID NO: 17.
SEQ ID NO: 19 shows the nucleic acid sequence of Medicago truncatula FIT2.
SEQ ID NO: 20 shows the corresponding amino acid sequence of SEQ ID NO: 19.
SEQ ID NO: 21 shows the nucleic acid sequence of Lotus japonicus FIT2.
SEQ ID NO: 22 shows the corresponding amino acid sequence of SEQ ID NO: 21.
SEQ ID NO: 23 shows the nucleic acid sequence of Prosopis alba (XP_028800138.1) FIT2.
SEQ ID NO: 24 shows the corresponding amino acid sequence of SEQ ID NO: 23.
SEQ ID NO: 25 shows the nucleic acid sequence of Prosopis alba (XP_028802438.1) FIT2.
SEQ ID NO: 26 shows the corresponding amino acid sequence of SEQ ID NO: 25.
SEQ ID NO: 27 shows the nucleic acid sequence of Prosopis alba (XP_028763131.1) FIT2.
SEQ ID NO: 28 shows the corresponding amino acid sequence of SEQ ID NO: 27.
SEQ ID NO: 29 shows the nucleic acid sequence of Prosopis alba (XP_028774409.1) FIT2.
SEQ ID NO: 30 shows the corresponding amino acid sequence of SEQ ID NO: 29.
SEQ ID NO: 31 shows the nucleic acid sequence of Prosopis alba (XP_028756320.1) FIT2.
SEQ ID NO: 32 shows the corresponding amino acid sequence of SEQ ID NO: 31.
SEQ ID NO: 33 shows the nucleic acid sequence of Phakopsora pachyrhizi AvrFIT2.
SEQ ID NO: 34 shows the corresponding amino acid sequence of SEQ ID NO: 33.
SEQ ID NO: 35 shows the amino acid sequence of Vigna unguiculata FIT1.
SEQ ID NO: 36 shows the amino acid sequence of Nicotiana glutinosa N.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### Numbered Embodiments

1. An isolated, recombinant, or synthetic polynucleotide comprising a nucleic acid sequence encoding SEQ ID NO: 2, or a functional FIT2 protein homologous to SEQ ID NO: 2.

2. The isolated, recombinant, or synthetic polynucleotide of embodiment 1, wherein the polynucleotide encodes a functional protein having at least 50% identity to SEQ ID NO: 2.

3. The isolated, recombinant, or synthetic polynucleotide of embodiment 2, wherein the polynucleotide encodes a FIT2 protein from Prosopis alba.

4. The isolated, recombinant, or synthetic polynucleotide of embodiment 3, wherein the polynucleotide comprises SEQ ID NO: 23, a polynucleotide encoding SEQ ID NO: 24, complements thereof, or fragments thereof.

5. The isolated, recombinant, or synthetic polynucleotide of embodiment 3, wherein the polynucleotide comprises SEQ ID NO: 25, a polynucleotide encoding SEQ ID NO: 26, complements thereof, or fragments thereof.

6. The isolated, recombinant, or synthetic polynucleotide of embodiment 3, wherein the polynucleotide comprises SEQ ID NO: 27, a polynucleotide encoding SEQ ID NO: 28, complements thereof, or fragments thereof.

7. The isolated, recombinant, or synthetic polynucleotide of embodiment 3, wherein the polynucleotide comprises SEQ ID NO: 29, a polynucleotide encoding SEQ ID NO: complements thereof, or fragments thereof.

8. The isolated, recombinant, or synthetic polynucleotide of embodiment 3, wherein the polynucleotide comprises SEQ ID NO: 31, a polynucleotide encoding SEQ ID NO: 32, complements thereof, or fragments thereof.

9. The isolated, recombinant, or synthetic polynucleotide of embodiment 1, wherein the polynucleotide encodes a protein having at least 75% identity to SEQ ID NO: 2.

10. The isolated, recombinant, or synthetic polynucleotide of embodiment 9, wherein the polynucleotide encodes a FIT2 protein from Lotusjaponicus.

11. The isolated, recombinant, or synthetic polynucleotide of embodiment 10, wherein the polynucleotide comprises SEQ ID NO: 21, a polynucleotide encoding SEQ ID NO: 22, complements thereof, or fragments thereof.

12. The isolated, recombinant, or synthetic polynucleotide of embodiment 9, wherein the polynucleotide encodes a FIT2 protein from Medicago truncatula.

13. The isolated, recombinant, or synthetic polynucleotide of embodiment 12, wherein the polynucleotide comprises SEQ ID NO: 19, a polynucleotide encoding SEQ ID NO: 20, complements thereof, or fragments thereof.

14. The isolated, recombinant, or synthetic polynucleotide of embodiment 9, wherein the polynucleotide encodes a FIT2 protein from Trifolium pratense.

15. The isolated, recombinant, or synthetic polynucleotide of embodiment 14, wherein the polynucleotide comprises SEQ ID NO: 17, a polynucleotide encoding SEQ ID NO: 18, complements thereof, or fragments thereof.

16. The isolated, recombinant, or synthetic polynucleotide of embodiment 9, wherein the polynucleotide encodes a FIT2 protein from Glycine max.

17. The isolated, recombinant, or synthetic polynucleotide of embodiment 16, wherein the polynucleotide comprises SEQ ID NO: 15, a polynucleotide encoding SEQ ID NO: 16, complements thereof, or fragments thereof.

18. The isolated, recombinant, or synthetic polynucleotide of embodiment 9, wherein the polynucleotide encodes a FIT2 protein from Glycine soja.

19. The isolated, recombinant, or synthetic polynucleotide of embodiment 18, wherein the polynucleotide comprises SEQ ID NO: 13, a polynucleotide encoding SEQ ID NO: 14, complements thereof, or fragments thereof.

20. The isolated, recombinant, or synthetic polynucleotide of embodiment 9, wherein the polynucleotide encodes a FIT2 protein from Mucunapuriens.

21. The isolated, recombinant, or synthetic polynucleotide of embodiment 20, wherein the polynucleotide comprises SEQ ID NO: 11, a polynucleotide encoding SEQ ID NO: 12, complements thereof, or fragments thereof.

22. The isolated, recombinant, or synthetic polynucleotide of embodiment 9, wherein the polynucleotide encodes a FIT2 protein from Cajanus cajan.

23. The isolated, recombinant, or synthetic polynucleotide of embodiment 22, wherein the polynucleotide comprises SEQ ID NO: 9, a polynucleotide encoding SEQ ID NO: 10, complements thereof, or fragments thereof.

24. The isolated, recombinant, or synthetic polynucleotide of embodiment 1, wherein the polynucleotide encodes a protein having at least 80% identity to SEQ ID NO: 2.

25. The isolated, recombinant, or synthetic polynucleotide of embodiment 24, wherein the polynucleotide encodes a FIT2 protein from Spatholobus suberectus.

26. The isolated, recombinant, or synthetic polynucleotide of embodiment 25, wherein the polynucleotide comprises SEQ ID NO: 7, a polynucleotide encoding SEQ ID NO: 8, complements thereof, or fragments thereof.

27. The isolated, recombinant, or synthetic polynucleotide of embodiment 1, wherein the polynucleotide encodes a protein having at least 85% identity to SEQ ID NO: 2.

28. The isolated, recombinant, or synthetic polynucleotide of embodiment 27, wherein the polynucleotide encodes a FIT2 protein from Lupinus albus.

29. The isolated, recombinant, or synthetic polynucleotide of embodiment 28, wherein the polynucleotide comprises SEQ ID NO: 5, a polynucleotide encoding SEQ ID NO: 6, complements thereof, or fragments thereof.

30. The isolated, recombinant, or synthetic polynucleotide of embodiment 27, wherein the polynucleotide encodes a FIT2 protein from Lupinus angustifoliuss.

31. The isolated, recombinant, or synthetic polynucleotide of embodiment 30, wherein the polynucleotide comprises SEQ ID NO: 3, a polynucleotide encoding SEQ ID NO: 4, complements thereof, or fragments thereof.

32. The isolated, recombinant, or synthetic polynucleotide of embodiment 1, wherein the polynucleotide encodes a protein having at least 90% identity to SEQ ID NO: 2.

33. The isolated, recombinant, or synthetic polynucleotide of embodiment 1, wherein the polynucleotide encodes a protein having at least 95% identity to SEQ ID NO: 2.

34. The isolated, recombinant, or synthetic polynucleotide of embodiment 1, wherein the polynucleotide encodes a protein having at least 96% identity to SEQ ID NO: 2.

35. The isolated, recombinant, or synthetic polynucleotide of embodiment 1, wherein the polynucleotide encodes a protein having at least 97% identity to SEQ ID NO: 2.

36. The isolated, recombinant, or synthetic polynucleotide of embodiment 1, wherein the polynucleotide encodes a protein having at least 98% identity to SEQ ID NO: 2.

37. The isolated, recombinant, or synthetic polynucleotide of embodiment 1, wherein the polynucleotide encodes a protein having at least 99% identity to SEQ ID NO: 2.

38. An isolated, recombinant, or synthetic polynucleotide comprising a nucleic acid sequence encoding a FIT2 protein, wherein the protein is selected from the group consisting of: SEQ ID NOs: 2, 4, 6, 8, 10, 12, 14, 16, 18, 20, 22, 24, 26, 28, 30, 32, and functional homologs thereof.

39. The isolated, recombinant, or synthetic polynucleotide of embodiment 38, wherein the polynucleotide comprises a nucleic acid sequence selected from the group consisting of SEQ ID NOs: 1, 3, 5, 7, 9, 11, 13, 15, 17, 19, 21, 23, 25, 27, 29, 31, complements thereof, fragments thereof, and sequences at least 70% identical thereto.

40. A genetic construct comprising at least one of the nucleic acid sequences of any one of embodiments 1-39.

41. A plant, plant part, or plant cell transformed with at least one of the nucleic acid sequences of any one of embodiments 1-39 or the genetic construct of embodiment 40, wherein said plant, plant part or plant cell is resistant or tolerant to a pathogen.

42. The plant, plant part, or plant cell of embodiment 41, wherein the pathogen is a fungus from the order Cantharellales, Mucorales, Ustilaginales, Atheliales, or Pucciniales.

43. The plant, plant part, or plant cell of embodiment 41 or 42, wherein the fungal pathogen is Choanephora cucurbitarum, Ustilago maydis, Athelia spp., Rhizoctonia solani, Melampsora spp., Phakopsora pachyrhizi, Phakopsora meibomiae, Phakopsora euvitis, Phakopsora spp., Puccinia spp., Uromyces spp., Austropuccinia spp., Cronartium spp. Hemileia vastatri.

44. The plant, plant part, or plant cell of any one of embodiments 41-43, wherein the plant, plant part, or plant cell is in the subfamily Papilionoideae.

45. The plant, plant part, or plant cell of any one of embodiments 41-44, wherein the plant, plant part, or plant cell is Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

46. The plant, plant part, or plant cell of any one of embodiments 41-45, wherein the plant, plant part, or plant cell is Glycine max, and wherein the plant, plant part, or plant cell is resistant to Asian Soybean Rust caused by Phakopsorapachyrhizi.

47. The plant, plant part, or plant cell of embodiment 46, wherein the resistance to Asian Soybean Rust is conferred by a transgene comprising SEQ ID NO: 1, or a nucleotide sequence encoding the amino acid sequence of SEQ ID NO: 2.

48. The plant, plant part, or plant cell of embodiment 46, wherein the resistance to Asian Soybean Rust is conferred by a transgene comprising SEQ ID NO: 3, or a nucleotide sequence encoding the amino acid sequence of SEQ ID NO: 4.

49. The plant, plant part, or plant cell of embodiment 46, wherein the resistance to Asian Soybean Rust is conferred by a transgene comprising SEQ ID NO: 5, or a nucleotide sequence encoding the amino acid sequence of SEQ ID NO: 6.

50. The plant, plant part, or plant cell of embodiment 46, wherein the resistance to Asian Soybean Rust is conferred by a transgene comprising SEQ ID NO: 7, or a nucleotide sequence encoding the amino acid sequence of SEQ ID NO: 8.

51. The plant, plant part, or plant cell of embodiment 46, wherein the resistance to Asian Soybean Rust is conferred by a transgene comprising SEQ ID NO: 9, or a nucleotide sequence encoding the amino acid sequence of SEQ ID NO: 10.

52. The plant, plant part, or plant cell of embodiment 46, wherein the resistance to Asian Soybean Rust is conferred by a transgene comprising SEQ ID NO: 11, or a nucleotide sequence encoding the amino acid sequence of SEQ ID NO: 12.

53. The plant, plant part, or plant cell of embodiment 46, wherein the resistance to Asian Soybean Rust is conferred by a transgene comprising SEQ ID NO: 13, or a nucleotide sequence encoding the amino acid sequence of SEQ ID NO: 14.

54. The plant, plant part, or plant cell of embodiment 46, wherein the resistance to Asian Soybean Rust is conferred by a transgene comprising SEQ ID NO: 15, or a nucleotide sequence encoding the amino acid sequence of SEQ ID NO: 16.

55. The plant, plant part, or plant cell of embodiment 46, wherein the resistance to Asian Soybean Rust is conferred by a transgene comprising SEQ ID NO: 17, or a nucleotide sequence encoding the amino acid sequence of SEQ ID NO: 18.

56. The plant, plant part, or plant cell of embodiment 46, wherein the resistance to Asian Soybean Rust is conferred by a transgene comprising SEQ ID NO: 19, or a nucleotide sequence encoding the amino acid sequence of SEQ ID NO: 20.

57. The plant, plant part, or plant cell of embodiment 46, wherein the resistance to Asian Soybean Rust is conferred by a transgene comprising SEQ ID NO: 21, or a nucleotide sequence encoding the amino acid sequence of SEQ ID NO: 22.

58. The plant, plant part, or plant cell of embodiment 46, wherein the resistance to Asian Soybean Rust is conferred by a transgene comprising SEQ ID NO: 23, or a nucleotide sequence encoding the amino acid sequence of SEQ ID NO: 24.

59. The plant, plant part, or plant cell of embodiment 46, wherein the resistance to Asian Soybean Rust is conferred by a transgene comprising SEQ ID NO: 25, or a nucleotide sequence encoding the amino acid sequence of SEQ ID NO: 26.

60. The plant, plant part, or plant cell of embodiment 46, wherein the resistance to Asian Soybean Rust is conferred by a transgene comprising SEQ ID NO: 27, or a nucleotide sequence encoding the amino acid sequence of SEQ ID NO: 28.

61. The plant, plant part, or plant cell of embodiment 46, wherein the resistance to Asian Soybean Rust is conferred by a transgene comprising SEQ ID NO: 29, or a nucleotide sequence encoding the amino acid sequence of SEQ ID NO: 30.

62. The plant, plant part, or plant cell of embodiment 46, wherein the resistance to Asian Soybean Rust is conferred by a transgene comprising SEQ ID NO: 31, or a nucleotide sequence encoding the amino acid sequence of SEQ ID NO: 32.

63. A method of producing a plant, plant part, or plant cell having resistance or tolerance to a pathogen, wherein the method comprises:
transforming a plant, plant part, or plant cell with a nucleotide sequence encoding a Toll-like Interleukin-1 Receptor (TIR) Nucleotide binding, Leucine-rich Repeat (NLR) immune receptor protein, wherein said immune receptor protein mediates the perception of the pathogen protein AvrFIT2 or homologs thereof, and
wherein expression of the immune receptor protein prevents the pathogen from colonizing the plant, or prevents the pathogen from affecting plant growth or yield.

64. The method of embodiment 63, wherein the pathogen protein comprises SEQ ID NO: 34, or sequences at least 90% identical thereto.

65. The method of embodiment 63 or 64, wherein the nucleotide sequence encoding the immune receptor protein has been codon optimized.

66. The method of any one of embodiments 63-65, wherein the immune receptor protein is selected from the group consisting of:
an isolated, recombinant, or synthetic polynucleotide comprising a nucleic acid sequence encoding a FIT2 protein, wherein the protein is selected from the group consisting of: SEQ ID NOs: 2, 4, 6, 8, 10, 12, 14, 16, 18, 20, 22, 24, 26, 28, 30, 32, and functional homologs thereof, or
an isolated, recombinant, or synthetic polynucleotide encoding a FIT2 protein, wherein the nucleic acid sequence is selected from the group consisting of SEQ ID NOs: 1, 3, 5, 7, 9, 11, 13, 15, 17, 19, 21, 23, 25, 27, 29, 31, complements thereof, fragments thereof, and sequences at least 70% identical thereto.

67. The method of any one of embodiments 63-66, wherein the plant, plant part, or plant cell is transformed with two or more nucleotide sequences encoding immune receptor proteins.

68. The method of embodiment 67, wherein the two or more nucleotide sequences encoding immune receptor proteins comprise FIT1 and FIT2.

69. The method of any one of embodiments 63-68, wherein the plant, plant part, or plant cell is transformed with one or more additional desired traits

70. The method of embodiment 69, wherein the one or more additional desired traits are stacked together with the immune receptor protein on the same DNA construct.

71. The method of any one of embodiments 63-70, further comprising introgressing one or more additional desired traits.

72. The method of any one of embodiments 63-71, wherein the one or more additional desired traits are resistance traits to a disease, pest, or abiotic stress.

73. A plant, plant part, or plant cell produced by the method of any one of embodiments 63-72, wherein the plant, plant part, or plant cell is resistant to a pathogen.

74. A plant, plant part, or plant cell produced by the method of any one of embodiments 63-72, wherein the plant, plant part, or plant cell is tolerant to a pathogen.

75. The plant, plant part, or plant cell of embodiment 73 or 74, wherein the immune receptor protein is transiently expressed.

76. The plant, plant part, or plant cell of embodiment 73 or 74, wherein the immune receptor protein is stably expressed.

77. The plant, plant part, or plant cell of any one of embodiments 63-76, wherein the plant, plant part, or plant cell is in the subfamily Papilionoideae.

78. The plant, plant part, or plant cell of embodiment 77, wherein the plant, plant part, or plant cell is Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

79. The plant, plant part, or plant cell of any one of embodiments 73-78, wherein the plant is Glycine max, wherein the plant, plant part, or plant cell is resistant to Asian Soybean Rust caused by Phakopsorapachyrhizi.

80. A method of genetically engineering a pathogen resistance or tolerance trait in a plant, plant part, or plant cell, comprising:
providing a plant species that is susceptible to a pathogen;
identifying within the genome of the plant species a homolog of FIT2; and
genetically modifying a plant, plant part, or plant cell from the susceptible plant species with targeted gene editing, wherein said targeted gene editing confers resistance or tolerance to a pathogen.

81. The method of embodiment 80, wherein said targeted gene editing is directed at the FIT2 homolog, promotor, and/or a regulatory sequence controlling expression of the FIT2 homolog.

82. The method of embodiment 80 or 81, wherein the targeted gene editing uses an engineered or natural nuclease selected from the group consisting of homing endonucleases/meganucleases (EMNs), zinc finger nucleases (ZFNs), and transcription activator-like effector nucleases (TALENs).

83. The method of embodiment 80 or 81, wherein targeted gene editing uses a clustered regularly interspaced short palindromic repeats (CRISPR)-Cas nuclease.

84. The method of embodiment 83, wherein the nuclease is selected from the group consisting of Cas9, Cas12, Cas13, CasX, and CasY.

85. The method of embodiment 80, wherein the targeted gene editing is inserting a promoter to alter expression of the FIT2 homolog.

86. The method of embodiment 85, wherein the promoter increases expression of the FIT2 homolog.

87. The method of embodiment 85, wherein the promoter alters the location of expression of the FIT2 homolog.

88. The method of any one of embodiments 80-87, further comprising breeding with, or asexually propagating the plant.

89. A genetically modified plant, plant part, or plant cell produced by the method of any one of embodiments 80-88, wherein said plant, plant part, or plant cell exhibits resistance or tolerance to a pathogen.

90. The genetically modified plant, plant part, or plant cell of embodiment 89, wherein the pathogen is a fungus from the order Cantharellales, Mucorales, Ustilaginales, Atheliales, or Pucciniales.

91. The genetically modified plant, plant part, or plant cell of embodiment 90, wherein the fungal pathogen is Choanephora cucurbitarum, Ustilago maydis, Athelia spp., Rhizoctonia solani, Melampsora spp., Phakopsora pachyrhizi, Phakopsora meibomiae, Phakopsora euvitis, Phakopsora spp., Puccinia spp., Uromyces spp., Austropuccinia spp., Cronartium spp. Hemileia vastatri.

92. The genetically modified plant, plant part, or plant cell of any one of embodiments 89-91, wherein the fungal pathogen is Phakopsora pachyrhizi and the plant, plant part, or plant cell is Glycine max.

93. A method for identifying a functional FIT2 gene and/or allele thereof comprising:
isolating a FIT2 homolog or allele thereof,
expressing all or a substantial fragment of said FIT2 homolog or allele thereof in combination with a homolog of AvrFIT2 in a plant, plant part, or plant cell; and
assaying said plant, plant part, or plant cell for an immune response.

94. The method of embodiment 93, wherein the protein comprises SEQ ID NO: 34, or sequences at least 90% identical thereto.

95. The method of embodiment 93 or 94, wherein the FIT2 allele is a synthetic variant.

96. The method of any one of embodiments 93-95, wherein the plant, plant part, or plant cell is a species of Nicotiana, Solanum, Physalis, Capsicum, Lactuca, Alysicarpus, Astragalus, Baptisia, Calopogonium, Caragana, Centrosema, Cologania, Crotalaria, Desmodium, Genista, Glycine, Glycyrrhiza, Indigofera, Kummerowia, Lablab, Lathyrus, Lespedeza, Macroptilium, Macrotyloma, Neonotonia, Pachyrhizus, Pisum, Phaseolus, Pseudovigna, Psoralea, Robinia, Senna, Sesbania, Strophostyles, Tephrosia, Teramnus, Vicia, Vigna, and Voandzeia.

97. A transgenic plant, plant part, or plant cell having resistance or tolerance to a plant pathogen, wherein the resistance is conferred by a transgene comprising SEQ ID NO: 1, or a sequence at least 70% identical thereto.

98. A transgenic plant, plant part, or plant cell having resistance or tolerance to a plant pathogen, wherein the resistance is conferred by a transgene comprising SEQ ID NO: 1, or a sequence at least 80% identical thereto.

99. A transgenic plant, plant part, or plant cell having resistance or tolerance to a plant pathogen, wherein the resistance is conferred by a transgene comprising SEQ ID NO: 1, or a sequence at least 90% identical thereto.

100. A transgenic plant, plant part, or plant cell having resistance or tolerance to a plant pathogen, wherein the resistance is conferred by a transgene comprising a nucleotide sequence encoding the amino acid sequence of SEQ ID NO: 2, or an amino acid sequence at least 90% identical thereto.

101. A transgenic plant, plant part, or plant cell having resistance or tolerance to a plant pathogen, wherein the resistance is conferred by a transgene comprising SEQ ID NO: 3, or a sequence at least 70% identical thereto.

102. A transgenic plant, plant part, or plant cell having resistance or tolerance to a plant pathogen, wherein the resistance is conferred by a transgene comprising SEQ ID NO: 3, or a sequence at least 80% identical thereto.

103. A transgenic plant, plant part, or plant cell having resistance or tolerance to a plant pathogen, wherein the resistance is conferred by a transgene comprising SEQ ID NO: 3, or a sequence at least 90% identical thereto.

104. A transgenic plant, plant part, or plant cell having resistance or tolerance to a plant pathogen, wherein the resistance is conferred by a transgene comprising a nucleotide sequence encoding the amino acid sequence of SEQ ID NO: 4, or an amino acid sequence at least 90% identical thereto.

105. A transgenic plant, plant part, or plant cell having resistance or tolerance to a plant pathogen, wherein the resistance is conferred by a transgene comprising SEQ ID NO: 5, or a sequence at least 70% identical thereto.

106. A transgenic plant, plant part, or plant cell having resistance or tolerance to a plant pathogen, wherein the resistance is conferred by a transgene comprising SEQ ID NO: 5, or a sequence at least 80% identical thereto.

107. A transgenic plant, plant part, or plant cell having resistance or tolerance to a plant pathogen, wherein the resistance is conferred by a transgene comprising SEQ ID NO: 5, or a sequence at least 90% identical thereto.

108. A transgenic plant, plant part, or plant cell having resistance or tolerance to a plant pathogen, wherein the resistance is conferred by a transgene comprising a nucleotide sequence encoding the amino acid sequence of SEQ ID NO: 6, or an amino acid sequence at least 90% identical thereto.

109. A transgenic plant, plant part, or plant cell having resistance or tolerance to a plant pathogen, wherein the resistance is conferred by a transgene comprising SEQ ID NO: 7, or a sequence at least 70% identical thereto.

110. A transgenic plant, plant part, or plant cell having resistance or tolerance to a plant pathogen, wherein the resistance is conferred by a transgene comprising SEQ ID NO: 7, or a sequence at least 80% identical thereto.

111. A transgenic plant, plant part, or plant cell having resistance or tolerance to a plant pathogen, wherein the resistance is conferred by a transgene comprising SEQ ID NO: 7, or a sequence at least 90% identical thereto.

112. A transgenic plant, plant part, or plant cell having resistance or tolerance to a plant pathogen, wherein the resistance is conferred by a transgene comprising a nucleotide sequence encoding the amino acid sequence of SEQ ID NO: 8, or an amino acid sequence at least 90% identical thereto.

113. A transgenic plant, plant part, or plant cell having resistance or tolerance to a plant pathogen, wherein the resistance is conferred by a transgene comprising SEQ ID NO: 9, or a sequence at least 70% identical thereto.

114. A transgenic plant, plant part, or plant cell having resistance or tolerance to a plant pathogen, wherein the resistance is conferred by a transgene comprising SEQ ID NO: 9, or a sequence at least 80% identical thereto.

115. A transgenic plant, plant part, or plant cell having resistance or tolerance to a plant pathogen, wherein the resistance is conferred by a transgene comprising SEQ ID NO: 9, or a sequence at least 90% identical thereto.

116. A transgenic plant, plant part, or plant cell having resistance or tolerance to a plant pathogen, wherein the resistance is conferred by a transgene comprising a nucleotide sequence encoding the amino acid sequence of SEQ ID NO: 10, or an amino acid sequence at least 90% identical thereto.

117. A transgenic plant, plant part, or plant cell having resistance or tolerance to a plant pathogen, wherein the resistance is conferred by a transgene comprising SEQ ID NO: 11, or a sequence at least 70% identical thereto.

118. A transgenic plant, plant part, or plant cell having resistance or tolerance to a plant pathogen, wherein the resistance is conferred by a transgene comprising SEQ ID NO: 11, or a sequence at least 80% identical thereto.

119. A transgenic plant, plant part, or plant cell having resistance or tolerance to a plant pathogen, wherein the resistance is conferred by a transgene comprising SEQ ID NO: 11, or a sequence at least 90% identical thereto.

120. A transgenic plant, plant part, or plant cell having resistance or tolerance to a plant pathogen, wherein the resistance is conferred by a transgene comprising a nucleotide sequence encoding the amino acid sequence of SEQ ID NO: 12, or an amino acid sequence at least 90% identical thereto.

121. A transgenic plant, plant part, or plant cell having resistance or tolerance to a plant pathogen, wherein the resistance is conferred by a transgene comprising SEQ ID NO: 13, or a sequence at least 70% identical thereto.

122. A transgenic plant, plant part, or plant cell having resistance or tolerance to a plant pathogen, wherein the resistance is conferred by a transgene comprising SEQ ID NO: 13, or a sequence at least 80% identical thereto.

123. A transgenic plant, plant part, or plant cell having resistance or tolerance to a plant pathogen, wherein the resistance is conferred by a transgene comprising SEQ ID NO: 13, or a sequence at least 90% identical thereto.

124. A transgenic plant, plant part, or plant cell having resistance or tolerance to a plant pathogen, wherein the resistance is conferred by a transgene comprising a nucleotide sequence encoding the amino acid sequence of SEQ ID NO: 14, or an amino acid sequence at least 90% identical thereto.

125. A transgenic plant, plant part, or plant cell having resistance or tolerance to a plant pathogen, wherein the resistance is conferred by a transgene comprising SEQ ID NO: 15, or a sequence at least 70% identical thereto.

126. A transgenic plant, plant part, or plant cell having resistance or tolerance to a plant pathogen, wherein the resistance is conferred by a transgene comprising SEQ ID NO: 15, or a sequence at least 80% identical thereto.

127. A transgenic plant, plant part, or plant cell having resistance or tolerance to a plant pathogen, wherein the resistance is conferred by a transgene comprising SEQ ID NO: 15, or a sequence at least 90% identical thereto.

128. A transgenic plant, plant part, or plant cell having resistance or tolerance to a plant pathogen, wherein the resistance is conferred by a transgene comprising a nucleotide sequence encoding the amino acid sequence of SEQ ID NO: 16, or an amino acid sequence at least 90% identical thereto.

129. A transgenic plant, plant part, or plant cell having resistance or tolerance to a plant pathogen, wherein the resistance is conferred by a transgene comprising SEQ ID NO: 17, or a sequence at least 70% identical thereto.

130. A transgenic plant, plant part, or plant cell having resistance or tolerance to a plant pathogen, wherein the resistance is conferred by a transgene comprising SEQ ID NO: 17, or a sequence at least 80% identical thereto.

131. A transgenic plant, plant part, or plant cell having resistance or tolerance to a plant pathogen, wherein the resistance is conferred by a transgene comprising SEQ ID NO: 17, or a sequence at least 90% identical thereto.

132. A transgenic plant, plant part, or plant cell having resistance or tolerance to a plant pathogen, wherein the resistance is conferred by a transgene comprising a nucleotide sequence encoding the amino acid sequence of SEQ ID NO: 18, or an amino acid sequence at least 90% identical thereto.

133. A transgenic plant, plant part, or plant cell having resistance or tolerance to a plant pathogen, wherein the resistance is conferred by a transgene comprising SEQ ID NO: 19, or a sequence at least 70% identical thereto.

134. A transgenic plant, plant part, or plant cell having resistance or tolerance to a plant pathogen, wherein the resistance is conferred by a transgene comprising SEQ ID NO: 19, or a sequence at least 80% identical thereto.

135. A transgenic plant, plant part, or plant cell having resistance or tolerance to a plant pathogen, wherein the resistance is conferred by a transgene comprising SEQ ID NO: 19, or a sequence at least 90% identical thereto.

136. A transgenic plant, plant part, or plant cell having resistance or tolerance to a plant pathogen, wherein the resistance is conferred by a transgene comprising a nucleotide sequence encoding the amino acid sequence of SEQ ID NO: 20, or an amino acid sequence at least 90% identical thereto.

137. A transgenic plant, plant part, or plant cell having resistance or tolerance to a plant pathogen, wherein the resistance is conferred by a transgene comprising SEQ ID NO: 21, or a sequence at least 70% identical thereto.

138. A transgenic plant, plant part, or plant cell having resistance or tolerance to a plant pathogen, wherein the resistance is conferred by a transgene comprising SEQ ID NO: 21, or a sequence at least 80% identical thereto.

139. A transgenic plant, plant part, or plant cell having resistance or tolerance to a plant pathogen, wherein the resistance is conferred by a transgene comprising SEQ ID NO: 21, or a sequence at least 90% identical thereto.

140. A transgenic plant, plant part, or plant cell having resistance or tolerance to a plant pathogen, wherein the resistance is conferred by a transgene comprising a nucleotide sequence encoding the amino acid sequence of SEQ ID NO: 22, or an amino acid sequence at least 90% identical thereto.

141. A transgenic plant, plant part, or plant cell having resistance or tolerance to a plant pathogen, wherein the resistance is conferred by a transgene comprising SEQ ID NO: 23, or a sequence at least 70% identical thereto.

142. A transgenic plant, plant part, or plant cell having resistance or tolerance to a plant pathogen, wherein the resistance is conferred by a transgene comprising SEQ ID NO: 23, or a sequence at least 80% identical thereto.

143. A transgenic plant, plant part, or plant cell having resistance or tolerance to a plant pathogen, wherein the resistance is conferred by a transgene comprising SEQ ID NO: 23, or a sequence at least 90% identical thereto.

144. A transgenic plant, plant part, or plant cell having resistance or tolerance to a plant pathogen, wherein the resistance is conferred by a transgene comprising a nucleotide sequence encoding the amino acid sequence of SEQ ID NO: 24, or an amino acid sequence at least 90% identical thereto.

145. A transgenic plant, plant part, or plant cell having resistance or tolerance to a plant pathogen, wherein the resistance is conferred by a transgene comprising SEQ ID NO: 25, or a sequence at least 70% identical thereto.

146. A transgenic plant, plant part, or plant cell having resistance or tolerance to a plant pathogen, wherein the resistance is conferred by a transgene comprising SEQ ID NO: 25, or a sequence at least 80% identical thereto.

147. A transgenic plant, plant part, or plant cell having resistance or tolerance to a plant pathogen, wherein the resistance is conferred by a transgene comprising SEQ ID NO: 25, or a sequence at least 90% identical thereto.

148. A transgenic plant, plant part, or plant cell having resistance or tolerance to a plant pathogen, wherein the resistance is conferred by a transgene comprising a nucleotide sequence encoding the amino acid sequence of SEQ ID NO: 26, or an amino acid sequence at least 90% identical thereto.

149. A transgenic plant, plant part, or plant cell having resistance or tolerance to a plant pathogen, wherein the resistance is conferred by a transgene comprising SEQ ID NO: 27, or a sequence at least 70% identical thereto.

150. A transgenic plant, plant part, or plant cell having resistance or tolerance to a plant pathogen, wherein the resistance is conferred by a transgene comprising SEQ ID NO: 27, or a sequence at least 80% identical thereto.

151. A transgenic plant, plant part, or plant cell having resistance or tolerance to a plant pathogen, wherein the resistance is conferred by a transgene comprising SEQ ID NO: 27, or a sequence at least 90% identical thereto.

152. A transgenic plant, plant part, or plant cell having resistance or tolerance to a plant pathogen, wherein the resistance is conferred by a transgene comprising a nucleotide sequence encoding the amino acid sequence of SEQ ID NO: 28, or an amino acid sequence at least 90% identical thereto.

153. A transgenic plant, plant part, or plant cell having resistance or tolerance to a plant pathogen, wherein the resistance is conferred by a transgene comprising SEQ ID NO: 29, or a sequence at least 70% identical thereto.

154. A transgenic plant, plant part, or plant cell having resistance or tolerance to a plant pathogen, wherein the resistance is conferred by a transgene comprising SEQ ID NO: 29, or a sequence at least 80% identical thereto.

155. A transgenic plant, plant part, or plant cell having resistance or tolerance to a plant pathogen, wherein the resistance is conferred by a transgene comprising SEQ ID NO: 29, or a sequence at least 90% identical thereto.

156. A transgenic plant, plant part, or plant cell having resistance or tolerance to a plant pathogen, wherein the resistance is conferred by a transgene comprising a nucleotide sequence encoding the amino acid sequence of SEQ ID NO: 30, or an amino acid sequence at least 90% identical thereto.

157. A transgenic plant, plant part, or plant cell having resistance or tolerance to a plant pathogen, wherein the resistance is conferred by a transgene comprising SEQ ID NO: 31, or a sequence at least 70% identical thereto.

158. A transgenic plant, plant part, or plant cell having resistance or tolerance to a plant pathogen, wherein the resistance is conferred by a transgene comprising SEQ ID NO: 31, or a sequence at least 80% identical thereto.

159. A transgenic plant, plant part, or plant cell having resistance or tolerance to a plant pathogen, wherein the resistance is conferred by a transgene comprising SEQ ID NO: 31, or a sequence at least 90% identical thereto.

160. A transgenic plant, plant part, or plant cell having resistance or tolerance to a plant pathogen, wherein the resistance is conferred by a transgene comprising a nucleotide sequence encoding the amino acid sequence of SEQ ID NO: 32, or an amino acid sequence at least 90% identical thereto.

161. A recombinant DNA construct comprising at least one of:
a nucleic acid sequence encoding a protein comprising SEQ ID NO: 2, or a protein at least 90% identical thereto; a nucleic acid sequence encoding a protein comprising SEQ ID NO: 4, or a protein at least 90% identical thereto; a nucleic acid sequence encoding a protein comprising SEQ ID NO: 6, or a protein at least 90% identical thereto; a nucleic acid sequence encoding a protein comprising SEQ ID NO: 8, or a protein at least 90% identical thereto; a nucleic acid sequence encoding a protein comprising SEQ ID NO: 10, or a protein at least 90% identical thereto; a nucleic acid sequence encoding a protein comprising SEQ ID NO: 12, or a protein at least 90% identical thereto; a nucleic acid sequence encoding a protein comprising SEQ ID NO: 14, or a protein at least 90% identical thereto; a nucleic acid sequence encoding a protein comprising SEQ ID NO: 16, or a protein at least 90% identical thereto; a nucleic acid sequence encoding a protein comprising SEQ ID NO: 18, or a protein at least 90% identical thereto; a nucleic acid sequence encoding a protein comprising SEQ ID NO: 20, or a protein at least 90% identical thereto; a nucleic acid sequence encoding a protein comprising SEQ ID NO: 22, or a protein at least 90% identical thereto; a nucleic acid sequence encoding a protein comprising SEQ ID NO: 24, or a protein at least 90% identical thereto; a nucleic acid sequence encoding a protein comprising SEQ ID NO: 26, or a protein at least 90% identical thereto; a nucleic acid sequence encoding a protein comprising SEQ ID NO: 28, or a protein at least 90% identical thereto; a nucleic acid sequence encoding a protein comprising SEQ ID NO: 30, or a protein at least 90% identical thereto; a nucleic acid sequence encoding a protein comprising SEQ ID NO: 32, or a protein at least 90% identical thereto; or combinations thereof, and wherein the construct is capable of conferring resistance to a fungal pathogen when transformed into a plant.

162. A transgenic plant, plant part, or plant cell having resistance or tolerance to a fungal pathogen, wherein the resistance or tolerance is conferred by a transgene comprising at least one of: a nucleic acid sequence encoding a protein comprising SEQ ID NO: 2, or a protein at least 90% identical thereto; a nucleic acid sequence encoding a protein comprising SEQ ID NO: 4, or a protein at least 90% identical thereto; a nucleic acid sequence encoding a protein comprising SEQ ID NO: 6, or a protein at least 90% identical thereto; a nucleic acid sequence encoding a protein comprising SEQ ID NO: 8, or a protein at least 90% identical thereto; a nucleic acid sequence encoding a protein comprising SEQ ID NO: 10, or a protein at least 90% identical thereto; a nucleic acid sequence encoding a protein comprising SEQ ID NO: 12, or a protein at least 90% identical thereto; a nucleic acid sequence encoding a protein comprising SEQ ID NO: 14, or a protein at least 90% identical thereto; a nucleic acid sequence encoding a protein comprising SEQ ID NO: 16, or a protein at least 90% identical thereto; a nucleic acid sequence encoding a protein comprising SEQ ID NO: 18, or a protein at least 90% identical thereto; a nucleic acid sequence encoding a protein comprising SEQ ID NO: 20, or a protein at least 90% identical thereto; a nucleic acid sequence encoding a protein comprising SEQ ID NO: 22, or a protein at least 90% identical thereto; a nucleic acid sequence encoding a protein comprising SEQ ID NO: 24, or a protein at least 90% identical thereto; a nucleic acid sequence encoding a protein comprising SEQ ID NO: 26, or a protein at least 90% identical thereto; a nucleic acid sequence encoding a protein comprising SEQ ID NO: 28, or a protein at least 90% identical thereto; a nucleic acid sequence encoding a protein comprising SEQ ID NO: 30, or a protein at least 90% identical thereto; a nucleic acid sequence encoding a protein comprising SEQ ID NO: 32, or a protein at least 90% identical thereto; or combinations thereof.

163. The transgenic plant, plant part, or plant cell of embodiment 162, wherein the fungal pathogen is from the order Pucciniales or Cantharellales.

164. The transgenic plant, plant part, or plant cell of embodiment 163, wherein the fungal pathogen is Phakopsora pachyrhizi, Rhizoctonia solani, Melampsora spp., Phakopsora meibomiae, Phakopsora euvitis, Phakopsora spp., Puccinia spp., Uromyces spp., Austropuccinia spp., Cronartium spp., Austropuccinia spp., Cronartium spp. or Hemileia vastatrix.

165. The transgenic plant, plant part, or plant cell of embodiment 162, wherein the plant, plant part, or plant cell is in the subfamily Papilionoideae.

166. The transgenic plant, plant part, or plant cell of embodiment 162, wherein the plant, plant part, or plant cell is Glycine spp., Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

167. The transgenic plant, plant part, or plant cell of embodiment 166, wherein the plant, plant part, or plant cell is Glycine max, and wherein the plant, plant part, or plant cell is resistant to Asian Soybean Rust caused by Phakopsorapachyrhizi.

168. The transgenic plant, plant part, or plant cell of embodiment 167, wherein the resistance to Asian Soybean Rust is conferred by a transgene comprising SEQ ID NO: 1, or by a sequence at least 70% identical thereto.

169. A method of producing a plant, plant part, or plant cell having resistance or tolerance to a fungal pathogen, wherein the method comprises: transforming a plant, plant part, or plant cell with an isolated, recombinant, or synthetic polynucleotide comprising: a nucleic acid sequence encoding a functional FIT2 protein, wherein the nucleic acid sequence is at least 70% identical to SEQ ID NO: 1 and selecting a plant comprising the polynucleotide and having resistance or tolerance to a fungal pathogen.

170. The method of embodiment 169, wherein the nucleotide sequence encoding the FIT2 protein has been codon optimized.

171. The method of embodiment 169, wherein the plant, plant part, or plant cell is transformed with two or more polynucleotides encoding different FIT2 proteins.

172. The method of embodiment 169, wherein the plant, plant part, or plant cell is transformed or introgressed with one or more additional desired traits.

173. The method of embodiment 169, wherein the one or more additional desired traits are resistance traits to a disease, pest, or abiotic stress.

174. A plant, plant part, or plant cell produced by the method of embodiment 169, wherein the plant, plant part, or plant cell is resistant or tolerant to a fungal pathogen.

175. The plant, plant part, or plant cell of embodiment 174, wherein the FIT2 protein is transiently expressed.

176. The plant, plant part, or plant cell of embodiment 174, wherein the FIT2 protein is stably expressed.

177. The plant, plant part, or plant cell of embodiment 174, wherein the plant, plant part, or plant cell is in the subfamily Papilionoideae.

178. The plant, plant part, or plant cell of embodiment 174, wherein the plant, plant part, or plant cell is Glycine spp., Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

179. The plant, plant part, or plant cell of embodiment 174, wherein the plant is Glycine max, wherein the plant, plant part, or plant cell is resistant or tolerant to Asian Soybean Rust caused by Phakopsora pachyrhizi.

180. The plant, plant part, or plant cell of embodiment 174, wherein the plant, plant part, or plant cell is resistant or tolerant to Asian Soybean Rust.

181. The transgenic plant, plant part, or plant cell of embodiment 162, further comprising one or more TIR-NLR signaling transgenes.

182. The transgenic plant, plant part, or plant cell of embodiment 181, wherein the one or more TIR-NLR signaling transgenes is selected from NRG1, SAG101, EDS1, PAD4, and ADR1.

183. A plant, plant part, or plant cell that has been genetically engineered to increase expression of an endogenous FIT2 gene.

184. The plant, plant part, or plant cell of embodiment 183, wherein the plant, plant part, or plant cell is a species of Glycine spp., and wherein the endogenous FIT2 gene encodes a protein at least 90% identical to SEQ ID NO: 16.

185. The plant, plant part, or plant cell of embodiment 184, wherein the increase in expression of the endogenous FIT2 gene is at least 4-fold greater compared to a non-genetically engineered plant, plant part or plant cell.

186. The plant, plant part, or plant cell of embodiment 183, wherein the increase in expression of the endogenous FIT2 gene is at least 4-fold greater compared to a non-genetically engineered plant, plant part, or plant cell.

187. The plant, plant part, or plant cell of embodiment 183, wherein the increase in expression of the endogenous FIT2 gene is at least 16-fold greater compared to a non-genetically engineered plant, plant part, or plant cell.

188. The plant, plant part, or plant cell of embodiment 183, wherein the endogenous FIT2 sequence is inserted into a native, non-essential gene.

189. A method for identifying a functional FIT2 gene and/or allele thereof comprising: isolating a FIT2 homolog or allele thereof; expressing all or a substantial fragment of said FIT2 homolog or allele thereof in combination with a homolog of AvrFIT2 in a plant, plant part, or plant cell; and assaying said plant, plant part, or plant cell for an immune response.

What is claimed is:

1. A recombinant DNA construct comprising at least one of:
a nucleic acid sequence encoding a protein comprising SEQ ID NO: 2, or a protein at least 90% identical thereto;
a nucleic acid sequence encoding a protein comprising SEQ ID NO: 4, or a protein at least 90% identical thereto;
a nucleic acid sequence encoding a protein comprising SEQ ID NO: 6, or a protein at least 90% identical thereto;
a nucleic acid sequence encoding a protein comprising SEQ ID NO: 8, or a protein at least 90% identical thereto;
a nucleic acid sequence encoding a protein comprising SEQ ID NO: 10, or a protein at least 90% identical thereto;
a nucleic acid sequence encoding a protein comprising SEQ ID NO: 12, or a protein at least 90% identical thereto;
a nucleic acid sequence encoding a protein comprising SEQ ID NO: 14, or a protein at least 90% identical thereto;
a nucleic acid sequence encoding a protein comprising SEQ ID NO: 16, or a protein at least 90% identical thereto;
a nucleic acid sequence encoding a protein comprising SEQ ID NO: 18, or a protein at least 90% identical thereto;
a nucleic acid sequence encoding a protein comprising SEQ ID NO: 20, or a protein at least 90% identical thereto;
a nucleic acid sequence encoding a protein comprising SEQ ID NO: 22, or a protein at least 90% identical thereto;
a nucleic acid sequence encoding a protein comprising SEQ ID NO: 24, or a protein at least 90% identical thereto;
a nucleic acid sequence encoding a protein comprising SEQ ID NO: 26, or a protein at least 90% identical thereto;
a nucleic acid sequence encoding a protein comprising SEQ ID NO: 28, or a protein at least 90% identical thereto;
a nucleic acid sequence encoding a protein comprising SEQ ID NO: 30, or a protein at least 90% identical thereto;
a nucleic acid sequence encoding a protein comprising SEQ ID NO: 32, or a protein at least 90% identical thereto; or
combinations thereof,
and wherein the construct is capable of conferring resistance to a fungal pathogen when transformed into a plant.

2. A transgenic plant, plant part, or plant cell having resistance or tolerance to a fungal pathogen, wherein the resistance or tolerance is conferred by a transgene comprising at least one of:
a nucleic acid sequence encoding a protein comprising SEQ ID NO: 2, or a protein at least 90% identical thereto;
a nucleic acid sequence encoding a protein comprising SEQ ID NO: 4, or a protein at least 90% identical thereto;
a nucleic acid sequence encoding a protein comprising SEQ ID NO: 6, or a protein at least 90% identical thereto;
a nucleic acid sequence encoding a protein comprising SEQ ID NO: 8, or a protein at least 90% identical thereto;
a nucleic acid sequence encoding a protein comprising SEQ ID NO: 10, or a protein at least 90% identical thereto;
a nucleic acid sequence encoding a protein comprising SEQ ID NO: 12, or a protein at least 90% identical thereto;
a nucleic acid sequence encoding a protein comprising SEQ ID NO: 14, or a protein at least 90% identical thereto;
a nucleic acid sequence encoding a protein comprising SEQ ID NO: 16, or a protein at least 90% identical thereto;
a nucleic acid sequence encoding a protein comprising SEQ ID NO: 18, or a protein at least 90% identical thereto;
a nucleic acid sequence encoding a protein comprising SEQ ID NO: 20, or a protein at least 90% identical thereto;
a nucleic acid sequence encoding a protein comprising SEQ ID NO: 22, or a protein at least 90% identical thereto;
a nucleic acid sequence encoding a protein comprising SEQ ID NO: 24, or a protein at least 90% identical thereto;
a nucleic acid sequence encoding a protein comprising SEQ ID NO: 26, or a protein at least 90% identical thereto;
a nucleic acid sequence encoding a protein comprising SEQ ID NO: 28, or a protein at least 90% identical thereto;
a nucleic acid sequence encoding a protein comprising SEQ ID NO: 30, or a protein at least 90% identical thereto;
a nucleic acid sequence encoding a protein comprising SEQ ID NO: 32, or a protein at least 90% identical thereto; or
combinations thereof.

3. The transgenic plant, plant part, or plant cell of claim 2, wherein the fungal pathogen is from the order Pucciniales or Cantharellales.

4. The transgenic plant, plant part, or plant cell of claim 3, wherein the fungal pathogen is Phakopsora pachyrhizi, Rhizoctonia solani, Melampsora spp., Phakopsora meibomiae, Phakopsora euvitis, Phakopsora spp., Puccinia spp., Uromyces spp., Austropuccinia spp., Cronartium spp., Austropuccinia spp., Cronartium spp. or Hemileia vastatrix.

5. The transgenic plant, plant part, or plant cell of claim 2, wherein the plant, plant part, or plant cell is in the subfamily Papilionoideae.

6. The transgenic plant, plant part, or plant cell of claim 2, wherein the plant, plant part, or plant cell is Glycine spp., Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

7. The transgenic plant, plant part, or plant cell of claim 6, wherein the plant, plant part, or plant cell is Glycine max, and wherein the plant, plant part, or plant cell is resistant to Asian Soybean Rust caused by Phakopsora pachyrhizi.

8. The transgenic plant, plant part, or plant cell of claim 7, wherein the resistance to Asian Soybean Rust is conferred by a transgene comprising SEQ ID NO: 1, or by a sequence at least 70% identical thereto.

9. A method of producing a plant, plant part, or plant cell having resistance or tolerance to a fungal pathogen, wherein the method comprises:
transforming a plant, plant part, or plant cell with an isolated, recombinant, or synthetic polynucleotide comprising:
a nucleic acid sequence encoding a functional FIT2 protein, wherein the
nucleic acid sequence is at least 70% identical to SEQ ID NO: 1 and
selecting a plant comprising the polynucleotide and having resistance or tolerance to a fungal pathogen.

10. The method of claim 9, wherein the nucleotide sequence encoding the FIT2 protein has been codon optimized.

11. The method of claim 9, wherein the plant, plant part, or plant cell is transformed with two or more polynucleotides encoding different FIT2 proteins.

12. The method of claim 9, wherein the plant, plant part, or plant cell is transformed or introgressed with one or more additional desired traits.

13. The method of claim 9, wherein the one or more additional desired traits are resistance traits to a disease, pest, or abiotic stress.

14. A plant, plant part, or plant cell produced by the method of claim 9, wherein the plant, plant part, or plant cell is resistant or tolerant to a fungal pathogen.

15. The plant, plant part, or plant cell of claim 14, wherein the FIT2 protein is transiently expressed.

16. The plant, plant part, or plant cell of claim 14, wherein the FIT2 protein is stably expressed.

17. The plant, plant part, or plant cell of claim 14, wherein the plant, plant part, or plant cell is in the subfamily Papilionoideae.

18. The plant, plant part, or plant cell of claim 14, wherein the plant, plant part, or plant cell is Glycine spp., Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

19. The plant, plant part, or plant cell of claim 14, wherein the plant is Glycine max, wherein the plant, plant part, or plant cell is resistant or tolerant to Asian Soybean Rust caused by Phakopsora pachyrhizi.

20. The plant, plant part, or plant cell of claim 14, wherein the plant, plant part, or plant cell is resistant or tolerant to Asian Soybean Rust.

21. The transgenic plant, plant part, or plant cell of claim 2, further comprising one or more TIR-NLR signaling transgenes.

22. The transgenic plant, plant part, or plant cell of claim 21, wherein the one or more TIR-NLR signaling transgenes is selected from NRG1, SAG101, EDS1, PAD4, and ADR1.

23. A plant, plant part, or plant cell that has been genetically engineered to increase expression of an endogenous FIT2 gene.

24. The plant, plant part, or plant cell of claim 23, wherein the plant, plant part, or plant cell is a species of Glycine spp., and wherein the endogenous FIT2 gene encodes a protein at least 90% identical to SEQ ID NO: 16.

25. The plant, plant part, or plant cell of claim 24, wherein the increase in expression of the endogenous FIT2 gene is at least 4-fold greater compared to a non-genetically engineered plant, plant part or plant cell.

26. The plant, plant part, or plant cell of claim 23, wherein the increase in expression of the endogenous FIT2 gene is at least 4-fold greater compared to a non-genetically engineered plant, plant part, or plant cell.

27. The plant, plant part, or plant cell of claim 23, wherein the increase in expression of the endogenous FIT2 gene is at least 16-fold greater compared to a non-genetically engineered plant, plant part, or plant cell.

28. The plant, plant part, or plant cell of claim 23, wherein the endogenous FIT2 sequence is inserted into a native, non-essential gene.

29. A method for identifying a functional FIT2 gene and/or allele thereof comprising:
isolating a FIT2 homolog or allele thereof,
expressing all or a substantial fragment of said FIT2 homolog or allele thereof in combination with a homolog of AvrFIT2 in a plant, plant part, or plant cell; and
assaying said plant, plant part, or plant cell for an immune response.
